(19) United States
(12) Patent Application Publication
(10) Pub. No.: US 2026/0306810 A1
(43) Pub. Date: Oct. 8, 2026

(54) INGESTIBLE DEVICES, ASSEMBLIES, AND METHODS FOR DELIVERING A THERAPEUTIC AGENT

(71) Applicant: Rani Therapeutics, LLC, San Jose, CA (US)

(72) Inventor: Mir IMRAN, Los Altos Hills, CA (US)

(73) Assignee: Rani Therapeutics, LLC, San Jose, CA (US)

(21) Appl. No.: 19/542,489

(22) Filed: Feb. 17, 2026

Related U.S. Application Data
(63) Continuation of application No. PCT/US2024/042610, filed on Aug. 16, 2024.
(60) Provisional application No. 63/520,558, filed on Aug. 18, 2023.
(60) Provisional application No. 63/591,503, filed on Oct. 19, 2023.
(60) Provisional application No. 63/609,739, filed on Dec. 13, 2023.
(60) Provisional application No. 63/625,112, filed on Jan. 25, 2024.

(51) Int. Cl. A61M 31/00; A61M 37/00; A61M 39/00; A61M 39/24

(52) U.S. Cl. CPC A61M 31/002; A61M 37/00; A61M 39/24; A61M 2039/0009; A61M 2039/242; A61M 2202/0007; A61M 2202/049; A61M 2205/0216; A61M 2205/3331; A61M 2210/1017; A61M 2210/1042

(57) ABSTRACT

A payload delivery module for an ingestible device includes a piston, a hollow needle, a membrane, a fluid dosage form, and a valve. The piston has an interior. The needle is coupled to the piston. The membrane is coupled to the piston to define a reservoir in the interior. The fluid dosage form is disposed in the reservoir and includes at least one therapeutic agent. The valve is coupled to the piston to control a flow of the fluid dosage form from the reservoir to the needle.

### CROSS-REFERENCE TO RELATED APPLICATIONS

This application is a continuation of International Patent Application no. PCT/US2024/042610 filed on Aug. 16, 2024, which claims the benefit of and priority to U.S. Provisional Application No. 63/520,558, filed on Aug. 18, 2023; 63/591,503, filed on Oct. 19, 2023; 63/609,739, filed on Dec. 13, 2023; and 63/625,112, filed on Jan. 25, 2024. The entire contents of the foregoing applications are hereby incorporated by reference herein.

### BACKGROUND

A therapeutic agent such as a drug may be administered to a subject by ingestion or through parenteral injection (e.g., subcutaneously, intramuscularly, or intravenously) to provide a desired therapeutic effect. However, these routes of administration have some disadvantages. For example, some therapeutic agents such as biologics including large (macro) molecules are not suitable for delivery by ingestion because of enzymatic breakdown of these molecules in the gastrointestinal (GI) tract of a subject and/or inability to pass through the tight junctions of the epithelial layer of the GI tract wall. Other types of therapeutic agents may otherwise be poorly tolerated within the GI tract resulting in low systemic uptake. With parenteral injections, subjects may experience pain and inconvenience with administration which can significantly impact compliance and quality of life.

Ingestible devices have been proposed to address some of the challenges associated with traditional oral and parenteral delivery of various therapeutic agents. For example, some ingestible devices are designed to actively deliver a therapeutic agent into the GI wall tissue. However, most of these devices have limited design flexibility. For example, most devices are unable to incorporate different delivery mechanisms with different delivery modes to, for example, deliver different dosage forms (e.g., solid and liquid dosage forms). Thus, to be able to incorporate different delivery mechanisms on the same device platform, most of these devices would have to undergo a substantial redesign, require a significant number of additional components, or require a different device platform to be used altogether. Accordingly, it may be desirable to provide an ingestible device with sufficient design flexibility to, for example, be able to incorporate different delivery mechanisms.

These and other challenges encountered with existing drug delivery modalities are addressed by embodiments of the present disclosure. The provided ingestible devices, payload delivery modules, delivery assemblies, methods of making them, and methods and uses thereof for delivering one or more therapeutic agents also may offer one or more additional advantages over existing drug delivery devices.

### SUMMARY

Embodiments of the present disclosure relate generally to ingestible devices, assemblies, and methods for delivering one or more therapeutic agents into a GI lumen wall or surrounding tissue thereof (e.g., the peritoneum or peritoneal cavity) of a subject. In some embodiments, the devices and assemblies disclosed herein can have a modular design to be able to incorporate different delivery mechanisms with different delivery modes to enable the delivery of different dosage forms and/or dosage amounts using interchangeable components on the same device platform. In this manner, the devices and assemblies disclosed herein can allow for the use of a common device platform to deliver a broad range of dosages and dosage forms to accommodate most therapeutic regimens. These and other advantageous features will become apparent to those of skill in the art reading this disclosure.

In one aspect, a payload delivery module for an ingestible device includes a piston, a hollow needle, a membrane, a fluid dosage form, and a valve. The piston includes an interior. The hollow needle is coupled to the piston. The membrane is coupled to the piston to define a reservoir in the interior. The fluid dosage form is disposed in the reservoir and includes at least one therapeutic agent. The valve is coupled to the piston to control a flow of the fluid dosage form from the reservoir to the needle.

In another aspect, a payload delivery module for an ingestible device includes a piston, a hollow needle, a plunger, a fluid dosage form, and a valve. The piston includes an interior. The hollow needle is coupled to the piston. The plunger is movably coupled to the piston to define a reservoir in the interior. The fluid dosage form is disposed in the reservoir and includes at least one therapeutic agent. The valve is coupled to the piston to control a flow of the fluid dosage form from the reservoir to the needle.

In another aspect, a payload delivery module for an ingestible device includes a piston, a cartridge, and a solid dosage form. The piston includes a base and an elongated section. The cartridge includes a container. The solid dosage form is disposed in the container and includes at least one therapeutic agent.

In another aspect, a payload delivery module for an ingestible device includes a piston, a membrane, and a valve. The piston includes an interior, a needle port, and a fill port. The membrane is coupled to the piston to define a reservoir in the interior for containing a fluid dosage form. The valve is coupled to the piston between the membrane and the needle port.

In another aspect, a payload delivery module for an ingestible device includes a piston, a plunger, and a valve. The piston includes an interior, a needle port, and a fill port. The plunger is movably coupled to the piston to define a reservoir in the interior for containing a fluid dosage form. The valve is coupled to the piston between the plunger and the needle port.

In one or more embodiments of any of the foregoing aspects, the piston is structured to be releasably coupled to, and movably disposed in, a housing.

In one or more embodiments of any of the foregoing aspects, the valve is structured to control a flow of the fluid dosage form from the reservoir to the needle in response to axial movement of the module relative to a housing.

In one or more embodiments of any of the foregoing aspects, the piston includes a lateral wall defining a release feature for releasably coupling the piston to a housing. In some embodiments, the release feature is defined by an outer circumferential surface of the piston. In some embodiments, the release feature includes a press-fit feature.

In one or more embodiments of any of the foregoing aspects, the valve includes a seal coupled to the piston to contain the fluid dosage form in the reservoir. In some embodiments, the valve further includes a puncture member coupled to the piston adjacent to the seal, and the puncture member is structured to move relative to the piston to pierce the seal. In some embodiments, the puncture member is structured to create a substantially fluid-tight seal with the piston upon piercing the seal. In some embodiments, the valve further includes a base projecting from an outer surface of the piston and a tip coupled to the base. In some embodiments, the base is integral with the piston. In some embodiments, the base is structured to deform to cause the tip to move relative to the piston to pierce the seal.

In one or more embodiments of any of the foregoing aspects, the valve includes a base projecting from an outer surface of the piston and a plug releasably coupled to the piston adjacent to the base. In some embodiments, the plug creates a substantially fluid tight seal with the piston between the membrane and the needle for containing the fluid dosage form in the reservoir. In some embodiments, the base is structured to deform to cause the plug to release from the piston and allow the fluid dosage form to flow to the needle.

In one or more embodiments of any of the foregoing aspects, the membrane includes a flexible material to allow for deformation of the membrane upon the valve opening by a pressure applied against an outer surface of the membrane.

In one or more embodiments of any of the foregoing aspects, the membrane includes a metalized film or coating.

In one or more embodiments of any of the foregoing aspects, the plunger is structured to move relative to the piston upon the valve opening in response to a pressure applied against an outer surface of the plunger.

In one or more embodiments of any of the foregoing aspects, the reservoir defines a volume for containing up to about 250 μl of fluid. In some embodiments, the reservoir defines a volume for containing about 50-200 μl of fluid.

In one or more embodiments of any of the foregoing aspects, the needle has a critical length in a range of about 3.5-12 mm such that the needle delivers the fluid dosage form through the GI lumen wall into the peritoneal cavity.

In one or more embodiments of any of the foregoing aspects, the needle has a critical length in a range of about 1.5-3 mm such that the needle delivers the fluid dosage form into a layer of the GI lumen wall.

In one or more embodiments of any of the foregoing aspects, the needle includes a biodegradable material to allow for degradation of at least a portion of the needle in a GI lumen wall or surrounding tissue thereof. In some embodiments, the needle includes a body and a tip coupled to the body, wherein the body is formed from a first material and the tip is formed from a second material, and wherein the second material has a hardness that is greater than a hardness of the first material.

In one or more embodiments of any of the foregoing aspects, the payload delivery module further includes a cover releasably coupled to the piston. In some embodiments, the cover defines part of a housing for holding the payload delivery module.

In one or more embodiments of any of the foregoing aspects, the solid dosage form is shaped as or is contained in a needle structure. In some embodiments, the elongated section of the piston is structured to eject the needle structure from the container as a projectile into the GI lumen wall or surrounding tissue thereof.

In one or more embodiments of any of the foregoing aspects, the at least one therapeutic agent is one or more selected from a small molecule, a peptide, a polypeptide, a protein, a hormone, an antibody, or a nucleic acid.

In one or more embodiments of any of the foregoing aspects, the at least one therapeutic agent is one or more selected from an immunosuppressive drug, a chemotherapy drug, a central nervous system (CNS) drug, an antidiabetic drug, an enzyme replacement therapy (ERT) drug, an anti-infective drug, a C-type natriuretic peptide (CNP), a programmed death-ligand 1 (PD-L1) protein, a monoclonal antibody, an anti-coagulant, a blood clotting factor, insulin, an incretin or a combination thereof, or an oligonucleotide.

In one or more embodiments of any of the foregoing aspects, the at least one therapeutic agent includes an anti-sense oligonucleotide (ASO). In some embodiments, the ASO is MALAT1. In one or more embodiments of any of the foregoing aspects, the at least one therapeutic agent includes one or more blood clotting factors or mimetics thereof selected from Factor VIII, Factor IX, or Factor X. In one or more embodiments of any of the foregoing aspects, the at least one therapeutic agent includes an anti-PCSK9 antibody. In one or more embodiments of any of the foregoing aspects, the at least one therapeutic agent includes a TNF-α inhibiting antibody. In some embodiments, the TNF-α inhibiting antibody includes adalimumab or an analogue thereof. In one or more embodiments of any of the foregoing aspects, the at least one therapeutic agent includes an anti-interleukin antibody. In some embodiments, the anti-interleukin antibody targets interleukin 4 and interleukin 13. In some embodiments, the anti-interleukin antibody includes dupilumab or an analogue thereof. In some embodiments, the anti-interleukin antibody targets at least one of interleukin 12 or interleukin 23. In some embodiments, the anti-interleukin antibody includes ustekinumab or an analogue thereof. In some embodiments, the anti-interleukin antibody targets interleukin 2. In one or more embodiments of any of the foregoing aspects, the at least one therapeutic agent includes parathyroid hormone (PTH) or an analogue thereof. In one or more embodiments of any of the foregoing aspects, the at least one therapeutic agent includes amylin or an analogue thereof. In one or more embodiments of any of the foregoing aspects, the at least one therapeutic agent includes one or more incretins or mimetics thereof selected from GLP-1, GLP-2, GIP, PYY, or glucagon receptor agonists.

In another aspect, provided herein are delivery assemblies for an ingestible device including a housing and a payload delivery module as described herein coupled to the housing. In some embodiments, the housing includes a proximal end, a distal end, a piston chamber located between the proximal and distal ends, and a housing release feature. The proximal end includes a first opening for receiving a force in the interior and the distal end includes a second opening for discharging the dosage form. In some embodiments, the housing further includes a body and a cover coupled to the body. The body includes the second opening and the piston chamber, and the cover includes the first opening and the housing release feature. In some embodiments, the piston is disposed in the piston chamber and is releasably coupled to the housing release feature. In some embodiments, a seal is coupled to the housing at the second opening. In some embodiments, the housing further includes a vent channel for venting a gas. In some embodiments, the force is a gas pressure. In some embodiments, the housing release feature is structured to release the piston in response to a threshold gas pressure applied to the module through the first opening, and the threshold gas pressure is about 10-40 psi. In some embodiments, the housing release feature is located on a lateral wall of the housing. In some embodiments, the housing release feature includes a press-fit feature. In some embodiments, the housing release feature extends circumferentially about the housing. In some embodiments, when the payload delivery module includes the fluid dosage form, the module is structured to move axially in the piston chamber to insert the hollow needle through the second opening into the GI lumen wall or surrounding tissue and to expel the fluid dosage form through the hollow needle into the GI lumen wall or surrounding tissue in response to the force received through the first opening. In some embodiments, the fluid dosage form is expelled from the reservoir upon the valve opening in response to sufficient axial movement of the module relative to the housing. In some embodiments, the fluid dosage form is expelled through the hollow needle after the hollow needle penetrates the GI lumen wall or surrounding tissue thereof. In some embodiments, when the payload delivery module includes the solid dosage form, the cartridge is coupled to the housing and the piston is structured to move axially in the piston chamber in response to the force received through the first opening to eject the solid dosage form from the container as a projectile into the GI lumen wall or surrounding tissue thereof.

In another aspect, provided herein are ingestible devices including an expandable member and a delivery assembly as described herein coupled to the expandable member. In some embodiments, the device further includes a gas generating mechanism coupled to the expandable member. In some embodiments, the gas generating mechanism is structured to generate a gas to cause the expandable member to expand at a location in a GI tract of a subject to orient and position the delivery assembly relative to a GI lumen wall. In some embodiments, the location is a small intestine of the subject. In some embodiments, the gas generating mechanism includes a plurality of reactants separated from each other by a degradable release. In some embodiments, upon expansion of the expandable member, the delivery assembly is structured to deliver the dosage form through the GI lumen wall and into a peritoneum or a peritoneal cavity of the subject for systemic uptake of the at least one therapeutic agent. In some embodiments, upon expansion of the expandable member, the delivery assembly is structured to deliver the dosage form into a layer of the GI lumen wall for systemic uptake of the at least one therapeutic agent. In some embodiments, the expandable member includes a balloon. In some embodiments, the device further includes an ingestible enclosure and the expandable member and the delivery assembly are disposed in the ingestible enclosure. In some embodiments, the ingestible enclosure includes a biodegradable material to allow for degradation of at least a portion of the ingestible enclosure within the GI tract. In some embodiments, the device further includes a coating disposed over at least a portion of the ingestible enclosure. The coating is structured to degrade at a selected pH in the GI tract. In some embodiments, the ingestible enclosure is a size 00 or size 000 swallowable capsule.

In another aspect, provided herein are methods of preparing an ingestible device for delivering a therapeutic agent into a GI lumen wall or surrounding tissue of a subject including filling a dosage form including a therapeutic agent into a payload delivery module as described herein.

In another aspect, provided herein are methods of delivering a therapeutic agent into a GI lumen wall or surrounding tissue of a subject in need thereof including ingesting, by the subject, an ingestible device as described herein.

In another aspect, provided herein are methods of delivering one or more therapeutic agents to a patient in need thereof by swallowing an ingestible device as described herein. Likewise, in some embodiments, provided herein are uses of an ingestible device as described herein for delivering one or more therapeutic agents to a patient in need thereof by swallowing of the ingestible device. In some embodiments, the at least one therapeutic agent includes a therapeutically effective dose of the therapeutic agent in liquid form. Upon swallowing the device, the expandable member expands in a GI tract of the patient to thereby deliver the therapeutically effective dose of the therapeutic agent into a lumen wall or surrounding tissue of the GI tract. In some embodiments, the at least one therapeutic agent includes a therapeutically effective dose of an oligonucleotide. In some embodiments, the oligonucleotide includes an anti-sense oligonucleotide (ASO). In some embodiments, the ASO includes MALAT1 ASO. In some embodiments, the at least one therapeutic agent includes a therapeutically effective dose of a TNF-α inhibiting antibody. In some embodiments, the TNF-α inhibiting antibody includes adalimumab. In some embodiments, the adalimumab is HUMIRA® or a biosimilar thereof. In some embodiments, the therapeutically effective dose of adalimumab is about 3-11 mg. In some embodiments, the at least one therapeutic agent includes a therapeutically effective dose of an anti-interleukin antibody. In some embodiments, the anti-interleukin antibody targets interleukin 4 and interleukin 13. In some embodiments, the anti-interleukin antibody includes dupilumab. In some embodiments, the dupilumab is DUPIXENT® or a biosimilar thereof. In some embodiments, the therapeutically effective dose of dupilumab is about 16-30 mg. In some embodiments, the therapeutically effective dose of dupilumab is about 20-25 mg. In some embodiments, the anti-interleukin antibody targets at least one of interleukin 12 or interleukin 23. In some embodiments, the anti-interleukin antibody includes ustekinumab. In some embodiments, the ustekinumab is STELARA® or a biosimilar thereof. In some embodiments, the therapeutically effective dose of the anti-interleukin antibody delivered to the patient produces a bioavailability in the patient that is substantially the same as a bioavailability of a subcutaneously administered dose of the anti-interleukin antibody. In some embodiments, the therapeutically effective dose of the anti-interleukin antibody delivered to the patient produces a bioavailability in the patient that is higher than a bioavailability of a subcutaneously administered dose of the anti-interleukin antibody. In some embodiments, the therapeutically effective dose of the anti-interleukin antibody delivered to the patient produces a Cₘₐₓ in the patient that is greater than a Cₘₐₓ of a subcutaneously administered dose of the anti-interleukin antibody. In some embodiments, the therapeutically effective dose of the anti-interleukin antibody delivered to the patient produces a tₘₐₓ in the patient that is less than a tₘₐₓ of a subcutaneously administered dose of the anti-interleukin antibody. In some embodiments, the at least one therapeutic agent includes a therapeutically effective dose of at least one incretin. In some embodiments, the at least one incretin is an incretin triagonist including GLP-1, GIP, and glucagon receptor agonists. In some embodiments, the therapeutically effective dose of the incretin triagonist delivered to the patient produces a weight loss in the patient that is substantially the same as a weight loss produced by a subcutaneously administered dose of the incretin triagonist.

In another aspect, a modular delivery assembly for an ingestible device for delivering a dosage form into a GI lumen wall or surrounding tissue thereof of a subject includes a housing having a proximal end, a distal end, and an interior located between the proximal and distal ends. The proximal end includes a first opening for receiving a force in the interior. The distal end includes a second opening for discharging the dosage form. The interior includes a receptacle structured to receive a module selected from a first payload delivery module including a fluid dosage form and a second payload delivery module including a solid dosage form. The housing further includes a release feature to releasably couple the module to the housing. In some embodiments, the first payload delivery module has a first delivery mode and the second payload delivery module has a second delivery mode different from the first delivery mode. In some embodiments, the first delivery mode includes inserting a hollow needle into the GI lumen wall or surrounding tissue and discharging the fluid dosage form through the hollow needle into the GI lumen wall or surrounding tissue. In some embodiments, the second delivery mode includes ejecting the solid dosage form from the second payload delivery module as a projectile into the GI lumen wall or surrounding tissue.

In another aspect, a delivery assembly for an ingestible device includes a housing and a payload delivery module. The housing includes a proximal end, a distal end, a piston chamber located between the proximal and distal ends, and a release feature. The proximal end includes a first opening for receiving a force in the interior and the distal end includes a second opening for discharging the dosage form. The payload delivery module is disposed in the housing and includes a piston and a dosage form including at least one therapeutic agent. The piston is releasably coupled to the release feature and is structured to move axially in the piston chamber between the proximal and distal ends in response to the force to discharge the dosage form from the housing.

In another aspect, an ingestible device includes an expandable member and a delivery assembly as described herein coupled to the expandable member. The delivery assembly includes a fluid dosage form including at least one therapeutic agent. The expandable member is structured to expand in a GI tract of a subject to position the delivery assembly relative to a GI lumen wall. Upon expansion of the expandable member, the delivery assembly is structured to deliver the fluid dosage form through the GI lumen wall and into a peritoneum or a peritoneal cavity of the subject for systemic uptake of the at least one therapeutic agent.

In another aspect, an ingestible device includes a housing, a piston, a hollow needle, a fluid dosage form, and a force generating mechanism. The piston is movably disposed in the housing. The hollow needle is coupled to the piston. The fluid dosage form is disposed in the housing and includes at least one therapeutic agent. The force generating mechanism is operatively coupled to the piston. In some embodiments, the hollow needle has a critical length in a range of about 3.5-12 mm such that upon ingestion of the device, the hollow needle is structured to deliver the fluid dosage form through a GI lumen wall into a peritoneal cavity of a subject for systemic uptake of the at least one therapeutic agent. In some embodiments, the hollow needle has a critical length in a range of about 1.5-3 mm such that upon ingestion of the device, the hollow needle is structured to deliver the fluid dosage form into a layer of the GI lumen wall for systemic uptake of the at least one therapeutic agent.

The foregoing general description and following detailed description are provided by way of example and are intended to provide further explanation without being limiting. Other objects, advantages, and novel features will be readily apparent to those skilled in the art from the following brief description of the drawings and detailed description.

### BRIEF DESCRIPTION OF THE DRAWINGS

FIG. 1 is a cross-sectional view of an example of an ingestible device including a modular delivery assembly that can incorporate different delivery mechanisms.

FIG. 2 is a partial cutaway view illustrating an example of the ingestible device of FIG. 1 in a folded state disposed in a swallowable capsule.

FIG. 3 is a cross-sectional view of an example modular housing for the ingestible device of FIG. 1.

FIG. 4 is an exploded view of two different modular arrangements of a modular delivery assembly for the ingestible device of FIG. 1.

FIG. 5 is a partial cross-sectional view of an example of the ingestible device of FIG. 1 including a first payload delivery module having a fluid dosage form.

FIG. 6 is a partial cross-sectional view of the device of FIG. 5 in a second state when the fluid dosage form is discharged from the device.

FIG. 7 is a partial cross-sectional view of another example of the ingestible device of FIG. 1 including another version of first payload delivery module with a different valve arrangement.

FIG. 8 is a partial cross-sectional view of the device of FIG. 7 in a second state when the fluid dosage form is discharged from the device.

FIG. 9 is a partial cross-sectional view of another example of the ingestible device of FIG. 1 including yet another version of the first payload delivery module with a different valve arrangement.

FIG. 10 is a partial cross-sectional view of the device of FIG. 9 in a second state when the fluid dosage form is discharged from the device.

FIG. 11 is a partial cross-sectional view of another example of the ingestible device of FIG. 1 including yet another version of the first payload delivery module.

FIG. 12 is a perspective view of a delivery assembly of the ingestible device of FIG. 11.

FIG. 13 is a partial cross-sectional view of a payload delivery module of the device of FIG. 11 prior to filling with a fluid dosage form.

FIG. 14 is a partial cross-sectional view of the payload delivery module of FIG. 13 after filling with a fluid dosage form.

FIG. 15 is an exploded view of the payload delivery module of FIG. 13.

FIGS. 16-19 illustrate an actuation sequence of the device of FIG. 11 in a GI tract of a subject for delivering a fluid dosage form into the GI lumen wall or surrounding tissue thereof.

FIG. 20 is a partial cross-sectional view of another example of the ingestible device of FIG. 1 including yet another version of the first payload delivery module.

FIGS. 21-22 illustrate an example of a needle for delivering a fluid dosage form.

FIGS. 23-24 illustrate another example of a needle for delivering a fluid dosage form.

FIGS. 25-27 illustrate an example method of filling a payload delivery module with a fluid dosage form.

FIG. 28 is a partial cross-sectional view of another example of the ingestible device of FIG. 1 including a second payload delivery module having a solid dosage form.

FIG. 29 is a partial cross-sectional view of the device of FIG. 28 in a second state when the solid dosage form is ejected from the device.

FIG. 30 is a partial cross-sectional view of an example delivery assembly having a different housing for receiving the second payload delivery module of FIG. 28.

FIG. 31 is a block diagram illustrating an example method of delivering a dosage form from an ingestible device as described herein.

FIG. 32 shows the plasma concentration vs. time curve obtained after administration of a single dose of metastasis associated lung adenocarcinoma transcript 1 anti-sense oligonucleotide (MALAT1 ASO) in a canine study (0.5 mg/kg of MALAT1 ASO by intrajejunal injection to mimic oral delivery via an ingestible device as described herein, or 0.5 mg/kg of MALAT1 ASO by subcutaneous injection).

FIG. 33 shows the plasma concentration vs. time curve obtained after administration of a single dose of adalimumab in a canine study (11 mg dose of adalimumab in liquid form administered orally to four awake dogs via an ingestible device as described herein, or 4.5 mg dose of adalimumab in solid form administered orally to two awake dogs via an ingestible device configured to deliver its payload into a GI lumen wall or surrounding tissue, or 5 mg dose of adalimumab in liquid form administered via subcutaneous injection to three dogs).

FIG. 34 shows the plasma concentration vs. time curve obtained after administration of a single dose of dupilumab in a canine study (16.5 mg or 30 mg dose of dupilumab in liquid form administered orally via an ingestible device as described herein to six awake dogs, respectively, or 16.5 mg or 30 mg dose of dupilumab in liquid form administered via subcutaneous injection to three dogs, respectively).

FIG. 35 shows time course changes in body weight obtained after administration of a single dose of an incretin triagonist in a canine study (0.12 mg/kg dose of RTJH23 incretin triagonist administered by intrajejunal injection to mimic oral delivery via an ingestible device as described herein, or 0.12 mg/kg by subcutaneous injection).

FIG. 36 shows peak decreases in body weights and serum lipids from the same canine study of FIG. 35.

FIG. 37 shows the plasma concentration vs. time curve obtained after administration of a single dose of ustekinumab in a canine study (18 mg dose of ustekinumab in liquid form administered orally via an ingestible device as described herein to four awake dogs, or 18 mg dose of ustekinumab in liquid form administered via subcutaneous injection to three dogs).

### DETAILED DESCRIPTION

Before discussing details of the devices, assemblies, and methods of the present disclosure, a few conventions are provided for the convenience of the reader.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural references unless the context clearly dictates otherwise. Reference to an object in the singular is not intended to mean “one and only one” unless explicitly so stated, but rather “one or more.”

As used herein, a phrase in the form “A/B” or in the form “A and/or B” means (A), (B), or (A and B); a phrase in the form “at least one of A, B, or C” means (A), (B), (C), (A and B), (A and C), (B and C), or (A, B, and C).

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, Ph, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “body” refers herein to an animalia body, unless the context clearly dictates otherwise.

The term “subject” refers herein to a body into which an embodiment of the present disclosure is, or is intended to be, delivered. For example, with respect to humans, a subject may be a patient under treatment of a health care professional. The terms “individual,” “subject,” and “patient” may be used interchangeably herein, and refer to any individual animalia subject (e.g., bovine, canine, feline, equine, or human). In specific embodiments, the subject, individual, or patient is a human.

The term “fluid” refers herein to a liquid or gas and encompasses moisture and humidity unless the context dictates otherwise. The term “fluidic environment” refers herein to an environment in which one or more fluids are present.

The term “ingest” or a grammatical variation thereof (e.g., “ingesting”, “ingestion,” or “ingested”) refers herein to taking into the stomach, whether by swallowing or by other means of depositing into the stomach (e.g., by depositing into the stomach by endoscope or depositing into the stomach via a port).

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking. The term “non-degradable” refers to an expectation that degradation will be minimal, or within a certain acceptable design percentage, for at least an expected duration in an expected environment.

The term “degradation rate” or a grammatical variation thereof (e.g., “rate of degradation”) refers herein to a rate at which a material degrades. A designed degradation rate of a material in a particular implementation can be defined by a rate at which the material is expected to degrade under expected conditions (e.g., in physiological conditions) at a target delivery site. A designed degradation time for a particular implementation can refer to a designed time to complete degradation or a designed time to a partial degradation sufficient to accomplish a design purpose (e.g., breach). Accordingly, for example, a designed degradation time can be specific to a component and/or specific to expected conditions at a target delivery site. A designed degradation time can be short or long and can be defined in terms of approximate times, maximum times, or minimum times.

The terms “substantially”, “about”, and similar terms are used herein to describe and account for small variations which may result from, for example, a manufacturing or assembly process. For example, when used in conjunction with a numerical value, the terms refer to a variation in the value of less than or equal to ±10%.

The term “lumen” refers herein to the inside space of a tubular structure. Examples of lumens in a body include arteries, veins, and tubular cavities within organs.

The term “lumen wall” refers to a wall of a lumen, where the wall includes all layers from an inner perimeter to an outer perimeter of the lumen, such as, with respect to lumens in a body, the mucosa, submucosa, muscularis, serosa, and an outer wall of the lumen, with the constituent blood vessels and tissues.

The term “gastrointestinal tract” or “GI tract” refers herein to the intake/expulsion system of a body including, for example, the mouth, pharynx, esophagus, stomach, pylorus, small intestine, cecum, large intestine, colon, rectum, anus, and valves or sphincters therebetween.

The term “GI lumen” refers generally to any lumen of the GI tract (e.g., a lumen of the esophagus, stomach, small intestine, large intestine, or colon) and the term “GI lumen wall” refers to a lumen wall of a GI lumen.

As used herein, the terms “comprising”, “comprise”, “comprises”, “includes”, and “including” are intended to mean that the compositions and methods include the recited elements, but do not exclude others.

Referring generally to the Figures, disclosed herein are embodiments relating to ingestible devices, assemblies, and methods for autonomously delivering a dosage form including one or more therapeutic agents into a GI lumen wall or surrounding tissue thereof (e.g., such as the peritoneum or peritoneal cavity) of a subject. In some embodiments, the devices and assemblies disclosed herein may have a modular design to be able to incorporate different delivery mechanisms with different delivery modes to enable the delivery of different dosage forms and dosage amounts using interchangeable components on the same device platform. In this manner, the devices and assemblies disclosed herein can allow for the use of a common device platform to orally deliver a broad range of dosages of various therapeutic agents to accommodate most therapeutic regimens.

Also disclosed herein are ingestible devices, payload delivery modules, delivery assemblies, methods of making them, and methods and uses thereof for delivering one or more therapeutic agents that are advantageous in one or more respects over existing ingestible drug delivery devices.

According to a non-limiting example of the present disclosure, an ingestible device includes an expandable member and a delivery assembly coupled to the expandable member. The delivery assembly can include a modular housing that is advantageously structured to incorporate different payload delivery modules for the oral delivery of different dosage forms and/or dosage amounts.

For example, in a first configuration, the delivery assembly can include a first payload delivery module including a piston containing a fluid dosage form and a hollow needle coupled to the piston. The piston can be disposed in the housing and releasably coupled to a release feature or trigger defined by the housing and piston. After ingestion of the ingestible device, the expandable member can expand at a desired location in a GI tract of a subject for delivery of the fluid dosage form. Upon expansion of the expandable member within the GI lumen, the piston can release from the release feature to move within the housing and insert the hollow needle into the GI lumen wall or surrounding tissue. As described herein, expansion of the expandable member can also provide the required force to expel the fluid dosage form through the hollow needle and into the GI lumen wall or surrounding tissue thereof for systemic uptake of the therapeutic agent(s) contained in the fluid dosage form.

In a second configuration different from the first configuration, the delivery assembly can alternatively include a second payload delivery module including a piston and a cartridge containing a solid dosage form. The piston may have a different structure from the piston used in the first payload delivery module. However, the piston in this configuration can be disposed in the same housing discussed above and releasably coupled to the housing via the same release feature or trigger arrangement. The cartridge can also be coupled to, and disposed in, the same housing.

In other examples, the housing may have a different structure to accommodate the cartridge for delivery of the solid dosage form but can include the same release feature or trigger arrangement as the first configuration discussed above to enable use with the same expandable member.

In other examples, the housing may have a different trigger arrangement but can be coupled to the same expandable member and have the same actuation sequence as the first configuration.

In still other examples, the second payload delivery module can be substantially the same as the first payload delivery module, but the piston can be structured to contain a different volume of the fluid dosage form to provide dosing flexibility.

In any of the foregoing configurations, the devices can be structured to deliver the dosage form using substantially the same actuation mechanics and actuation sequence.

For example, after ingestion of the ingestible device, the expandable member can expand at a desired location in a GI tract of the subject for delivery of the solid dosage form. Upon expansion of the expandable member in the GI lumen, the piston can release from the release feature in the same manner as the first payload delivery module discussed above to move within the housing and eject the solid dosage form as a projectile from the cartridge into the GI lumen wall or surrounding tissue of the subject. The solid dosage form may degrade in the GI lumen wall or surrounding tissue to release the therapeutic agent(s) for systemic uptake.

In this way, the disclosed ingestible devices can autonomously deliver different dosage forms and/or dosage amounts from different delivery mechanisms using a common device platform.

As discussed below, the disclosed devices and assemblies can deliver (locally or systemically) a variety of different types of therapeutic agents, such as biologics including macromolecules that are normally unsuitable for delivery by ingestion to a subject. The disclosed devices and assemblies are advantageously structured to deliver different dosage forms that may include different types of therapeutic agents regardless of molecular size or weight, such as small molecules, anti-virals, anti-infectives, peptides, polypeptides, proteins, hormones, antibodies, and nucleic acids. For example, and without limitation, the devices and assemblies disclosed herein can deliver one or more therapeutic agents such as an immunosuppressive drug (e.g., adalimumab, ustekinumab), a chemotherapy drug, a central nervous system (CNS) drug (e.g., antiparkinson agents, antiemetic agents), an antidiabetic drug, an enzyme replacement therapy (ERT) drug, a PD-L1 drug, a CNP drug, an antibody (e.g., nanobodies, monoclonal antibodies, anti-TNFα antibodies, anti-interleukin antibodies (e.g., targeting one or more of IL 1-36, such as IL-2, IL-4, IL-12, IL-13, IL-17, IL-23), anti-PCSK9 antibodies, anti-TL1A antibodies, antibody drug conjugates (ADCs), bispecific antibodies, and analogues thereof), a hormone (e.g., parathyroid hormone (PTH), follicle stimulating hormone (FSH), human growth hormone (HGH), amylin, and analogues thereof), an anti-coagulant or blood clotting factor (e.g., Factor VIII, Factor XI, Factor X, or mimetics thereof), insulin, an incretin or a combination thereof (e.g., one or more of glucagon-like peptides (e.g., GLP-1, GLP-2), gastric inhibitory peptide (GIP), glucagon, glucagon receptor (GCGR), peptide YY (PYY), glucose-dependent insulinotropic polypeptide (GIP), GIP receptor (GIPR), and mimetics thereof), an oligonucleotide (e.g., antisense oligonucleotide (ASO), RNA interference (RNAi), aptamer RNAs), a DNA or SiRNA transcript, a cell, a cytotoxic agent, a vaccine or other prophylactic agent, a nutraceutical agent, a vasodilator, or a vasoconstrictor, a delivery enhancing agent, a delay agent, an excipient, a diagnostic agent, or a substance for cosmetic enhancement.

The disclosed ingestible devices and assemblies can be used to autonomously deliver dosage forms including one or more therapeutic agents to provide treatment for a number of medical conditions and diseases. Examples of medical conditions and diseases which can be treated can include, without limitation: cancer (including lung cancer, pancreatic cancer, cervical cancer, gastric cancer, breast cancer, glioblastoma, colorectal cancer, multiple myeloma, leukemia, lymphomas, carcinomas, and melanomas), neutropenia, multiple sclerosis (MS), HIV, short bowel syndrome, mucopolysaccharidosis, hormonal conditions (e.g., hypo/hyper thyroid, growth hormone conditions, fertility conditions), osteoporosis, metabolic disorders (including high blood pressure, elevated cholesterol and triglyceride, diabetes and other glucose regulation disorders, and weight disorders (e.g., obesity)), infection (local or septicemia), epilepsy and other seizure disorders, CNS disorders (e.g., migraine prevention), coronary arrhythmia (both atrial and ventricular), coronary ischemia anemia, various autoimmune disorders (including multiple sclerosis, Guillain-Barre syndrome, rheumatoid arthritis, ankylosing spondylitis, psoriasis, psoriatic arthritis, Crohn's disease, ulcerative colitis, hidradenitis suppurativa, juvenile idiopathic arthritis, uveitis, chronic inflammatory demyelinating polyneuropathy, multifocal motor neuropathy, and lupus), allergic diseases (e.g., eczema, asthma, and chronic rhinosinusitis with nasal polyposis), eosinophilic esophagitis, prurigo nodularis, coagulation disorders (e.g., hemophilia, Von Willebrand disease, clotting factor deficiencies, hypercoagulable states, and deep venous thrombosis), and other conditions.

Non-limiting examples of specific therapeutic agents, approved dosages, and associated indications that could be treated using the disclosed ingestible devices are listed in Table 1 below. Generally speaking, without being limiting, the listed therapeutic agents that have an equivalent daily dose of less than about 5 mg may be reformulated into a solid dosage form for delivery using the ingestible devices as described herein that are adapted for delivery of a solid dosage form. The listed therapeutic agents that have an equivalent daily dose of less than about 60 mg may be delivered as a liquid dosage form without requiring reformulation using the ingestible devices described herein that are adapted for delivery of a fluid dosage form.

Fluid dosage forms discussed herein may include a liquid, a slurry, a gel, a suspension (including a colloidal suspension), a gas, a powder, or any combination thereof. The disclosed devices and assemblies can deliver fluid dosage forms that are used or approved for parenteral administration (e.g., subcutaneous, intravenous, or intraperitoneal injection) without requiring reformulation or alteration of the fluid dosage form.

Solid dosage forms discussed herein may include a tablet, a microneedle, a slug, or other shaped composition. For example, solid dosage forms may be formed by compression of a lyophilized powder containing one or more therapeutic agents and one or more additional components (e.g., an excipient, a binder, a preservative, a lubricant). The compression process may be performed in such a way to substantially preserve the bioactivity of the active therapeutic agent in the compressed solid dosage form. Examples of suitable solid dosage forms and a related formation process are described in U.S. Pat. No. 10,098,931 (and its related progeny), entitled, “PHARMACEUTICAL COMPOSITIONS AND METHODS FOR FABRICATION OF SOLID MASSES COMPRISING IMMUNOGLOBULINS”, the entire contents of which are hereby incorporated by reference herein.

In one or more embodiments, the solid dosage form may include a separate biodegradable structure for insertion into a GI lumen wall or surrounding tissue, which biodegradable structure may degrade in vivo to release one or more therapeutic agents contained therein. For example, the biodegradable structure may have a needle, arrow, hook, or other tapered/pointed structure sufficient to penetrate a GI lumen wall or surrounding tissue. The biodegradable structure may further define a cavity or reservoir for containing a compressed tablet or other composition (e.g., a gel, a liquid) including the therapeutic agent(s) for delivery into the GI lumen wall or surrounding tissue.

As described in more detail below, the disclosed devices and assemblies may be structured to deliver a dosage form into a layer of, or through, the GI lumen wall and into the surrounding tissue thereof, such as into the peritoneum or peritoneal cavity of a subject. Oral delivery into the peritoneal space may be particularly advantageous for the efficient uptake of a variety of therapeutic agents, such as, for example, CNS drugs, immunosuppressive drugs, oligonucleotides, antibodies, hormones, blood clotting factors, and many other types of therapeutic agents such as those described in Table 1 herein, while avoiding the pain and inconvenience associated with parenteral administration.

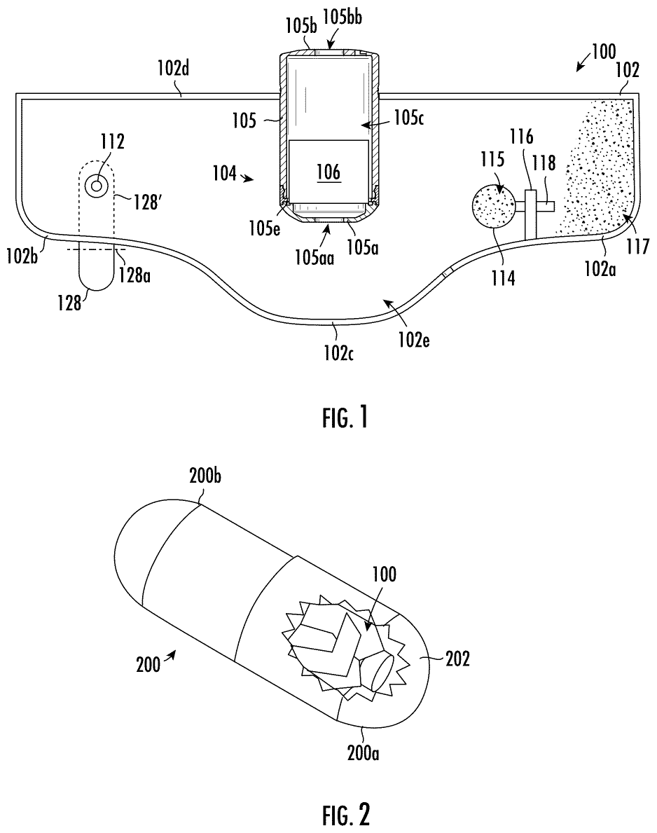

FIG. 1 illustrates an example of an ingestible device 100 according to one or more embodiments of the present disclosure. Device 100 includes an expandable member in the form of a balloon 102 and a delivery assembly 104 coupled to balloon 102. In other examples, device 100 may include an expandable member in the form of an accordion shaped structure, foldable wings, a patch, or other expandable structure.

It should be appreciated, however, that the embodiments of delivery assembly 104 and the payload delivery modules described herein may be used with, or otherwise incorporated into, a variety of different device configurations other than those described herein. For example, embodiments of delivery assembly 104 may be incorporated directly into a swallowable capsule, endoscope, or other ingestible enclosure, which enclosure may include a force generating mechanism operatively coupled to delivery assembly 104. The force generating mechanism may include one or more of a spring, a gas generating mechanism, or other mechanism for generating a force within the ingestible enclosure. The generated force may be used to actuate delivery assembly 104 to deliver a dosage form through one or more openings in the enclosure into a GI lumen wall or surrounding tissue thereof. The openings may be temporarily covered by a coating, plug, shell, or other structure that can degrade in response to a condition in the GI tract to reveal the opening(s), such as in the presence of GI fluid or in response to a particular pH in the GI tract (e.g., a pH in the small intestine).

As described herein, in one or more embodiments, delivery assembly 104 can have a modular design structured with a common release feature or trigger to be able to incorporate different payload delivery modules 106 (shown schematically in FIG. 1) for the oral delivery of different dosage forms and/or dosage amounts into a GI lumen wall or surrounding tissue of a subject after ingestion of device 100. The different payload delivery modules 106 may each have a different delivery mode to provide design flexibility and to facilitate the delivery of different dosage forms and dosage amounts.

As shown in FIG. 1, device 100 is in an unfolded state, but it should be appreciated that device 100 may be folded (e.g., rolled, flattened, twisted) in such a manner to fit inside an ingestible enclosure to facilitate ingestion of device 100. For example, an ingestible enclosure may include a swallowable capsule (e.g., a size 00, size 000, or other size capsule), an endoscope, or other enclosure that is suitable for ingestion by a subject.

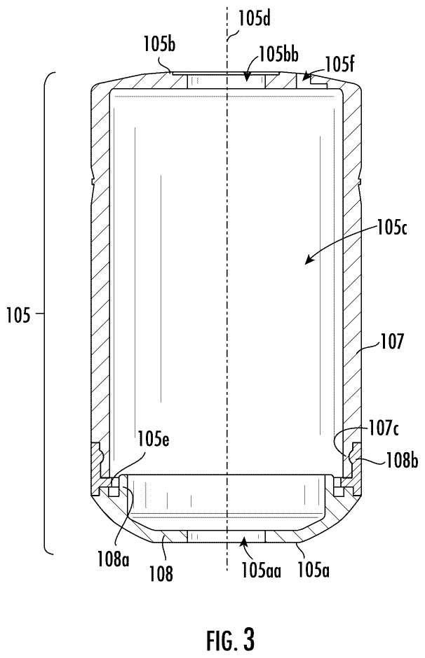

FIG. 2 illustrates device 100 in a folded state and disposed in an ingestible enclosure shown as a swallowable capsule 200 according to one example. Capsule 200 includes an optional outer coating 202 disposed over at least an outer portion of capsule 200. Capsule 200 and outer coating 202, if present, are structured to allow for ingestion of device 100, and to temporarily protect the contents of device 100 from degradation within one or more portions of the GI tract of a subject. As shown in FIG. 2, capsule 200 includes a first section 200a at least partially overlapping a second section 200b in a press-fit arrangement to define capsule 200. The first and second sections 200a, 200b may be detachably coupled together to allow for separation of the two sections.

In one or more embodiments, capsule 200 can degrade under certain conditions. Further, different portions of capsule 200 may be structured to degrade under different conditions or at different degradation rates depending on a target site within the GI tract for delivering a dosage form. For example, a portion of, or all of, capsule 200 may be constructed of a material that degrades in water (e.g., in the presence of water in the form of humidity or moisture in an ambient environment, such as within the body) and/or degrades when exposed to a pH level above a particular threshold or within a particular range (e.g., a pH level associated with a desired location or portion of the GI tract, such as a pH level associated with a target site within the GI tract (e.g., stomach, small intestine) for delivering a dosage form). For example, capsule 200 may be formed from, or include, hydroxypropyl methylcellulose (HPMC) or other biodegradable material or combinations of materials.

Optional outer coating 202 optionally covers a portion of, or all of, capsule 200. Outer coating 202 may include a single layer or multiple layers. The various layers may be formed of the same material or different materials. In one or more embodiments, outer coating 202 can degrade under certain conditions, as described above with reference to enclosure 200. An example of outer coating 202 is an enteric coating, such as an enteric coating that degrades in water at a given rate and/or degrades when exposed to solutions with a pH level above a particular threshold or within a particular range. Another example of outer coating 202 is a protective coating (e.g., wax), such as a coating which protects a portion of an outer surface of capsule 200 from coming into contact with fluids or tissues (e.g., bodily tissue or fluids).

In one or more embodiments, degradation of capsule 200 and/or outer coating 202, if present, can allow fluid (e.g., bodily fluid in the stomach or in the intestine) to enter an interior of capsule 200 to cause balloon 102 to expand, as described in further detail herein. Capsule 200 and/or outer coating 202, if present, may define one or more degradation areas for localized degradation to, for example, allow for controlled degradation and separation of capsule 200. For example, outer coating 202 may be selectively applied only to certain areas of capsule 200 (e.g., on the hemispherical ends of capsule 200) to expose a selected portion of capsule 200 (e.g., a middle portion of capsule 200 between the ends), thereby defining an area of capsule 200 that can degrade at a faster rate and/or degrade sooner than other areas of capsule 200. This controlled degradation of capsule 200 may allow for more consistent separation of capsule 200 with a GI lumen to thereby allow for the delivery of various dosage forms into the GI lumen wall or surrounding tissue thereof.

Referring again to FIG. 1, balloon 102 is a flexible and adjustable structure that can expand from a collapsed state (e.g., folded) to an expanded state within a desired location of a GI tract of a subject for delivery of a dosage form into the GI lumen wall or surrounding tissue thereof. Balloon may be a monolithic structure or may be composed of one or more sections that are coupled (e.g., sealed) together. Balloon 102 may be formed from or include one or more layers of a material, such as a sheet-like material. Suitable materials for balloon 102 can include, for example, hydroxypropyl methylcellulose (HPMC), polyvinyl acetate (PVA), polyethylene, lactide, glycolide, lactic acid, glycolic acid, par-dioxanone, trimethylene carbonate, caprolactone, and mixtures and copolymers thereof.

As shown in FIG. 1, balloon 102 includes an inflator section 102a, a deflator section 102b, a lower section 102c, and an elongated section 102d extending between inflator section 102a and deflator section 102b. Lower section 102c extends downward (in the orientation shown in FIG. 1) between inflator section 102a and deflator section 102b. The various sections of balloon 102 cooperatively define an enclosed interior 102e for containing various components of device 100. As discussed below, balloon 102 is structured to inflate at a desired location within a lumen of the GI tract (e.g., stomach, small intestine, large intestine) in response to a gas pressure generated within interior 102e via a gas generating mechanism coupled to balloon interior 102e.

Balloon 102 and the gas generating mechanism can cooperatively define an expandable member assembly of device 100. In this way, and as described herein, balloon 102 can facilitate delivery of different dosage forms and dosage amounts from delivery assembly 104 into the GI lumen wall or surrounding tissue thereof.

In other examples, balloon 102, or other expandable member, may be self-expandable (e.g., using a shape memory material, such as a spring or wireframe structure held in a compressed position) or may include a separate force generating mechanism to cause expansion. For example, the force generating mechanism may include any one of, or a combination of, a gas generating mechanism, a hydrogel, a pre-loaded (e.g., compressed) spring, a combustible substance (e.g., nitrocellulose), or other mechanism structured to generate a sufficient force to cause expansion of balloon 102 within the GI tract of a subject.

Balloon 102 has a size and shape to occupy a space in a GI lumen upon inflation of balloon 102 to help facilitate delivery of a dosage form into the GI lumen wall or surrounding tissue. For example, upon inflation of balloon 102 in a GI lumen, an outer periphery of balloon 102 (e.g., an outer periphery of lower section 102c and elongated section 102d) pushes against a surface of the lumen wall. The pressure exerted by balloon 102 is sufficient to temporarily hold balloon 102 (and in turn, delivery assembly 104) relative to the lumen wall for delivery of the dosage form. Depending on an inner circumference of the lumen delivery site, lower section 102d may remain partially folded, or may extend fully, when balloon 102 is inflated. For example, if the lumen is relatively large and there is no obstruction to resist expansion of balloon 102, then balloon 102 would assume a fully inflated configuration with lower section 102c fully extended (as shown in FIG. 1). If, however, the lumen is relatively small such that the inner lumen circumference is less than a maximum dimension of the fully inflated balloon 102, then lower section 106 would remain partially folded. In this way, balloon 102 can self-adjust to the size of a GI lumen to hold balloon 102 in position for delivery of the dosage form, such that the same balloon 102 can be used for a broad range of GI lumen sizes (e.g., different inner circumferences).

Balloon 102 includes a deflation valve 112 structured to cause deflation of balloon 102 upon completing delivery of a dosage form into the GI lumen wall or surrounding tissue thereof. In this way, deflation valve 112 can facilitate passage of balloon 102 through the remainder of the GI tract to exit the anus of the subject. In the example shown, deflation valve 112 is structured as a degradable plug which temporarily covers an opening leading into interior 102e. The degradable plug may be structured to degrade in response to contact with fluid in the GI tract (e.g., bodily fluid) to thereby allow gas contained in interior 102e to exit through the opening. For example, deflation valve 112 may be formed from, or include, an enteric material. Balloon 102 may include an optional flap 128 which can temporarily cover deflation valve 112 until balloon 102 is inflated to thereby prevent premature activation (e.g., degradation) of deflation valve 112. For example, flap 128 may be temporarily held (e.g., adhered, tacked, or otherwise held) in a folded position 128′ about a flap folding axis 128a. The expansion of balloon 102 can cause flap 128 to unfold from the folded position 128′ to expose deflation valve 112 and allow fluid in the GI tract to reach deflation valve 112 and cause its degradation, thereby providing an opening for gas to exit through to permit deflation.

Deflation valve 112 is shown located on deflator section 102b, but deflation valve 112 may be located elsewhere on balloon 102 according to other embodiments. Further, balloon 102 may include more than one deflation valve 112. Deflation valve 112 may be structured differently than the example shown in FIG. 1. For example, in other embodiments, deflation valve 112 may be structured as a degradable and/or movable cover disposed over an opening on balloon 102. In these embodiments, degradation and/or movement of the cover away from the opening can cause gas to exit from interior 102e through the opening, thereby permitting deflation.

Balloon 102 further includes a gas generating mechanism including a reactant reservoir 114 that is disposed within, or otherwise coupled to, balloon interior 102e. Reactant reservoir 114 is structured to hold a first reactant 115 therein and to temporarily prevent first reactant 115 from contacting a second reactant 117, which is separately disposed within balloon interior 102e. First reactant 115 may be, for example, citric acid. Second reactant 117 may be, for example, a carbonate, such as potassium bicarbonate. In other examples, first reactant 115 and second reactant 117 may be other types of reactants (e.g., an acid and a base) which when mixed result in the formation of a gas sufficient to inflate balloon 102. Second reactant 117 is shown disposed within balloon interior 102e at inflator section 102a near first reactant 115. In other examples, first reactant 115 and second reactant 117 may be contained in other areas of balloon 102 so long as they are temporarily separated from each other.

In other examples, balloon 102 may include a different type of gas generating mechanism, such as a pressurized gas container (e.g., carbon dioxide (CO₂) cartridge), or other type of gas generating mechanism that can be disposed in, or otherwise coupled to (e.g., fluidly coupled), balloon 102 or other expandable member.

Still referring to FIG. 1, reactant reservoir 114 defines an interior volume for containing first reactant 115. Reactant reservoir 114 may take a variety of different forms and shapes, such as a balloon, bladder, container, or other structure. Reactant reservoir 114 may include, or be formed from, a flexible polymeric material to allow for elastic deformation. Reactant reservoir 114 is in selective fluid communication with balloon interior 102e via a reactant conduit 118 and a release 116. Release 116 is coupled to reactant conduit 118 on an outer portion of balloon 102 such that upon activation (e.g., degradation) of release 116, first reactant 115 can exit from reactant reservoir 114 into balloon interior 102e via reactant conduit 118. For example, reactant reservoir 114 may be pressurized by a volume of first reactant 115 disposed therein. Release 116 may be in the form of a degradable plug or clip which blocks an interior portion of reactant conduit 118 to temporarily prevent first reactant 115 from entering interior 102e from reservoir 114. When fluid in the GI tract contacts release 116 (e.g., upon degradation of capsule 200 and/or outer coating 202), release 116 can subsequently degrade to allow reactant reservoir 114 to discharge first reactant 115 via reactant conduit 118 into balloon interior 102e.

In other examples, device 100 may include a clip, a band, or other structure for holding a portion of balloon 102 in such a manner to temporarily define separate chambers within balloon interior 102e for separately containing first reactant 115 and second reactant 117, respectively. For example, a portion of balloon 102 may be pinched or compressed by a degradable clip or band to temporarily define the separate chambers within balloon interior 102e. The chambers may be substantially sealed from each other to substantially prevent first reactant 115 and second reactant 117 from mixing. The clip or band may be located on an outer portion of balloon 102 such that exposure to fluid in the GI tract (e.g., upon degradation of capsule 200 and/or outer coating 202) can cause degradation of the clip or band and subsequent release from balloon 102. Upon release of the clip or band or like structure from balloon 102, the separate chambers are no longer substantially sealed from each other thereby allowing first reactant 115 to mix with second reactant 117 within balloon interior 102e.

Combining first reactant 115 with second reactant 117 within interior 102e causes a chemical reaction resulting in the formation of a gas (e.g., CO₂). The gas causes balloon 102 to expand to an inflated state within a GI lumen resulting in substantial alignment of elongated section 102d with a surface of the GI lumen wall, which thereby results in substantially perpendicular alignment of a longitudinal axis of delivery assembly 104 with the lumen wall surface. Further, expansion of balloon 102 within a GI lumen can position delivery assembly 104 relative to (e.g., proximate to or in contact with) a surface of the GI lumen wall. Substantial alignment of elongated section 102d and positioning/orienting of delivery assembly 104 relative to the GI lumen wall can, advantageously, help to facilitate delivery of a dosage form from delivery assembly 104 into the GI lumen wall or surrounding tissue thereof without exposing the dosage form to the GI lumen environment. In this manner, device 100 can substantially preserve the dosage form within a portion of the GI tract before delivery into a GI lumen wall or surrounding tissue for systemic uptake of one or more therapeutic agents contained therein.

Still referring to FIG. 1, delivery assembly 104 is coupled to balloon 102 and is at least partially or fully disposed within balloon interior 102e. In other examples, delivery assembly 104 can be positioned entirely outside of balloon interior 102e but be in fluid communication with balloon interior 102e. Delivery assembly 104 is shown to include a housing 105 and a payload delivery module 106. Payload delivery module 106 is shown schematically in FIG. 1 to illustrate that housing 105 may have a modular construction to accommodate different payload delivery modules 106 for the delivery of different dosage forms and/or dosage amounts, as described in further detail herein.

As shown in FIG. 1, housing 105 has a generally hollow, cylindrical shape and includes a housing proximal end 105a, a housing distal end 105b, and a housing interior 105c located between housing proximal end 105a and housing distal end 105b. Housing 105 has a longitudinal axis 105d extending between housing proximal end 105a and housing distal end 105b. Housing proximal end 105a has a first opening 105aa for receiving a force (e.g., a gas pressure from a gas generating mechanism) within housing interior 105c. Housing distal end 105b has a second opening 105bb for discharging a dosage form from housing interior 105c into a GI lumen wall or surrounding tissue thereof. Housing distal end 105b also may have an optional vent opening 105f extending to housing interior 105c to function as a pressure relief for housing interior 105c and balloon interior 102e. Housing interior 105c defines a receptacle or piston chamber for receiving payload delivery module 106 therein. Housing 105 further includes a housing release 105e (e.g., release feature, trigger) located in housing interior 105c. Housing release 105e extends laterally inward toward longitudinal axis 105d and away from a side wall of housing 105, which side wall defines the receptacle or piston chamber of housing 105. As described herein, housing release 105e can be structured to releasably couple different embodiments of payload delivery module 106 within housing interior 105c. For example, and as described in further detail below, housing release 105e is structured to release payload delivery module 106 from housing 105 in response to a threshold force applied against payload delivery module 106 through first opening 105aa to thereby cause axial movement of payload delivery module 106 within housing interior 105c between housing proximal end 105a and housing distal end 105b to discharge the dosage form from housing interior 105c through second opening 105bb.

By positioning housing release 105e laterally on housing 105, the longitudinal space within housing interior 105c may be optimized to, for example, contain a maximum dosage in payload delivery module 106. In other words, housing interior 105c is substantially unobstructed along longitudinal axis 105d because of the lateral position of housing release 105e. This may be particularly advantageous to maximize the dosage amount within interior 105c without requiring an increase in size of housing 105 or balloon 102 and thereby an increase in size of the ingestible enclosure to orally deliver device 100.

According to other examples, housing release 105e may be positioned at a different location on housing 105. For example, housing release 105e may be positioned at housing proximal end 105a or at housing distal end 105b.

Referring now to FIG. 3, housing 105 is shown separately from balloon 102 without payload delivery module 106 disposed therein. As shown in FIG. 3, housing 105 includes a body 107 and a cover 108 (which includes housing release 105e). In another example, housing 105 may be a unitary, one-piece structure with an integrated release feature (e.g., a release feature defined by a side wall of body 107) without a separate cover.

Body 107 includes housing distal end 105b, second opening 105bb, housing interior 105c, and optional vent opening 105f. Body 107 has a longitudinal axis 105d. Body 107 is structured to be coupled (e.g., sealed, adhered) to balloon 102 along elongated portion 102d. For example, a portion of housing 105 may be disposed through an opening of balloon 102 in balloon interior 102e. Elongated portion 102d of balloon 102 may overlap with a portion of an outer surface of body 107 such that the overlapping portion can be heat sealed to body 107 to thereby couple housing 105 to balloon 102. Body 107 further includes a cover attachment feature 107c for coupling cover 108 thereto.

Cover 108 is coupled (e.g., press-fit, snap fit, adhered, ultrasonically welded) to body 107 via cover attachment feature 107c and a complementary feature 108b on cover 108. In the example shown in FIG. 3, cover 108 is snap-fit to body 107. Cover 108 includes housing release 105e, housing proximal end 105a, and first opening 105aa. In other examples, housing release 105e may be defined by a separate component that is coupled (e.g., adhered, heat sealed) to, or integrally formed with, cover 108. In the example shown, housing release 105e is generally ring shaped and is structured as a press-fit feature for releasably coupling payload delivery module 106 to body 107. Specifically, in the example shown, housing release 105e is structured as a protrusion that extends laterally inward from a side wall of body 107 toward longitudinal axis 105d. Cover 108 further includes an inner wall 108a that can function as a guide or stabilizer during at least a portion of the axial travel of payload delivery module 106, as described in further detail herein.

According to other examples, housing release 105e may be structured differently than the example of FIGS. 1 and 3, such as another type of press-fit feature (e.g., a groove or channel, flexible fingers), a frangible connection, or other type of feature that is structured to release (e.g., deflect, break, move) payload delivery module 106 at or above a threshold force received through first opening 105aa.

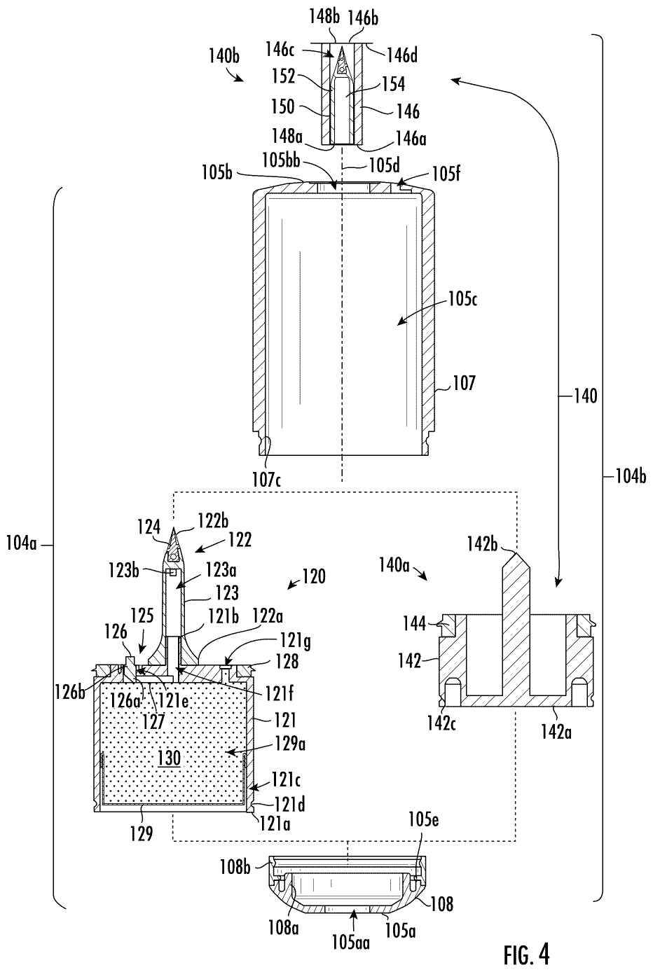

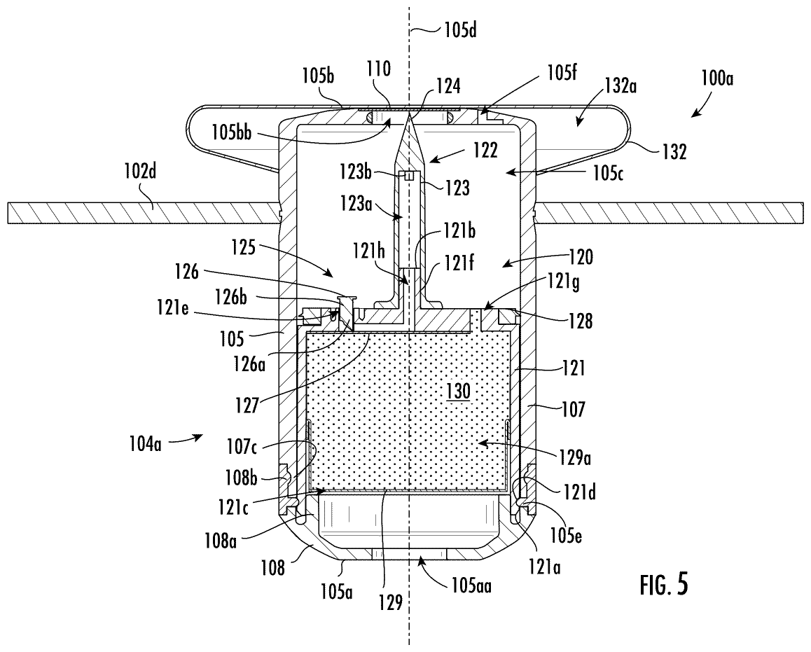

Referring now to FIG. 4, an exploded view of two different modular configurations of delivery assembly 104 is shown according to an embodiment. A first configuration of delivery assembly 104, shown as a first modular delivery assembly 104a on the left side of FIG. 4, includes housing 105 (e.g., body 107 and cover 108) and a first payload delivery module 120 (an embodiment of payload delivery module 106) having a fluid dosage form 130 disposed therein. First payload delivery module 120 is structured to be releasably coupled to, and movably disposed in, housing 105.

According to an example assembly process, cover 108 is releasably coupled to first payload delivery module 120 via housing release 105e in a first assembly step, which may occur prior to filling payload delivery module 120 with fluid dosage form 130, as described herein. Next, the sub-assembly of cover 108 and first payload delivery module 120 is coupled to body 107 via cover attachment feature 107c with first payload delivery module 120 disposed in housing interior 105c. The delivery assembly including housing 105 and first payload delivery module 120 can then be subsequently coupled to balloon 102.

Still referring to FIG. 4, a second, alternative configuration, shown as a second modular delivery assembly 104b on the right side of FIG. 4, includes housing 105 and a second payload delivery module 140 (another embodiment of payload delivery module 106) having a solid dosage form 150. As shown in FIG. 4, second payload delivery module 140 is collectively defined by an ejector 140a and a cartridge 140b. Ejector 140a is structured to be releasably coupled to, and slidably disposed in, housing 105. Cartridge 140b is structured to contain solid dosage form 150 therein and to be coupled (e.g., press-fit, snap-fit, adhered) to housing 105 at housing distal end 105b.

According to an example assembly process, cover 108 is releasably coupled to ejector 140a via housing release 105e in a first assembly step. Next, the sub-assembly of cover 108 and ejector 140a is coupled to body 107 via cover attachment feature 107c with ejector 140a disposed in housing interior 105c. In a third step, cartridge 140b is coupled to housing 105 at housing distal end 105b. Alternatively, cartridge 140b may be coupled to housing 105 before or simultaneously with coupling the sub-assembly of cover 108 and ejector 140a to body 107.

As represented by dotted lines in the example of FIG. 4, housing 105 can be advantageously structured to receive either first payload delivery module 120 or second payload delivery module 140 therein using a common release feature or trigger (e.g., housing release 105e). In this manner, housing 105, and in turn device 100, can accommodate different delivery mechanisms or modules with different delivery modes to enable the delivery of different dosage forms and/or dosage amounts.

Still referring to FIG. 4, first payload delivery module 120 includes a piston 121, a needle 122, a valve 125, and a membrane 129. In other examples, at least a portion of valve 125 may be coupled to, or form part of, housing 105 and may be structured to interface with piston 121 to control a flow of fluid dosage form 130, as described herein.

Piston 121 is structured to releasably couple first payload delivery module 120 to housing 105. Piston 121 is further structured to selectively move (e.g., slide) within housing interior 121c to advance needle 122 from housing 105 into a GI lumen wall or surrounding tissue thereof. As shown in FIG. 4, piston 121 has a generally hollow cylindrical shape and has a piston proximal end 121a and a piston distal end 121b. Piston 121 has a piston interior 121c for containing fluid dosage form 130. Piston proximal end 121a is open to piston interior 121c to allow for a force (e.g., a gas pressure) to be applied against a surface of membrane 129, as discussed in further detail herein. Piston distal end 121b is defined by a needle port 121f for coupling needle 122 to piston 121. Needle port 121f defines a piston fluid channel 121h for fluid dosage form 130 to flow through to needle 122. An upper wall of piston 121 adjacent to piston distal end 121b includes a valve opening 121e for receiving part of valve 125 therein. The upper wall further includes a fill port 121g for filling a reservoir defined, in part, by piston interior 121c with fluid dosage form 130 as described herein.

Still referring to FIG. 4, a lateral wall (e.g., an outer circumferential surface) of piston 121 includes a piston release 121d (e.g., release feature, trigger) that is complementary to housing release 105e for releasably coupling first payload delivery module 120 to housing 105. In the example shown, piston release 121d is structured as a channel that is complementary to the protrusion of housing release 105e to define a press-fit arrangement, although other structural configurations are contemplated (e.g., a protrusion, a frangible connection) depending on the structure of housing release 105e. As described below, piston release 121d is structured to release from housing release 105e in response to a force applied to piston 121 through first opening 105aa reaching or exceeding a threshold value to cause piston 121 to move axially within housing interior 105c toward the GI lumen wall. For example, the force may be a threshold gas pressure generated within balloon interior 102e by a gas generating mechanism (e.g., mixing of first reactant 115 with second reactant 117). In this example, the threshold gas pressure may be in a range of about 10-40 psi, including from about 20-40 psi, such as about 20 psi, 25 psi, 30 psi, 35 psi, 40 psi, or any value therebetween.

In other examples, the force may be a mechanical or a fluidic force generated by a separate force generating mechanism operably coupled to piston 121. For example, the force generating mechanism may be a spring, a hydrogel, a gas source, or other mechanism for generating a force to apply against piston 121.

An optional piston seal 128 is coupled to piston 121 along a lateral wall of piston 121. Piston seal 126 is structured to create a substantially fluid tight seal between piston 121 and housing 105 to, for example, substantially prevent gas within balloon interior 102e from passing between piston 121 and housing 105. Piston seal 128 is further structured to allow relative (sliding) movement between piston 121 and housing 105.

Needle 122 is coupled to piston 121 at piston distal end 121b via needle port 121f. Needle 122 has a generally elongated structure. In the example shown, needle 122 is collectively defined by a needle body 123 and a needle tip 124. In other examples, needle 122 may be a monolithic one-piece structure and/or may have other structural configurations, such as a hook, arrow, or other tissue piercing structure. As shown in FIG. 4, needle body 123 has a generally hollow cylindrical shape with a needle fluid channel 123a that extends longitudinally from a proximal end to a discharge opening 123b. Discharge opening 123b extends through a side wall of needle body 123.

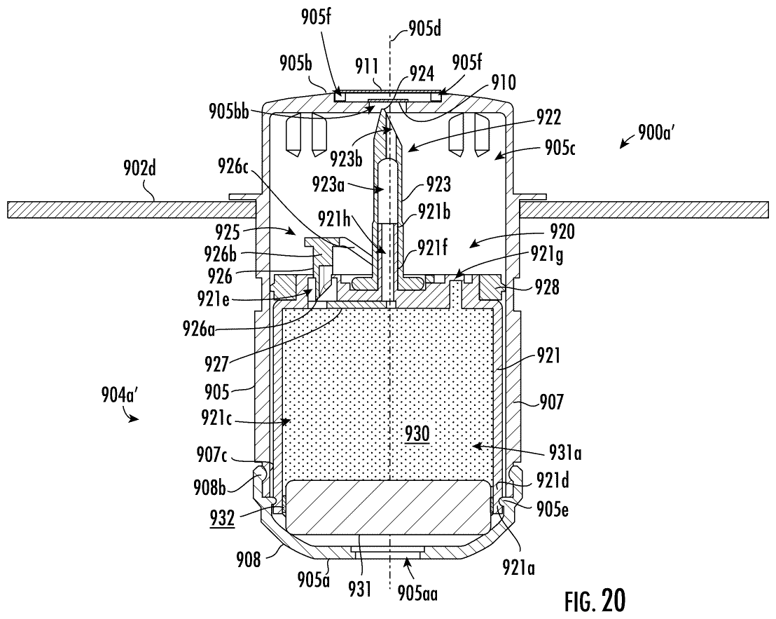

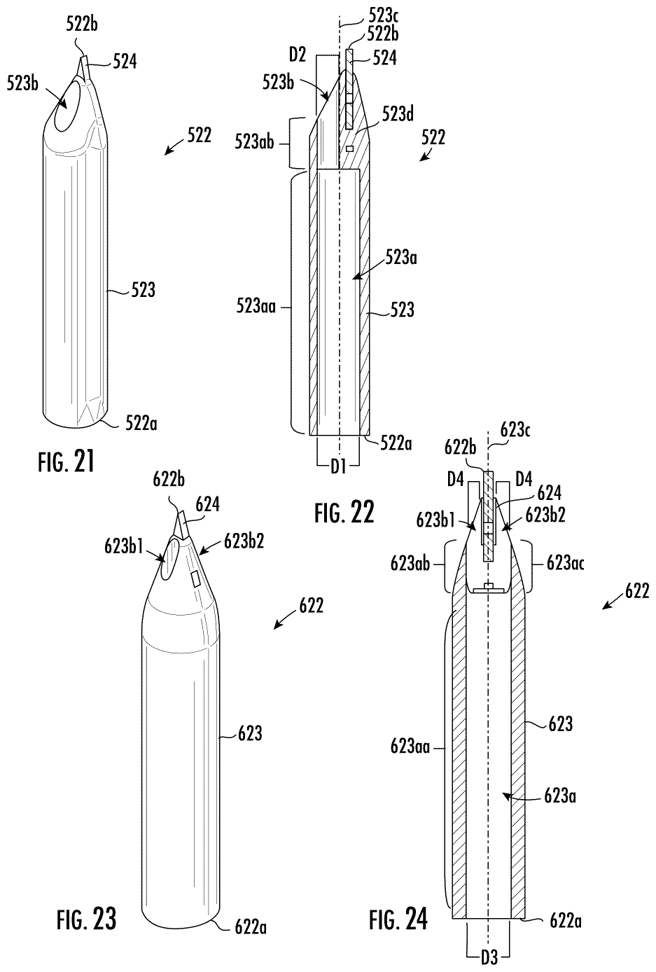

In other examples, such as the examples shown in FIGS. 21-24 discussed below, needle body 123 may have a plurality of fluid channels and/or discharge openings. Further, needle fluid channel 123a may extend through needle body 123 at a distal end thereof instead of a side wall of needle body 123.

Needle body 123 may be formed from or include a biodegradable material to allow for degradation of at least a portion, or all, of needle body 123 in situ upon delivery of fluid dosage form 130. For example, needle body 123 may be formed from or include polyethylene oxide (PEO), polyethylene glycol (PEG), polyglycolide-co-lactide (PGLA), or other material or combinations of materials. In other examples, needle body 123 may be formed from or include a substantially non-degradable material, such as a surgical grade steel, a polymeric material, or other material or combinations of materials. In any of these examples of needle 122, payload delivery module 120 and/or housing 105 may include a biasing component (e.g., a spring) or other mechanism to cause retraction of needle 122 from tissue into housing interior 105c upon delivery of fluid dosage form 130. For example, the biasing component may have a sufficient biasing force to cause retraction of needle 122 from tissue upon the gas pressure within balloon 102 decreasing below a threshold value, such as a threshold gas pressure within balloon 102 shortly after delivering fluid dosage form 130.

Needle tip 124 is coupled to (e.g., press-fit, adhered) or integrally formed (e.g., insert molded) with needle body 123. Needle tip 124 has a tapered or pointed shape sufficient to penetrate a GI lumen wall or surrounding tissue thereof. Needle tip 124 may be formed from or include a biodegradable material or a combination of materials, such as magnesium, PEO, PEG, or other material. Needle tip 124 may be formed from a material that is harder than a material of needle body 123 to help facilitate penetration of needle body 123 into the GI lumen wall or surrounding tissue. For example, needle tip 124 may be formed from a surgical grade steel, magnesium, or other material, whereas needle body 123 may be formed from PEO, PEG, or other relatively softer material.

To provide additional design flexibility, needle 122 may have a length sufficient to penetrate to a desired penetration depth, such as into a layer of the GI lumen wall or through the GI lumen wall into the surrounding tissue thereof (e.g., peritoneum or peritoneal cavity) of a subject for delivery of fluid dosage form 130. Applicant advantageously determined that needle penetration depth is a function of the location of the device in the GI tract for delivery, as the inner circumference of the GI lumen varies along its length, the thickness of the GI wall in an unstretched state, and the amount of GI lumen wall stretch resulting from expansion of expandable member 102, which can change the local wall thickness and is dependent upon the inner circumference of the GI lumen.

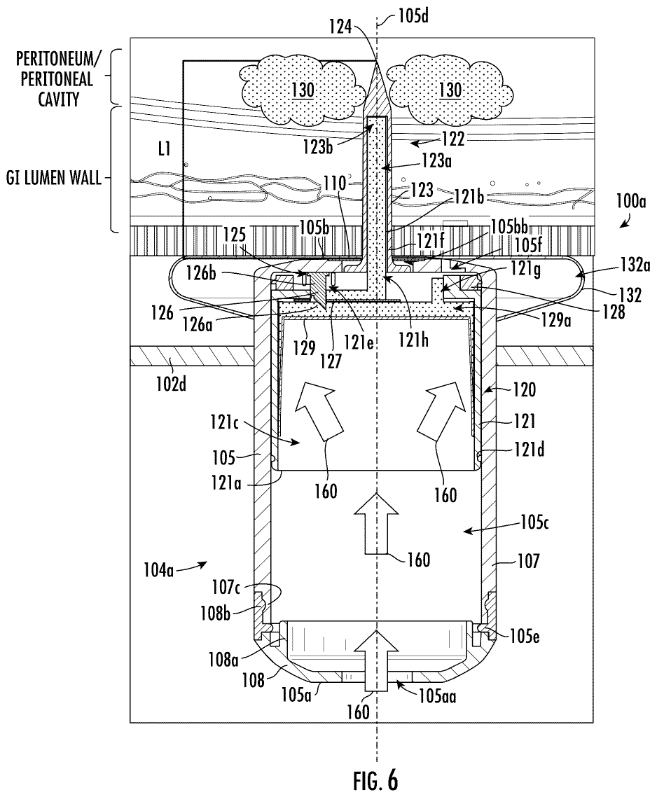

Based on these factors, needle 122 may have a critical length L1 (shown in FIG. 6 and defined by a distance between distal end 105b of housing 105 and needle tip 124 upon inflation of balloon 102 and subsequent advancement of piston 121 within housing interior 105c) in a range of about 3.5-12 mm to penetrate into and through the GI lumen wall (e.g., through the serosa of the small intestine) into the peritoneal cavity of a subject for discharging fluid dosage form 130 therein.

In other examples, needle 122 may have a critical length L1 in a range of about 1.5-3 mm to penetrate into a layer of the GI lumen wall, such as a submucosal layer, for discharging fluid dosage form 130 therein.

Referring now to FIGS. 21-24, two alternative examples of needle 122 are illustrated. In a first alternative example shown in FIGS. 21-22, a needle 522 is illustrated with a discharge opening 523b located at or near a distal end of needle 522. In particular, needle 522 includes a needle body 523 that has a needle fluid channel 523a extending longitudinally from a needle proximal end 522a to a discharge opening 523b located at or near a needle distal end 522b. Needle fluid channel 523a includes a first fluid channel section 523aa and a second fluid channel section 523ab. First fluid channel section 523aa extends from needle proximal end 522a and has a first diameter D1 that is centered along a longitudinal axis 523c of needle body 523. Second fluid channel section 523ab extends from first fluid channel section 523a to discharge opening 523b at or near needle distal end 522b. Second fluid channel section 523ab has a second diameter D2 that is less than first diameter D1 of first fluid channel section 523aa. Further, second fluid channel section 523ab is positioned offset from longitudinal axis 523c. In this way, needle body 523 has a wall section 523d having a localized wall thickness that is greater than a wall thickness of the remainder of needle body 523. A needle tip 524 is coupled to (e.g., press-fit, heat sealed), or integrally formed with (e.g., insert molded), needle body 523 at the relatively thicker wall section 523d. Like the previous example, needle tip 524 has a tapered or pointed shape sufficient to penetrate a GI lumen wall or surrounding tissue thereof.

A second alternative example of needle 122 is illustrated in FIGS. 23-24 as a needle 622. In this example, needle 622 includes two discharge openings located at or near a distal end of needle 622. Needle 622 may include more than two discharge openings, according to other embodiments. As shown in FIGS. 23-24, needle 622 includes a needle body 623 that has a needle fluid channel 623a extending longitudinally from a needle proximal end 622a to both a first discharge opening 623b1 and a second discharge opening 623b2 located opposite each other at or near a needle distal end 622b. Needle fluid channel 623a includes a first fluid channel section 623aa, a second fluid channel section 623ab, and a third fluid channel section 623ac. First fluid channel section 623aa extends from needle proximal end 622a and has a third diameter D3 that is centered along a longitudinal axis 623c of needle body 623. Second fluid channel section 623ab extends from first fluid channel section 623a to first discharge opening 623b1 at or near needle distal end 622b. Likewise, third fluid channel section 623ac extends from first fluid channel section 623a to second discharge opening 623b1 at or near needle distal end 622b opposite first discharge opening 623b1. Each of second fluid channel section 623ab and third fluid channel section 623ac has a fourth diameter D4 that is less than third diameter D3 of first fluid channel section 623aa. Further, each of second fluid channel section 623ab and third fluid channel 623ac is positioned offset from longitudinal axis 623c. In this way, needle body 623 defines a wall section 623d centered along longitudinal axis 623c. A needle tip 524 is coupled to (e.g., press-fit, heat sealed), or integrally formed with (e.g., insert molded), needle body 623 at wall section 623d. Like the other examples, needle tip 624 has a tapered or pointed shape sufficient to penetrate a GI lumen wall or surrounding tissue thereof.

Referring again to FIG. 4, valve 125 is collectively defined by a puncture member 126 and a reservoir seal 127. Puncture member 126 is movably coupled to piston 121 at valve opening 121e. In other examples, puncture member 126 may be coupled to housing 105 instead of piston 121 to interface with valve opening 121e upon sufficient axial movement of piston 121, as described herein. Puncture member 126 has a generally elongated shape with a tapered end 126a that is structured to pierce through reservoir seal 127 upon axial movement of puncture member 126 relative to piston 121. Puncture member 126 has a shaped section 126b for retaining puncture member 126 within valve opening 121e upon movement of puncture member 126 relative to piston 121. In the example shown in FIG. 4, shaped section 126b is structured as a channel extending radially inward from a side thereof which interfaces with a complementary feature (e.g., a protrusion) on piston 121 to retain puncture member 126 on piston 121 upon relative movement. This structural configuration may also help to create a substantially fluid-tight seal between puncture member 126 and piston 121 within valve opening 121e to help prevent or minimize fluid dosage form 130 from exiting through valve opening 121e during delivery of fluid dosage form 130. Puncture member 126 may be formed from or include a flexible material (e.g., silicone) to help facilitate a substantially fluid tight seal with piston 121.

Reservoir seal 127 is coupled (e.g., adhered) to piston 121 at valve opening 121e adjacent to puncture member 126 within piston interior 121c. Reservoir seal 127 is structured to contain fluid dosage form 130 within piston interior 121c. As discussed in further detail below, reservoir seal 127 is further structured to open (e.g., tear, peel, break) in response to puncture member 126 contacting reservoir seal 127. Reservoir seal 127 may be formed from or include a breakable or pierceable material such as a foil or film material (e.g., aluminum foil).

Membrane 129 is coupled to piston 121 within piston interior 121c. In the example shown, membrane 129 is coupled to an inner surface of piston 121 which defines piston interior 121c. In this example, membrane 129 may be coupled to piston 121 by heat sealing, adhering, or by other means. Membrane 129 extends circumferentially about piston 121 to enclose at least a portion or all of piston interior 121c. In this way, membrane 129, piston 121, and reservoir seal 127 cooperatively define a reservoir 129a for containing fluid dosage form 130 therein. Membrane 129 may extend axially beyond piston interior 121c to expand the volume of reservoir 129a. Depending on the size of membrane 129, reservoir 129a may define a volume for containing up to about 250 μl or more of fluid. For example, reservoir 129a may define a volume for containing from about 50 μl to about 200 μl of fluid, including from 50 μl to 250 μl of fluid, such as 50 μl, 100 μl, 150 μl, or 200 μl of fluid, or any value therebetween, or may contain 200 μl or 250 μl of fluid, or any value therebetween. As described herein, membrane 129 is structured to deform (e.g., compress, bend, flex, constrict) relative to piston 121 to expel fluid dosage form 130 from reservoir 129a upon valve 125 opening.

Additionally or alternatively, as described herein with respect to the example shown in FIG. 20, a plunger (not shown in FIG. 4, shown as plunger 931 in FIG. 20) may be movably coupled to piston 121 at piston interior 121c. The plunger may similarly extend circumferentially about piston 121 to enclose at least a portion or all of piston interior 121c. The plunger may include a perimeter seal to form a fluid-tight seal with an inner surface of piston 121 such that the plunger, piston 121, and reservoir seal 127 cooperatively define reservoir 129a for containing fluid dosage form 130 therein. The plunger may be structured to move (e.g., translate, slide) relative to piston 121 to expel fluid dosage form 130 from reservoir 129a upon valve 125 opening, as described in further detail herein.

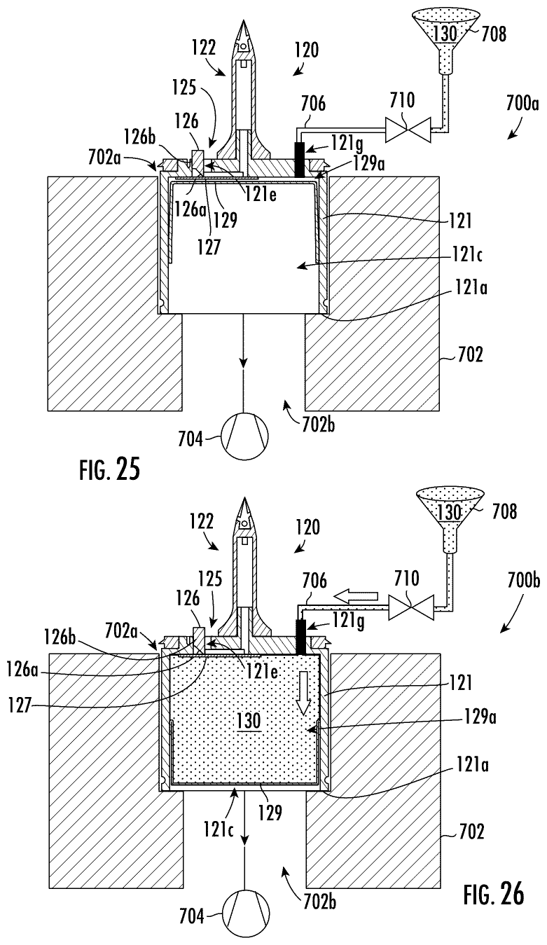

After filling (partially or fully) reservoir 129a with fluid dosage form 130, fill port 121g of piston 121 can be substantially fluidly sealed by, for example, heat sealing a portion of piston 121 that includes fill port 121g, as described in further detail with respect to FIGS. 25-27. In other examples, a separate seal (e.g., silicone or aluminum foil) may be coupled to piston 121 at fill port 121g. Additionally or alternatively, fill port 121g may include a septum (e.g., silicone septum) such that fill port 121g can self-seal after filling reservoir 129a with fluid dosage form 130. In any case, reservoir 129a may be filled with fluid dosage form 130 before piston 121 is coupled to housing 105 and balloon 102. This can, advantageously, allow for flexibility relating to aseptic assembly of device 100a.

For example, referring to FIGS. 25-27, an example method of filling first payload delivery module 120 with fluid dosage form 130 is illustrated. The disclosed method may be performed in an aseptic environment, such as in an isolator or other sufficiently sterile environment. One or more steps of the disclosed process may be performed manually or automatically using various equipment known to those skilled in the art. Although only a single payload delivery module 120 is shown in FIGS. 25-27, it should be appreciated that a plurality of payload delivery modules 120 may be filled substantially simultaneously with each other using the disclosed method. Further, the disclosed method may be used in conjunction with any of the other example payload delivery modules described herein that are structured to contain a fluid dosage form.

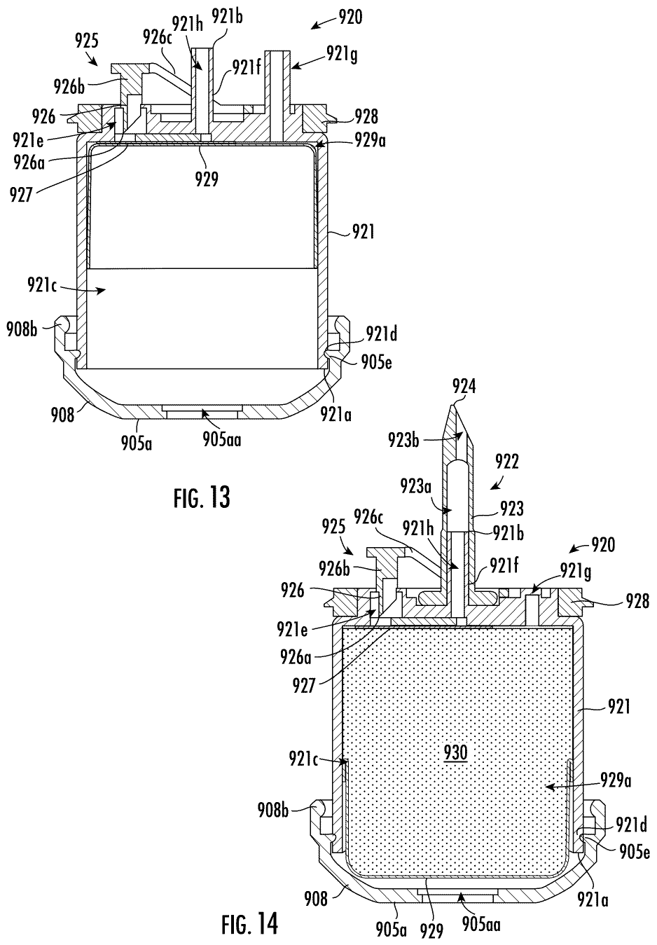

In a first filling step 700a shown in FIG. 25, an assembled first payload delivery module 120 (including piston 121, (optionally) needle 122, valve 125, and membrane 129 (and/or a plunger)) is placed in a fixture 702. In other examples, first payload delivery module 120 may include cover 108 detachably coupled together via piston release 121d and housing release 105e prior to filling (e.g., see payload delivery module 920 in FIG. 13), which cover 108 may help to define an axial stop or support feature for membrane 129 and/or plunger during the filling process. In the example shown, first payload delivery module 120 is placed in a fixture recess 702a of fixture 702. Fixture recess 702a extends longitudinally to a fixture opening 702b disposed below fixture recess 702a such that a vacuum generator 704 can be fluidly coupled to piston interior 121c. Vacuum generator 704 may be a vacuum pump or other device for generating a negative pressure.

Still referring to FIG. 25, a conduit 706 is fluidly coupled to reservoir 129a via fill port 121g. Conduit 706 may be structured as a tube, a pipe, a cannula, or other conduit suitable for delivering fluid dosage form 130 to reservoir 129a. Conduit 706 is fluidly coupled to a dosage form container 708 via a control valve 710. Dosage form container 708 includes a volume of fluid dosage form 130 disposed therein. Dosage form container 708 may be structured as a funnel, an enclosed container, or other type of container or reservoir sufficient for holding fluid dosage form 130 therein. In the example shown, the interior volume of dosage form container 708 is maintained at ambient pressure. Control valve 710 is structured to selectively control a flow of fluid dosage form 130 from dosage form container 708 to reservoir 129a via conduit 706. Control valve 710 may be structured as a pinch valve, an on/off valve, or other type of fluid control valve.

As shown in FIG. 25, control valve 710 is in an off position with substantially all of fluid dosage form 130 disposed in dosage form container 708 and in a section of conduit 706 located upstream of control valve 710. Prior to connecting conduit 706 to fill port 121g, residual air disposed between membrane 129 and piston 121 within piston interior 121c can be removed by, for example, applying a positive air pressure to an outer surface of membrane 129 through piston interior 121c from piston proximal end 121a. The residual air can then be pushed outward through fill port 121g before connecting conduit 706 thereto.

Referring now to FIG. 26, in a second filling step 700b, control valve 710 is opened to allow fluid dosage form 130 to flow from dosage form container 708 through conduit 706 to reservoir 129a via gravity. Upon opening control valve 710, a vacuum is applied to piston interior 121c via vacuum generator 704 through fixture opening 702b. As a result of the applied vacuum and the ambient pressure maintained within dosage form container 708, membrane 129 is pulled in a longitudinal direction toward piston proximal end 121a which in turn causes fluid dosage form 130 to flow into reservoir 129a (as represented by unidirectional arrows in FIG. 26) until reservoir 129a is substantially filled. In this manner, reservoir 129a can be filled with fluid dosage form 130 without using a metering pump or other complicated/costly apparatus. Further, this approach can facilitate filing a substantial amount of the volume defined by reservoir 129a with fluid dosage form 130.

In other examples, however, a fluid pump may be used to fill reservoir 129a. In these examples, the fluid pump may form part of, or otherwise be fluidly coupled with, dosage form container 708.

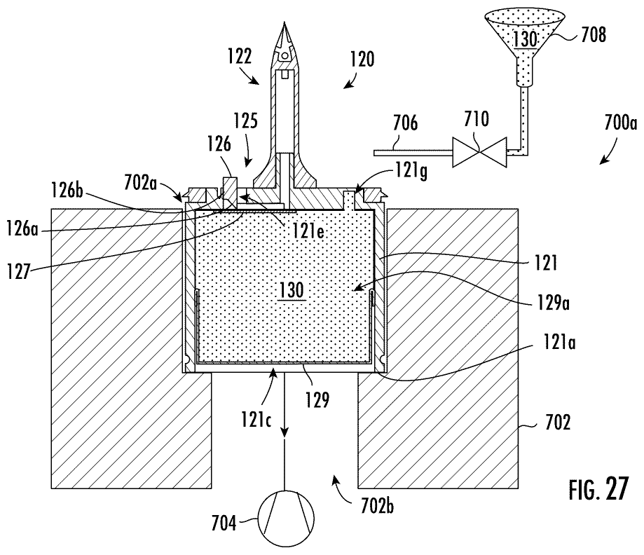

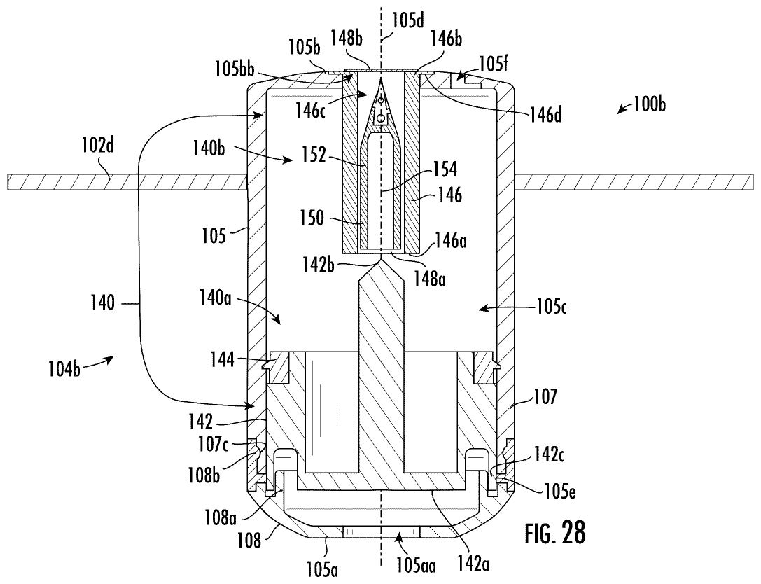

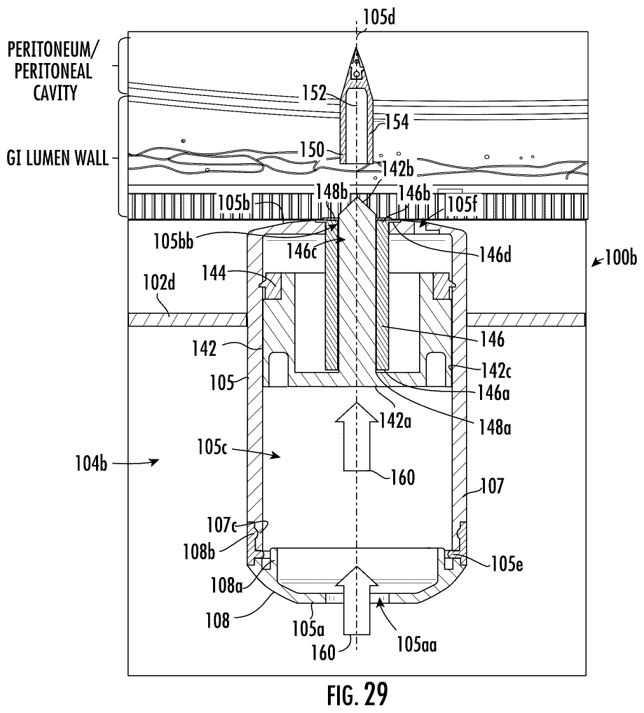

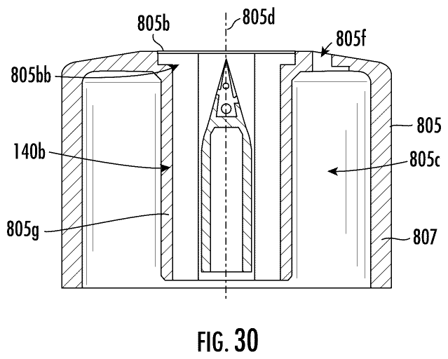

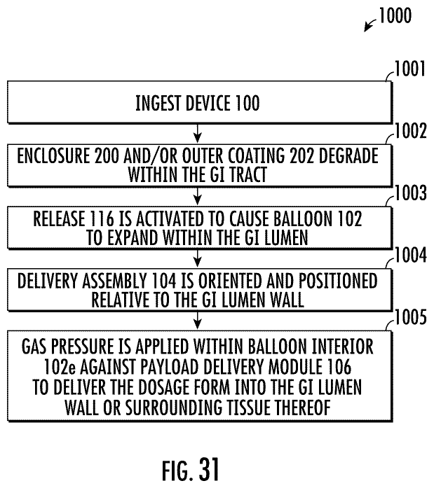

Referring now to FIG. 27, in a third filling step 700c, upon reservoir 129a being substantially filled with fluid dosage form 130, control valve 710 is closed and fill port 121g is substantially fluidly sealed to contain the volume of fluid dosage form 130 within reservoir 129a. For example, fill port 121g may be heat sealed using a heat source (e.g., a hot knife) to melt a portion of the material of piston 121 that defines fill port 121g. Upon heat sealing and sufficient cooling of fill port 121g, first payload delivery module 120 can be removed from fixture 708 for subsequent assembly with needle 122 (if not already assembled with piston 121 in the aseptic environment), housing 105, and balloon 102. Assembly with balloon 102 can, advantageously, occur outside of the aseptic environment.

As discussed below, and in the example shown in FIG. 27, membrane 129 is a flexible structure that can deform (e.g., compress, bend, flex, constrict) in response to the gas pressure generated within balloon interior 102e (from reaction of first reactant 115 and second reactant 117) reaching or exceeding a threshold value to thereby expel fluid dosage form 130 from reservoir 129a to needle 122. Prior to expelling fluid dosage form 130, membrane 129 is further structured to cause payload delivery module 120 to move axially relative to housing 105 in response to the generated gas pressure in balloon 102 being applied to an outer surface of membrane 129. Membrane 129 may have a surface profile in a relaxed state (i.e., when unfilled with fluid dosage form 130, as shown in FIG. 25) that is complementary to an inner surface profile of piston 121 which defines piston interior 121c. For example, membrane 129 may be vacuum formed using piston 121 as a mold, or by using another mold having the same/similar dimensions as piston 121, such that membrane substantially mimics the inner surface profile of piston interior 121c. In this way, membrane 129 can be sufficiently deformed to expel a substantial portion of fluid dosage form 130 from reservoir 129a. For example, device 100 may be structured such that membrane 129 is sufficiently deformed to expel about 95-100% of the volume of fluid dosage form contained in reservoir 129a, including about 95%, 96%, 97%, 98%, 99%, 100%, or any value therebetween. Further, such a complementary shape of membrane 129 may help to facilitate sufficient evacuation of reservoir 129a for subsequent filling with fluid dosage form 130 during assembly, as described above with respect to FIGS. 25-27.

Membrane 129 may be formed from or include a flexible polymeric material (e.g., polyethylene terephthalate (PET)), or other flexible material or combinations of materials having sufficiently low moisture and gas permeability properties for use with fluid dosage form 130. Membrane 129 may further include a film or coating, such as a metalized film or coating (e.g., metalized polyethylene, titanium metalized film), to help minimize or eliminate water vapor transmission through membrane 129 to fluid dosage form 130 and thereby help to prolong the shelf-life of fluid dosage form 130. The film or coating may be disposed on an outer surface of membrane 129 which is not in contact with fluid dosage form 130. Membrane 129 may be formed from a single material or a combination of materials. Further, membrane 129 may include one or more layers of a material. Membrane 129 may be a monolithic structure. In other examples, membrane 129 may be composed of multiple sections or components coupled (e.g., sealed or sewn) together. Membrane 129 may have other suitable shapes, such as cylindrical, ellipsoidal, spherical, cuboidal, or other shape.

As discussed in further detail with reference to FIG. 6, upon reaching a desired location in a GI tract of a subject, the gas that pressurizes balloon 102 can pass through first opening 105aa to apply a pressure against membrane 129 on piston 121. When the gas pressure reaches or exceeds a threshold value (e.g., a pressure associated with full or partial inflation of balloon 102 within a desired location of a GI lumen), the pressure causes payload delivery module 120 (including fluid dosage form 130) to release from housing release 105e to move axially along longitudinal axis 105d relative to housing 105 such that needle 122 penetrates the GI lumen wall. Upon sufficient axial movement of payload delivery module 120 relative to housing 105, valve 125 is structured to open (e.g., by puncture member 126 contacting housing 105 and piercing reservoir seal 127) to allow fluid dosage form 130 to flow from reservoir 129a to piston fluid channel 121h and needle 122. The gas pressure applied against membrane 129 then causes membrane 129 to deform relative to piston 121 and thereby discharge fluid dosage form 130 through piston fluid channel 121h, needle fluid channel 123a, and discharge opening 123b for delivery into the GI lumen wall or surrounding tissue of the subject.

Referring again to FIG. 4, second payload delivery module 140 includes an ejector 140a and a cartridge 140b.

Ejector 140a is structured to be releasably coupled to housing 105 within housing interior 105c. Ejector 140a is further structured to selectively move (e.g., slide) within housing interior 121c to eject solid dosage form 150 as a projectile into a GI lumen wall or surrounding tissue thereof. As shown in FIG. 4, ejector 140a includes a piston 142 and an optional piston seal 144. Piston 142 has a generally hollow cylindrical base section at a piston proximal end 142a and a generally elongated section at a piston distal end 142b. Piston proximal end 142a is generally planar and is structured to receive a force (e.g., a gas pressure) through housing proximal end 105a to cause axial movement of ejector 140a relative to housing 105, as discussed in further detail herein. Piston distal end 142b is generally elongated with a tapered end for ejecting solid dosage form 150 from cartridge 140b into a GI lumen wall or surrounding tissue thereof.

Optional piston seal 144 is coupled to an outer periphery of piston 142. Piston seal 144 is structured to create a substantially fluid tight seal between piston 142 and housing 105 to, for example, substantially prevent gas within balloon interior 102e from passing between piston 142 and housing 105. Piston seal 144 is further structured to allow relative (sliding) movement between piston 142 and housing 105.

Like piston 121, a lateral wall (e.g., an outer circumferential surface) of the base section of piston 142 includes a piston release 142c (e.g., release feature, trigger) that is complementary to housing release 105e for releasably coupling ejector 140a to housing 105. In the example shown, piston release 142c is structured as a channel that is complementary to the protrusion of housing release 105e to define a press-fit arrangement, although other structural configurations are contemplated (e.g., a protrusion, a frangible connection) depending on the structure of housing release 105e. As described below, piston 142 is structured to release from housing release 105e in response to a force applied to piston 142 through first opening 105aa reaching or exceeding a threshold value to cause piston 142 to move axially within housing interior 105c toward the GI lumen wall. For example, the force may be a gas pressure generated within balloon interior 102e by a gas generating mechanism (e.g., mixing of first reactant 115 with second reactant 117). In this example, the threshold gas pressure may be in a range of about 10-40 psi, including from about 20-40 psi, such as about 20 psi, 25 psi, 30 psi, 35 psi, 40 psi, or any value therebetween. In other examples, the force may be a mechanical or a fluidic force generated by a separate mechanism (e.g., a spring, a hydrogel) operatively coupled to piston 142.

Still referring to FIG. 4, cartridge 140b includes a container 146 and a solid dosage form 150 disposed therein. Cartridge 140b is structured to be coupled to housing 105 at housing distal end 105b via container 146. Specifically, in the example shown, container 146 has a generally hollow cylindrical shape that has a container proximal end 146a and a container distal end 146b. Container distal end 146b includes a flange 146d for coupling cartridge 140b to housing 105 at housing distal end 105b via a press-fit arrangement. In other examples, container 140 and housing 105 may include a different type of attachment interface, such as a snap-fit interface, a bayonet attachment, or other interface.

In yet another example shown in FIG. 30, which is discussed in further detail below, housing 105 may define a sleeve or similar structure at housing distal end 105b to receive container 146 therein.

Still referring to FIG. 4, a cavity 146c (e.g., interior) extends from container proximal end 146a to container distal end 146b. Cavity 146c is structured to receive solid dosage form 150 therein. Container proximal end 146a includes an opening to cavity 146c but includes a first seal 148a coupled thereto (e.g., adhered). First seal 148a is structured to be pierced or punctured by piston distal end 142b to eject solid dosage form 150 as a projectile from container 146. Similarly, container distal end 146b includes an opening to cavity 146c but includes a second seal 148b coupled thereto (e.g., adhered). In this way, first seal 148a and second seal 148b can substantially seal cavity 146c to provide a substantially sterile environment to preserve solid dosage form 150 for subsequent delivery into a GI lumen wall or surrounding tissue thereof. Similar to first seal 148a, second seal 148b is structured to be pierced or punctured by solid dosage form 150 to allow ejection of solid dosage form 150 from container 146 into a GI lumen wall or surrounding tissue thereof. First seal 148a and second seal 148b may be formed from or include a penetrable or breakable material, such as a foil (e.g., aluminum foil), film, or other material or combinations of materials.

Solid dosage form 150 is shown to include a needle structure 152 and a composition 154 disposed therein. Needle structure 152 has a generally elongated shape with a tapered or pointed end sufficient to pierce through second seal 148b and penetrate the GI lumen wall or surrounding tissue. Needle structure 152 is further structured to receive a force from ejector 140a at an end opposite the tapered end to propel or eject needle structure 152 as a projectile from container 146. Needle structure 152 may be formed from or include a biodegradable material (e.g., PEO, PE) to degrade in situ upon delivery into a GI lumen wall or surrounding tissue to thereby expose composition 154 for systemic uptake of one or more therapeutic agents contained therein.

Composition 154 is shown as a solid cylindrical member disposed within an interior of needle structure 152. Composition 154 may include one or more therapeutic agents as described herein along with one or more additional components, such as a binder, a preservative, a disintegrant, a lubricant, or other component. As explained above, composition 154 may be a compressed tablet formed from a lyophilized powder that includes one or more biologically active therapeutic agents. Composition 154 may have a weight of about 1-3 milligrams (mg) and may contain a dosage of about 1-10 mg of one or more therapeutic agents. In the example shown, composition 154 is structured to degrade in situ upon exposure to the GI lumen or surrounding tissue environment (e.g., upon sufficient degradation of needle structure 152) to release one or more therapeutic agents contained therein for systemic uptake in a subject.

In other examples, composition 154 may be in a different constitution, such as a gel, a liquid, a suspension, a powder, or other constitution for being contained in the cavity of needle structure 152.

In still other examples, composition 154 itself may be formed (e.g., compressed in a mold) into the shape of a needle or other tapered/pointed structure for penetrating a GI lumen wall or surrounding tissue without a separate needle structure 152.

FIG. 5 is a partial cross-sectional view of an ingestible device 100a (an embodiment of ingestible device 100) including first modular delivery assembly 104a coupled to balloon 102. Device 100a is shown in a first state before inflation of balloon 102 within a desired location of a GI tract of a subject for delivery of fluid dosage form 130. As shown in FIG. 5, first payload delivery module 120 is releasably coupled to, and slidably disposed in, housing 105. Specifically, piston 121 is releasably coupled to housing 105 via a press-fit arrangement between piston release 121d and housing release 105e. First payload delivery module 120 is positioned within housing interior 105c with needle 122 located adjacent second opening 105bb. A needle seal 110 is coupled (e.g., adhered) to housing 105 at housing distal end 105b to substantially seal second opening 105bb and thereby help to maintain sterility of needle 122 within housing interior 105c. Needle seal 110 may be formed from a breakable material, such as a foil material (e.g., aluminum foil). Needle 122 is structured to pierce through needle seal 110 to penetrate a GI lumen wall or surrounding tissue thereof.

An optional vent container 132 is shown coupled to housing 105 at or near housing distal end 105b. Vent container 132 is coupled (e.g., heat sealed, adhered) to a periphery of housing 105 and to an upper surface of housing distal end 105b adjacent to second opening 105bb to define a generally ring-shaped (e.g., donut-shaped) structure having an interior volume 132a. Interior volume 132a is in fluid communication with housing interior 105c via vent opening 105f to capture pressurized residual air contained in housing interior 105c above piston 121 after actuation (i.e., axial movement) of piston 121. Interior volume 132a may be larger than the volume of housing interior 105c to ensure that substantially all of the residual air contained in housing interior 105c is captured within vent container 132 without causing significant or any expansion of vent container 132. In this way, vent container 132 may help to prevent the pressurized residual air in housing interior 105c that exits vent opening 105f from interfering with needle 122 upon deployment from housing 105. Vent container 132 may be formed from or include a flexible material or a combination of materials, such as a flexible polymeric material (e.g., hydroxypropyl methylcellulose (HPMC), PE). Vent container 132 may optionally be used in combination with any of the example devices and assemblies described herein.

FIG. 6 illustrates ingestible device 100a in a second state after device 100a has reached a desired location in the GI tract (e.g., the stomach, small intestine, large intestine) of a subject for delivering fluid dosage form 130. In this example, device 100a is shown in the small intestine. As shown in FIG. 6, balloon 102 has been inflated by a gas generated within balloon interior 102e (as discussed above with reference to FIGS. 1-2) such that elongated section 102d is substantially aligned with a surface of the GI lumen wall and housing distal end 105b of housing 105 is oriented and positioned relative to (e.g., proximate to or in contact with) the surface of the lumen wall. The generated gas within balloon interior 102e applies a pressure (indicated by unidirectional arrows 160) against an outer surface of membrane 129 through first opening 105aa of housing 105. When the gas pressure reaches or exceeds a threshold value (e.g., a pressure associated with a fully or partially inflated state of balloon 102, such as a pressure of about 10-40 psi), piston release 121d overcomes the press-fit interface with housing release 105e. As a result, the entire payload delivery module 120 including fluid dosage form 130 moves axially relative to housing 105 along longitudinal axis 105d toward the lumen wall such that needle tip 124 is advanced through needle seal 110 to penetrate the lumen wall or surrounding tissue thereof. During at least a portion of the axial travel of payload delivery module 120, the axial orientation of piston 121 may be assisted by inner wall 108a that projects from cover 108.

The axial movement of payload delivery module 120 may generate a back pressure from residual air contained within housing interior 105c above piston 121. Accordingly, vent opening 105f may allow for venting of the resulting back pressure generated within housing interior 105c, as well as gas contained within balloon interior 102e after delivery of fluid dosage form 130, to help facilitate axial movement of piston 121 relative to housing 105. Optional vent container 132 may capture a substantial portion of, or all of, the residual air from housing interior 105c to help minimize potential interference between the air exiting vent opening 105f with needle 122 when needle 122 is advanced through second opening 105bb.

In the example shown in FIG. 6, needle 122 is advanced through the lumen wall and into the peritoneal cavity of the subject. In other examples, needle 122 has a different length or is advanced to a different penetration depth, such as into a layer of the GI lumen wall (e.g., mucosa, submucosa, muscularis, serosa) for discharging fluid dosage form 130 therein. Upon sufficient axial travel of payload delivery module 120 relative to housing 105 (e.g., such that needle 122 is advanced to a desired penetration depth), puncture member 126 engages an inner surface of housing 105 near distal end 105b to cause puncture member 126 to move relative to piston 121 within valve opening 121e and pierce reservoir seal 127. Upon puncture member 126 piercing reservoir seal 127, the gas pressure applied against membrane 129 (represented by unidirectional arrows 160) causes membrane 129 to deform inward relative to piston 121 toward needle 122 to expel fluid dosage form 130 from reservoir 129a through reservoir seal 127 to needle 122. As a result, fluid dosage form 130 is directed through piston fluid channel 121h, needle fluid channel 123a, and needle discharge opening 123b into the peritoneal cavity. In this manner, one or more therapeutic agents contained in fluid dosage form 130 can be delivered to the subject for systemic uptake.

As described above, fluid dosage form 130 remains in reservoir 129a until puncture member 126 pierces reservoir seal 127 while the gas pressure in balloon 102 is applied against membrane 129. In this manner, device 100a can allow for sequential timing between penetrating the GI lumen wall and discharging fluid dosage form 130 to substantially avoid discharging fluid dosage form 130 into the lumen environment and ensure delivery of fluid dosage form 130 into the GI lumen wall or surrounding tissue for systemic uptake.

Upon completing delivery of fluid dosage form 130, one or more components of device 100a (e.g., balloon 102, needle 122) can subsequently degrade within the GI lumen wall or surrounding tissue, or other area within the GI tract. For example, one or more components of device 100a may be formed from or include one or more biodegradable materials to facilitate degradation of such components in situ before, during, or after delivery of fluid dosage form 130. Examples of biodegradable materials that may be suitable for use with various components of device 100a include, for example, hydroxypropyl methylcellulose (HPMC), polyvinyl acetate (PVA), PEO, PEG, PGLA, lactide, glycolide, lactic acid, glycolic acid, par-dioxanone, trimethylene carbonate, caprolactone, and mixtures and copolymers thereof.

In one or more embodiments, and particularly in embodiments where needle 122 is substantially non-degradable or includes substantially non-degradable portions, device 100a can be structured to retract needle 122 from the GI lumen wall into housing interior 105c upon completing delivery of fluid dosage form 130 (e.g., using a biasing component such as a spring coupled to needle 122). In either of these embodiments, vent openings in housing 105 and/or deflation valve 112 can release a substantial amount of the gas contained within balloon interior 102a to allow for substantial deflation of balloon 102 and subsequent traversal of device 100a through the remainder of the GI tract to exit the anus of the subject.

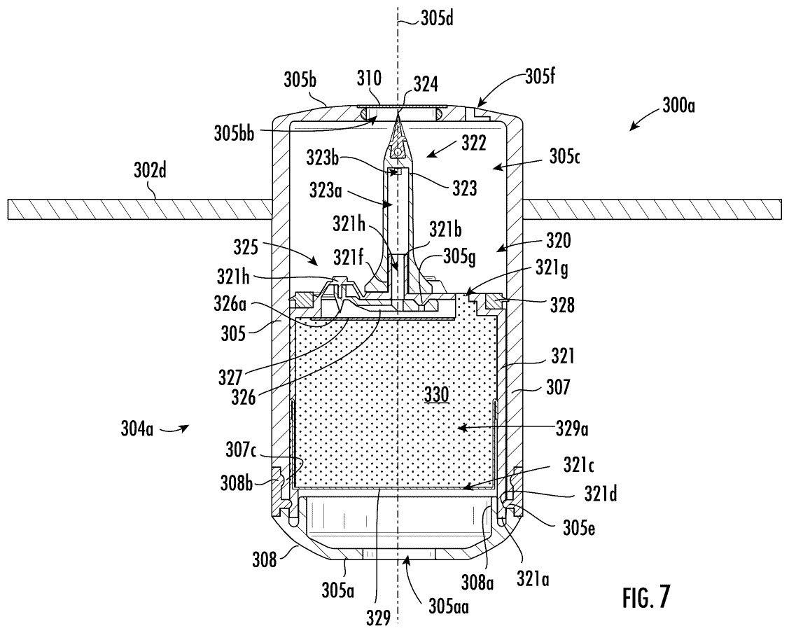

FIG. 7 is a partial cross-sectional view of an ingestible device 300a (an embodiment of ingestible device 100) including another version of payload delivery module 120 (shown as payload delivery module 320) with a different valve arrangement. Aside from the valve arrangement, the structure of payload delivery module 320 is the same as payload delivery module 120. Accordingly, for the sake of efficiency, like reference numerals refer to like components between examples, but are increased by an order of two (e.g., membrane 329 is equivalent to membrane 129, etc.). In this example, payload delivery module 320 does not include a valve opening on piston 321. Rather, payload delivery module 320 includes a valve 325 that is collectively defined by a base 321h, a puncture member 326, and a reservoir seal 327.

Still referring to FIG. 7, base 321h has a dome shape and is integrally formed with piston 321. In other examples, base 321h may be a separate component that is coupled to piston 321. Further, base 321h may have other shapes besides a dome, such as a trapezoid or other shape. As described below, base 321h projects from an outer surface of piston 321 and is structured to deform (e.g., deflect, bend, turned inside out) inward toward reservoir seal 327 upon contacting an inner surface of housing 305 in response to axial movement of piston 321 relative to housing 305.

Puncture member 326 is coupled (e.g., press-fit) or positioned adjacent to base 321h. Puncture member 326 is also pivotally coupled to a portion 305g of housing 305 adjacent to housing distal end 321b within piston interior 321c. In the example shown, portion 305g is structured as a frusto-conical protrusion which defines a fulcrum to pivotally couple (e.g., press-fit) puncture member 326 thereto. In particular, puncture member 326 is cantilevered from portion 305g to allow for pivotal movement of puncture member 326 with base 321h upon base 321h contacting housing 305. Puncture member 326 further includes a tip 326a that is positioned adjacent to reservoir seal 327 and is structured to pierce reservoir seal 327 upon deformation of base 321h.

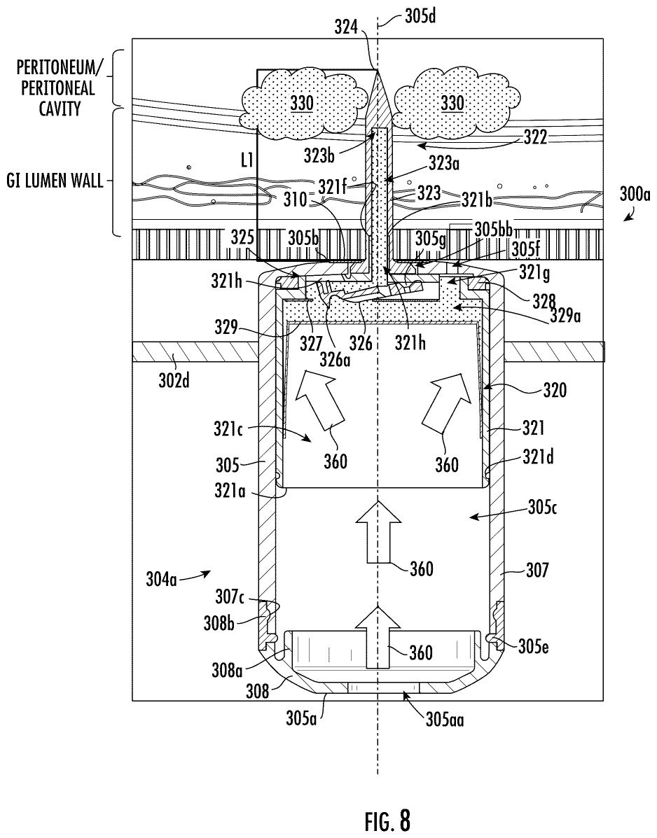

FIG. 8 illustrates ingestible device 300a in a second state after device 300a has reached a desired location in the GI tract (e.g., the stomach, small intestine, large intestine) of a subject for delivering fluid dosage form 330. In this example, device 300a is shown in the small intestine. As shown in FIG. 8, upon sufficient axial travel of payload delivery module 320 relative to housing 305 (e.g., such that needle 322 is advanced to a desired penetration depth), base 321h engages an inner surface of housing 305 near housing distal end 305b to cause base 321h to deform inward toward reservoir seal 327. As a result, tip 326a of puncture member 326 moves in response to deformation of base 321h to pierce reservoir seal 327. Upon tip 326a piercing reservoir seal 327, the gas pressure (represented by unidirectional arrows 360) applied against membrane 329 causes membrane 129 to deform inward relative to piston 321 toward needle 322 to expel fluid dosage form 330 from reservoir 329a through reservoir seal 327 to needle 322. As a result, fluid dosage form 330 is directed through piston fluid channel 321h, needle fluid channel 323a, and needle discharge opening 323b into the peritoneal cavity. In this manner, one or more therapeutic agents contained in fluid dosage form 330 can be delivered to the subject for systemic uptake.

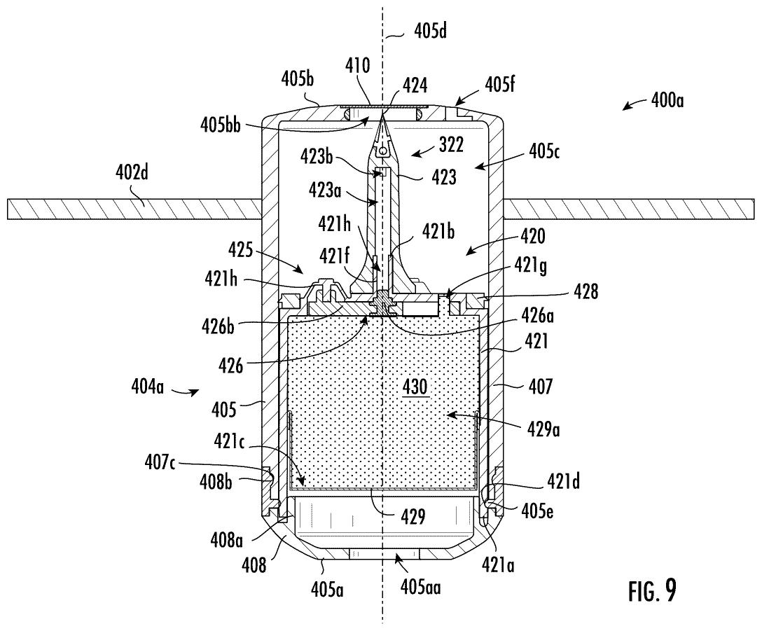

FIG. 9 is a partial cross-sectional view of an ingestible device 400a (an embodiment of ingestible device 100) including yet another version of payload delivery module 120 (shown as payload delivery module 420) with another valve arrangement. Aside from the valve arrangement, the structure of payload delivery module 420 is the same as payload delivery module 120. Accordingly, for the sake of efficiency, like reference numerals refer to like components between examples, but are increased by an order of three (e.g., membrane 429 is equivalent to membrane 129, etc.). In this example, payload delivery module 420 does not include a valve opening or reservoir seal on piston 421. Rather, payload delivery module 420 includes a valve 425 that is collectively defined by a base 421h and a plug 426.

Still referring to FIG. 9, base 421h has a dome shape and is integrally formed with piston 421, like the structure of base 321h described above with reference to FIGS. 7-8. In other examples, base 421h may be a separate component that is coupled to piston 421. Further, base 421h may have others shapes besides a dome, such as a trapezoid or other shape. As described below, base 421h projects outwardly from an outer surface of piston 421 and is structured to deform (e.g., deflect, bend, turned inside out) inward toward reservoir 429a upon contacting an inner surface of housing 405 in response to axial movement of piston 421 relative to housing 405. In various examples, valve 425 may include one or more bases 421h positioned along an upper portion of piston 421.

Plug 426 includes a sealing portion 426a and an attachment portion 426b. Sealing portion 426a may be coupled (e.g., adhered, fastened) to, or integrally formed (e.g., insert molded) with, attachment portion 426b. Sealing portion 426a is releasably coupled to piston 421 at piston fluid channel 421h. In the example shown, sealing portion 426a is press-fit into piston fluid channel 421h and substantially fluidly seals reservoir 429a from piston fluid channel 421h to substantially prevent fluid dosage form 430 from entering piston fluid channel 421h. For example, sealing portion 426a may be formed from, or include, a material structured to create a fluidic seal with piston 421, such as a flexible polymeric material (e.g., silicone) or other material or combinations of materials. Attachment portion 426b is coupled to, or otherwise in contact with, base 421h to allow for a transfer of force from base 321h to plug 426.

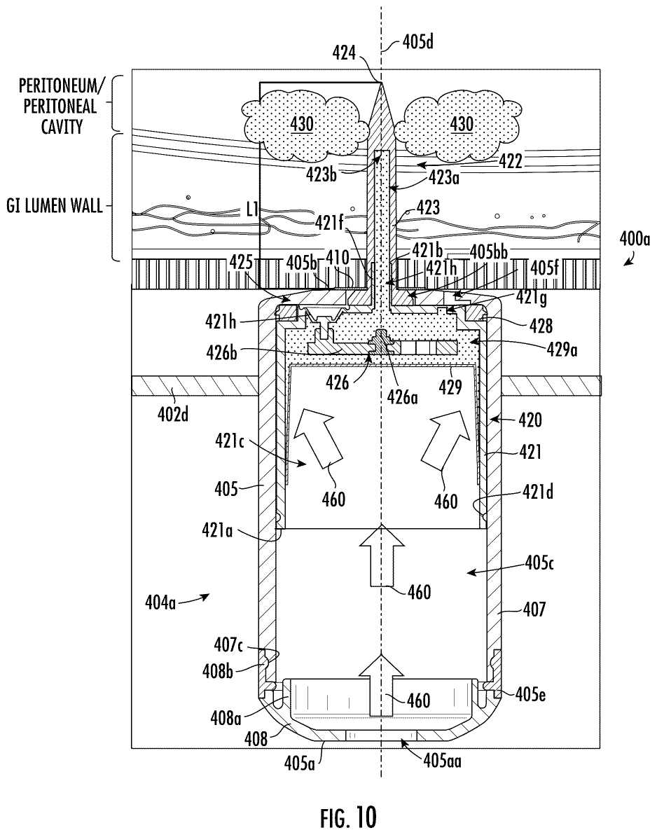

FIG. 10 illustrates ingestible device 400a in a second state after device 400a has reached a desired location in the GI tract (e.g., the stomach, small intestine, large intestine) of a subject for delivering fluid dosage form 430. In this example, device 400a is shown in the small intestine. As shown in FIG. 10, upon sufficient axial travel of payload delivery module 420 relative to housing 405 along longitudinal axis 405d (e.g., such that needle 422 is advanced to a desired penetration depth), base 421h engages an inner surface of housing 405 near distal end 405b to cause base 421h to deform inward toward reservoir 429a. In this example, base 421h is deformed to an inverted position within piston interior 421c. The deformation of base 421h causes attachment portion 426b to move axially toward reservoir 429a to thereby cause sealing portion 426a to release from needle port 421f and piston fluid channel 421h. Upon release of sealing portion 426a from piston fluid channel 421h, the gas pressure (represented by unidirectional arrows 460) applied against membrane 429 causes membrane 429 to deform inward relative to piston 421 toward needle 422 to expel fluid dosage form 430 from reservoir 429a to needle 422. As a result, fluid dosage form 430 is directed through piston fluid channel 421h, needle fluid channel 423a, and needle discharge opening 423b into the peritoneal cavity. In this manner, one or more therapeutic agents contained in fluid dosage form 430 can be delivered into the subject's blood stream for systemic uptake.

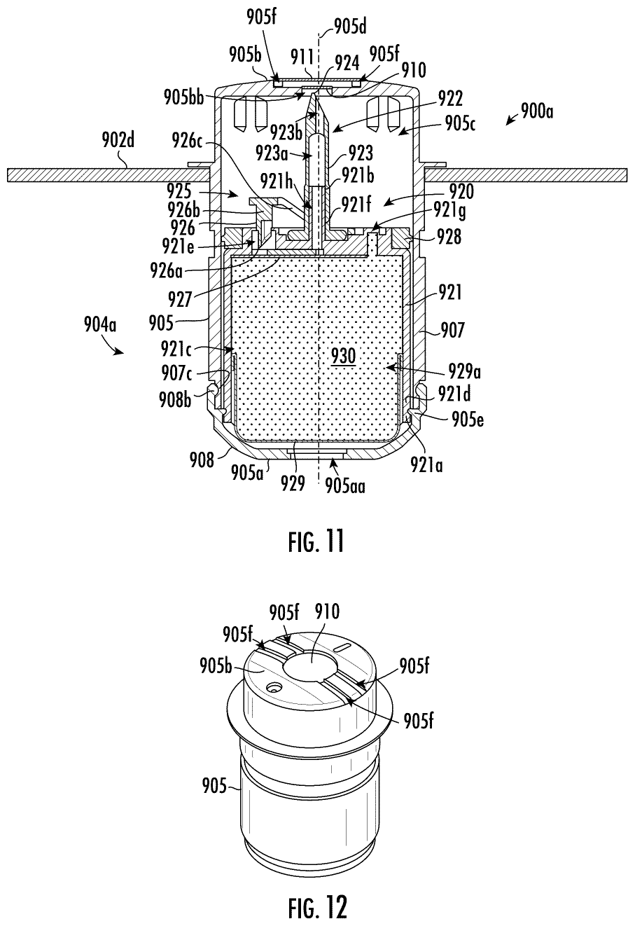

FIG. 11 is a partial cross-sectional view of an ingestible device 900a (an embodiment of ingestible device 100) including yet another version of payload delivery module 120 (shown as payload delivery module 920) shown with a different valve arrangement and vent path. Payload delivery module 920 and housing 905 are otherwise the same as payload delivery module 120 and housing 105, respectively. Accordingly, for the sake of efficiency, like reference numerals refer to like components between examples, but are increased by an order of eight (e.g., membrane 929 is equivalent to membrane 129, etc.).

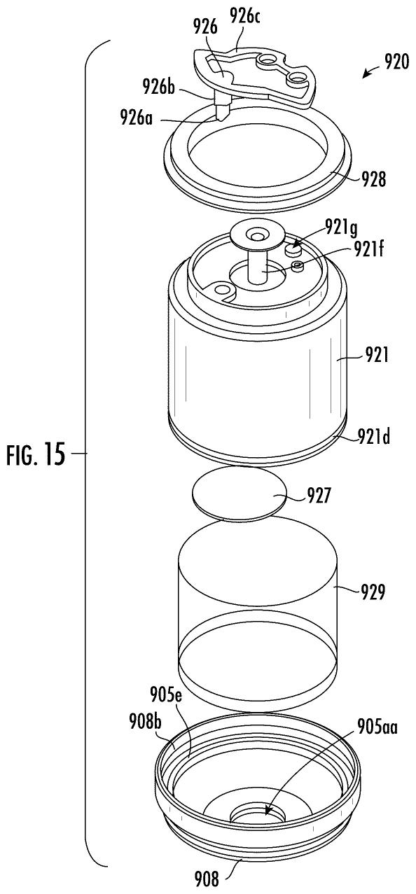

As shown in FIGS. 11 and 13-15, payload delivery module 920 includes a valve 925 that is collectively defined by a puncture member 926 and a reservoir seal 927. In this example, puncture member 926 is a unitary structure including a puncture member tip 926a, a puncture member body 926b, and a puncture member arm 926c. Puncture member 926 is coupled (e.g., heat staked, press-fit) to piston 921 via puncture member arm 926c such that puncture member body 926b and tip 926a are positioned adjacent to valve opening 921e above reservoir seal 927. In other examples, puncture member 926 (e.g., tip 926a) is coupled to, or forms part of, housing 905. As described herein, puncture member body 926b and tip 926a are structured to move via deformation of puncture member arm 926c in response to sufficient axial movement of payload delivery module 920 relative to housing 905. Puncture member tip 926a is structured to pierce reservoir seal 927 in response to puncture member base 926b engaging housing 905 upon sufficient axial movement of payload delivery module 920. Puncture member base 926b is further structured to create a substantially fluid-tight seal with piston 921 via an interference fit in valve opening 921e upon puncture member tip 926a piercing reservoir seal 927. Puncture member base 926b may include a separate seal (e.g., silicone) to help create a substantially fluid-tight seal with piston 921. Puncture member 926 may be formed from or include a polymeric material, such as polypropylene, polyethylene, or other material or combinations of materials (e.g., surgical grade steel).

FIG. 13 illustrates payload delivery module 920 prior to filling reservoir 929a with fluid dosage form 930 via fill port 921g. In this example, payload delivery module 920 is shown without needle 922 coupled to needle port 921f. Further, cover 908 is shown releasably coupled to piston 921 via housing release feature 905e and piston release feature 921d, such that cover 908 can function as a stop feature for membrane 929 during the filling process. It should be appreciated that payload delivery module 920 may be filled with fluid dosage form 930 in the same manner as payload delivery module 120 as described herein with respect to FIGS. 25-27.

FIG. 14 illustrates payload delivery module 920 after filling reservoir 929a with fluid dosage form 930. Payload delivery module 920 is shown with needle 922 coupled to needle port 921f and with fill port 921g substantially fluidly sealed. Payload delivery module 920 may be subsequently coupled with body 907 of housing 905 via cover 908 to define delivery assembly 904a.

Referring to FIGS. 11-12, a vent seal 911 (not shown in FIG. 12 for ease of view) is coupled to housing 905 above needle seal 910. Housing 905 further includes one or more vent channels 905f that are oriented laterally relative to longitudinal axis 905d. Vent channels 905f extend laterally from second opening 905bb to an outermost side periphery of housing 905 to provide a vent path from housing interior 905c to the GI lumen environment. The lateral orientation of vent channels 905f can, advantageously, help to prevent back pressure from pushing (e.g., stretching) the GI lumen wall away from device 900a upon needle 922 piercing the GI lumen wall, since gas is vented laterally away from longitudinal axis 905d. As shown in FIGS. 11 and 12, vent channels 905f are disposed between, and are partially defined by, vent seal 911 (not shown in FIG. 12) and one or more slots disposed in housing 905. Needle 922 is structured to penetrate through both needle seal 910 and a portion of vent seal 911. However, vent seal 911 remains intact over vent channels 905f to allow for lateral venting of gas from housing 905 and balloon interior 902e to the GI lumen environment upon piercing of needle seal 910.

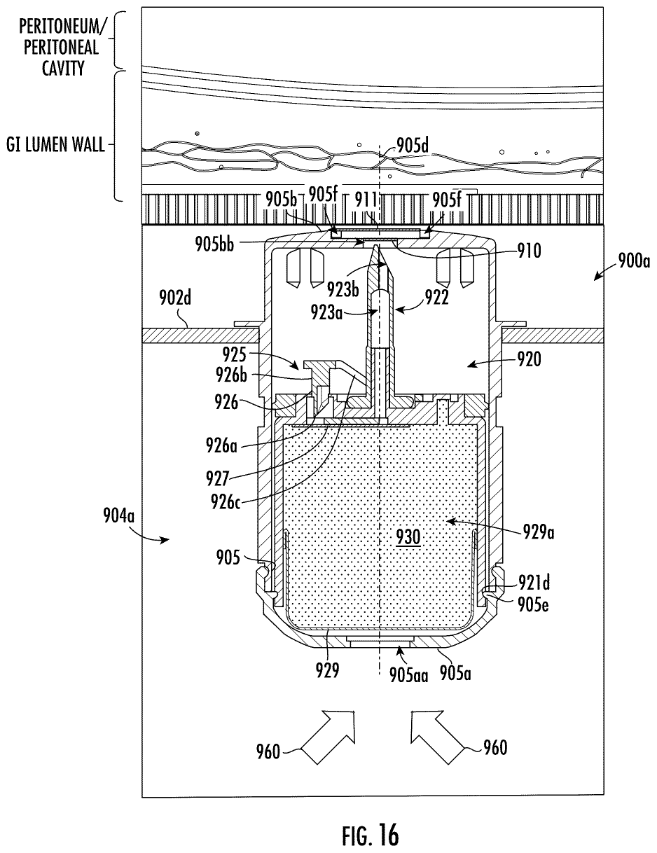

Referring to FIGS. 16-19, an example actuation sequence of ingestible device 900a in a GI tract of a subject is shown after ingestion of device 900a. FIG. 16 illustrates device 900a in a second state after device 900a has reached a desired location in the GI tract (e.g., the stomach, small intestine, large intestine) of the subject for delivering fluid dosage form 930. In this example, device 900a is shown in the small intestine. As shown in FIG. 16, and as discussed above with reference to FIGS. 1-2, balloon 902 (not shown) has been inflated by a gas (represented by directional arrows 960) generated within balloon interior 902e (not shown) such that elongated section 902d is substantially aligned with a surface of the GI lumen wall and housing distal end 905b is oriented and positioned relative to (e.g., proximate to or in contact with) the surface of the GI lumen wall.

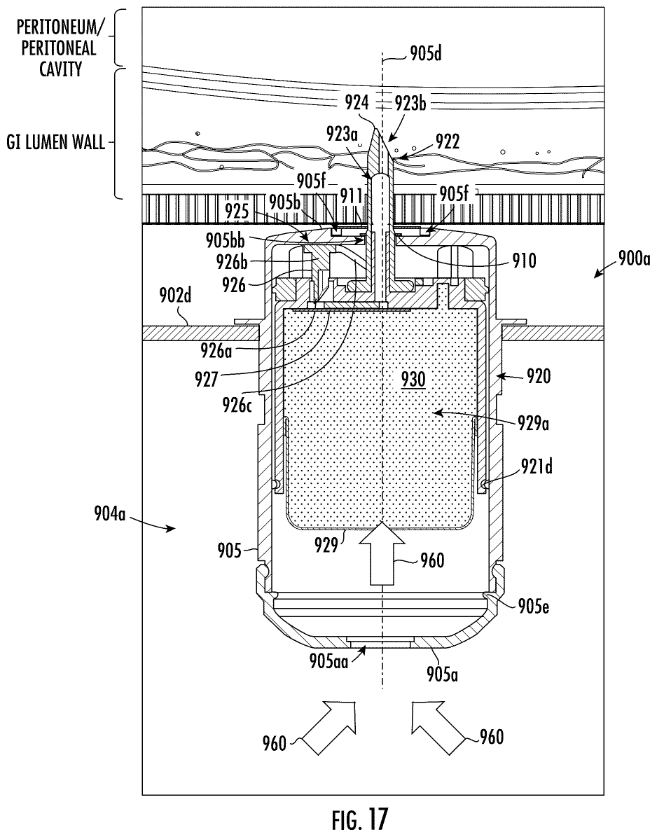

Referring to FIG. 17, when the gas pressure reaches or exceeds a threshold value (e.g., a pressure associated with a fully or partially inflated state of balloon 902 (not shown), such as a pressure of about 10-40 psi), piston release 921d overcomes the press-fit interface with housing release 905e. As a result, the entire payload delivery module 920 including fluid dosage form 930 moves axially relative to housing 905 along longitudinal axis 905d toward the GI lumen wall such that needle tip 924 is advanced through needle seal 910 and a portion of vent seal 911 to penetrate the GI lumen wall or surrounding tissue thereof.

The axial movement of payload delivery module 920 may generate a back pressure from residual air contained within housing interior 905c above piston 921. Accordingly, vent channels 905f can allow for venting of the resulting back pressure generated within housing interior 905c, as well as gas contained within balloon interior 902e (not shown) after delivery of fluid dosage form 930, laterally away from housing 905 upon piercing of needle seal 910. Gas contained in housing interior 905c can travel longitudinally through second opening 905bb then laterally through vent channels 905f and into the GI lumen environment such that housing distal end 905b remains positioned relative to the GI lumen wall.

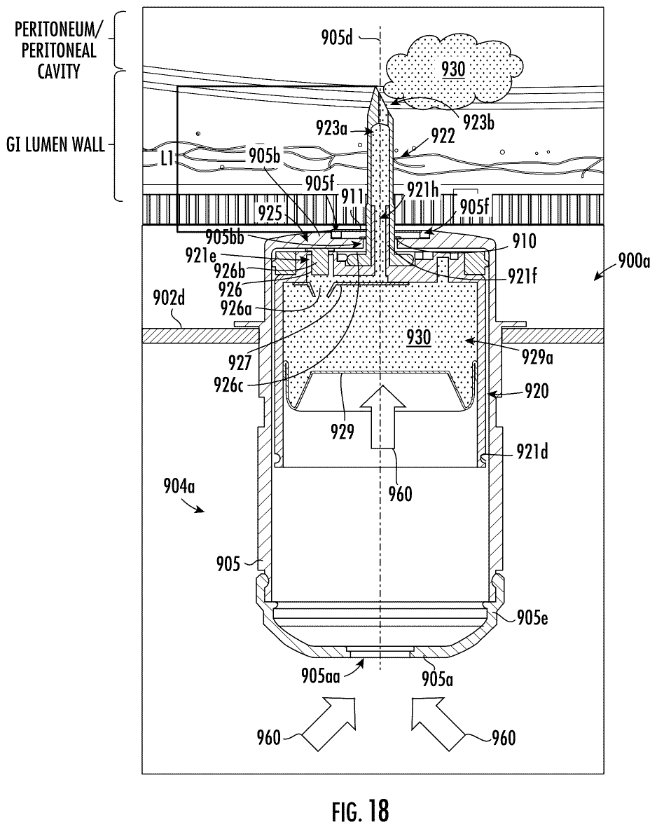

Referring to FIG. 18, upon sufficient axial travel of payload delivery module 920 relative to housing 905 (e.g., such that needle 922 is advanced to a desired penetration depth; in this example, the peritoneal cavity), puncture member body 926b engages an inner surface of housing 905 near housing distal end 905b. This, in turn, causes puncture member 926 to deform (e.g., bend, pivot) relative to piston 921 via puncture member arm 926c such that puncture member tip 926a is advanced through valve opening 921e to pierce reservoir seal 927. Sufficient relative movement of puncture member 926 causes puncture member body 926b to create a substantially fluid tight seal with piston 921 in valve opening 921e via an interference fit.

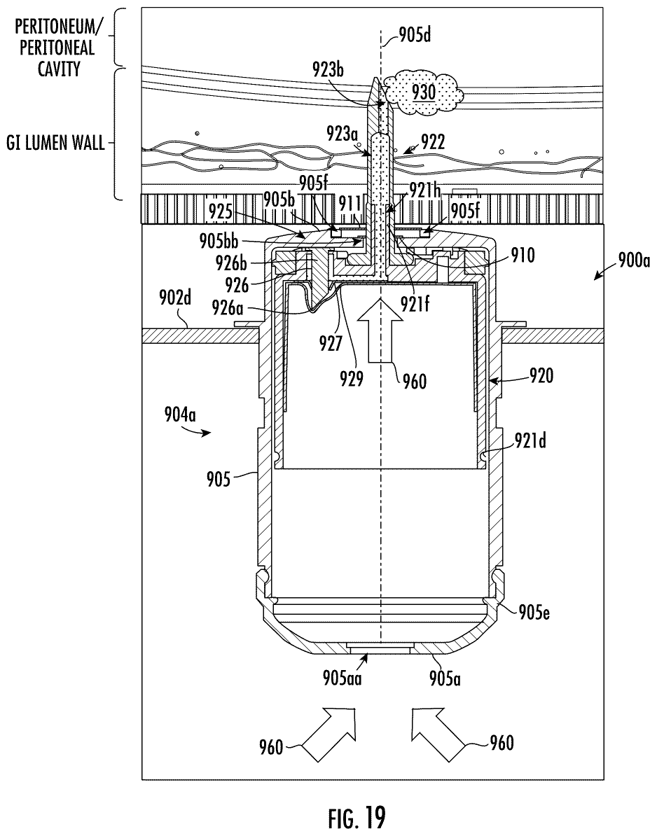

Referring to FIGS. 18-19, upon puncture member 926 piercing reservoir seal 927, the gas pressure applied against membrane 929 (represented by directional arrows 960) causes membrane 929 to deform inward relative to piston 921 toward needle 922 to thereby expel fluid dosage form 930 from reservoir 929a to needle 922 through the pierced opening in reservoir seal 927. As a result, fluid dosage form 930 is directed through piston fluid channel 921h, needle fluid channel 923a, and needle discharge opening 923b into the peritoneal cavity until reservoir 929a is substantially evacuated of fluid dosage form 930. In this manner, one or more therapeutic agents contained in fluid dosage form 930 can be delivered to the subject for systemic uptake.

As described above, fluid dosage form 930 remains in reservoir 929a until puncture member 926 pierces reservoir seal 927 while the gas pressure in balloon 902 (not shown) is applied against membrane 929. In this manner, device 900a can allow for sequential timing between penetrating the GI lumen wall or surrounding tissue and discharging fluid dosage form 930 to substantially avoid discharging fluid dosage form 930 into the lumen environment and ensure delivery of fluid dosage form 930 into the GI lumen wall or surrounding tissue for systemic uptake.

FIG. 20 illustrates an ingestible device 900a′ (another embodiment of ingestible device 100) including a different version of payload delivery module 920 (shown as payload delivery module 920a) shown with a plunger 931 instead of membrane 929. Payload delivery module 920a is otherwise the same as payload delivery module 920. Accordingly, for the sake of efficiency, like reference numerals refer to like components between examples.

As shown in FIG. 20, plunger 931 is movably (e.g., slidably) coupled to piston 921 in piston interior 921c to define a reservoir 931a for containing fluid dosage form 930. In this example, plunger 931 includes a seal 932 coupled to an outer periphery thereof for sealingly engaging piston 921 to substantially contain fluid dosage form 930 within reservoir 931a. Plunger 931 is shown partially disposed in piston interior 921c to allow for relative sliding movement between plunger 931 and piston 921. Reservoir 931a may define a volume for containing about 50-250 μl of fluid therein, including about 50 μl, 100 μl, 150 μl, 200 μl, 250 μl, or any value therebetween.

Payload delivery module 920a can be filled with fluid dosage form 930 in the same manner as payload delivery module 920 described herein. For example, a vacuum can be pulled on plunger 931 through first opening 905aa to cause fluid dosage form 930 to fill reservoir 931a until plunger 931 contacts cover 908.

Device 900a can have the same actuation sequence as device 900 described herein. For example, upon puncture member 926 piercing reservoir seal 927, the gas pressure (not shown) applied against plunger 931 through opening 905aa would cause plunger 931 to move relative to piston 921 toward needle 922 to thereby expel fluid dosage form 930 from reservoir 931a to needle 922 through the pierced opening in reservoir seal 927. As a result, fluid dosage form 930 would be directed through piston fluid channel 921h, needle fluid channel 923a, and needle discharge opening 923b into the GI lumen wall or surrounding tissue thereof until reservoir 931a is substantially evacuated of fluid dosage form 930.

FIG. 24 is a partial cross-sectional view of an ingestible device 100b (another embodiment of ingestible device 100) including second modular delivery assembly 104b coupled to balloon 102. Device 100b is shown in a first state before inflation of balloon 102 within a desired location of a GI tract of a subject for delivery of solid dosage form 150. As shown in FIG. 24, second payload delivery module 140 is coupled to housing 105. Specifically, ejector 140a is releasably coupled to housing 105 via a press-fit arrangement between piston release 142c and housing release 105e. Cartridge 140b is coupled to housing 105 at housing distal end 105b via a press-fit arrangement.

FIG. 25 illustrates ingestible device 100b in a second state after device 100b has reached a desired location in the GI tract (e.g., the stomach, small intestine, large intestine) of a subject for delivering solid dosage form 150. In this example, device 100b is shown in the small intestine. As shown in FIG. 25, balloon 102 has been inflated by a gas generated within balloon interior 102e (as discussed above with reference to FIGS. 1-2) such that elongated section 102d is substantially aligned with a surface of the GI lumen wall and housing distal end 105b of housing 105 is oriented and positioned relative to (e.g., proximate to, or in contact with) the surface of the GI lumen wall. The generated gas within interior 102e applies a pressure (indicated by directional arrows 160) against an outer surface of piston 142 at piston proximal end 142a through first opening 105aa of housing 105. When the gas pressure reaches or exceeds a threshold value (e.g., a pressure associated with a fully or partially inflated state of balloon 102, such as a pressure of about 10-40 psi), piston release 142c overcomes the press-fit interface with housing release 105e. As a result, piston 142 moves axially relative to housing 105 along longitudinal axis 105d toward the GI lumen wall such that piston distal end 142b is advanced through first seal 148a to apply a force to a surface of solid dosage form 150 (e.g., a bottom surface of needle structure 152). In response to the applied force, solid dosage form 150 is advanced from container interior 146c as a projectile through second seal 148b along longitudinal axis 105d to penetrate through the GI lumen wall and into the peritoneum/peritoneal cavity.

In the example shown in FIG. 25, solid dosage form 150 is advanced partially through the GI lumen wall and into the peritoneal cavity of the subject. In other examples, solid dosage form 150 is ejected to a different penetration depth, such as into a layer of the GI lumen wall or fully into the peritoneum/peritoneal cavity for releasing one or more therapeutic agents contained therein. Upon penetrating to a desired depth, needle structure 152 may (partially or fully) degrade in situ to expose composition 154 to bodily tissue and/or fluids such that composition 154 can degrade to release one or more therapeutic agents contained therein. In this manner, one or more therapeutic agents contained in composition 154 can be delivered to the subject to treat a disease or condition.

Like the other examples described herein, one or more components of ingestible device 100b (e.g., balloon 102, needle structure 152) can be structured to degrade within the GI lumen wall or surrounding tissue, or other area within the GI tract upon completing delivery of solid dosage form 150.

Referring now to FIG. 26, a partial cross-sectional view of another example of housing 105 is illustrated as a housing 805 including a sleeve 805g for receiving cartridge 140b therein. Housing 805 may otherwise be structured the same as housing 105 with the same release feature 105e. Accordingly, for the sake of efficiency, like reference numerals refer to like components between examples, but are increased by an order of seven (e.g., housing interior 805c is equivalent to housing interior 105c).

As shown in FIG. 26, sleeve 805g has a substantially cylindrical shape and extends longitudinally from housing distal end 805b. Sleeve 805g may, advantageously, help to axially align cartridge 140b within housing interior 805c. Sleeve 805g may also help to retain cartridge 140b relative to housing 805 via the press-fit arrangement.

Referring now to FIG. 27, an example method 1000 of delivering a dosage form into a GI lumen wall or surrounding tissue of a subject using ingestible device 100 is illustrated. In a first step, the subject ingests device 100 (e.g., by swallowing device 100) (Step 1001). As a result of ingestion, enclosure 200 and/or outer coating 202 are at least partially (or may be fully) degraded upon device 100 reaching a desired location in the GI tract for delivery of the dosage form (Step 1002). In response to degradation of enclosure 200 and/or outer coating 202, release 116 is activated to cause balloon 102 to expand within the GI lumen (Step 1003). Expansion of balloon 102 within the GI lumen causes delivery assembly 104 to be oriented and positioned relative to the GI lumen wall (Step 1004). When a gas pressure within balloon interior 102e reaches or exceeds a threshold value, the gas pressure applied to payload delivery module 106 causes payload delivery module 106 to deliver the dosage form into the GI lumen wall or surrounding tissue thereof (Step 1005), as described herein. In this manner, device 100 can deliver one or more therapeutic agents contained in the dosage form to the subject for systemic uptake.

### Therapeutic Agents

As noted above, the ingestible devices, payload delivery modules, and assemblies described herein can contain at least one therapeutic agent. The identity of the therapeutic agent is not particularly limited. In one or more embodiments, the at least one therapeutic agent includes one or more selected from a small molecule, a peptide, a polypeptide, a protein, an antibody, a hormone, or a nucleic acid. In one or more embodiments, the at least one therapeutic agent is one or more selected from an immunosuppressive drug, a chemotherapy drug, a central nervous system (CNS) drug, an antidiabetic drug, an enzyme replacement therapy (ERT) drug, an anti-infective drug, a monoclonal antibody, an anti-coagulant, a blood clotting factor, insulin, an incretin or a combination thereof, or an oligonucleotide. In one or more embodiments, the at least one therapeutic agent includes an anti-sense oligonucleotide (ASO). In one or more embodiments, the ASO is MALAT1 ASO. In one or more embodiments, the at least one therapeutic agent includes a C-type natriuretic peptide (CNP). In one or more embodiments, the at least one therapeutic agent includes a programmed death-ligand 1 (PD-L1) protein. In one or more embodiments, the at least one therapeutic agent includes a monoclonal antibody. In one or more embodiments, the monoclonal antibody includes a TNF-α inhibiting antibody. In one or more embodiments, the TNF-α inhibiting antibody includes adalimumab or an analogue thereof. In one or more embodiments, the monoclonal antibody includes an anti-protease proprotein convertase subtilisin/kexin type 9 (anti-PCSK9) antibody. In one or more embodiments, the monoclonal antibody includes an anti-interleukin antibody. In one or more embodiments, the anti-interleukin antibody targets interleukin-4 and interleukin-13. In one or more embodiments, the anti-interleukin antibody includes dupilumab or an analogue thereof. In one or more embodiments, the anti-interleukin antibody targets interleukin-2. In one or more embodiments, the anti-interleukin antibody targets at least one of interleukin-12 or interleukin-23. In one or more embodiments, the anti-interleukin antibody includes ustekinumab or an analogue thereof. In one or more embodiments, the at least one therapeutic agent includes a parathyroid hormone (PTH) or an analogue thereof. In one or more embodiments, the at least one therapeutic agent includes amylin or an analogue thereof. In one or more embodiments, the at least one therapeutic agent includes one or more incretins or mimetics thereof selected from GLP-1, GLP-2, GIP, PYY, or glucagon receptor agonists.

Metastasis Associated Lung Adenocarcinoma Transcript 1 (MALAT1) is a large, infrequently spliced non-coding ribonucleic acid (RNA). MALAT1 closely relates to various pathological processes, ranging from diabetes complications to cancers. For example, MALAT1 regulates the expression of metastasis associated genes. Indeed, metastatic tumors have a dependency on MALAT1 and cannot survive without it. Thus, elevated MALAT1 expression is correlated with poor overall survival in various types of cancer, suggesting that this gene may be a prognostic factor. Genetic loss or systemic knockdown of MALAT1 using a MALAT1 ASO has been shown to slow tumor growth accompanied by significant differentiation into cystic tumors and a reduction in metastasis. Accordingly, MALAT1 ASO may be a potential therapy for inhibiting progression of certain cancers, such as lung cancer, pancreatic cancer, and cervical cancer. MALAT1 ASO is typically administered via a subcutaneous injection but can be delivered in accordance with the present disclosure. For example, MALAT1 formulated as a liquid dosage form can be used in an embodiment of an ingestible device described herein (i.e., embodiments of ingestible device 100 structured to deliver a fluid dosage form). For example, a suitable amount of MALAT1 formulated as a liquid dosage form can be loaded into an embodiment of an ingestible device as described herein structured to deliver a fluid dosage form, such as MALAT1 formulated as a liquid dosage form at a concentration of, for example, 10 mg/ml, at a dose of, for example, 0.5 mg/kg of the subject being treated.

Adalimumab, sold under the brand name HUMIRA® and biosimilars thereof, is a fully human, high-affinity, recombinant anti-tumor necrosis factor (TNF) alpha monoclonal antibody used to treat rheumatoid arthritis, ankylosing spondylitis, psoriasis, psoriatic arthritis, Crohn's disease, ulcerative colitis, hidradenitis suppurativa, juvenile idiopathic arthritis, and uveitis. Adalimumab is a molecule comprising 1330 amino acids and has a molecular weight of approximately 148 kDa. Adalimumab inhibits TNF alpha's interaction with p55 (TNFR1) and p75 (TNFR2) cell surface TNF receptors, which in turn interferes with cytokine-driven inflammatory processes. Adalimumab is currently available as an injection but can be delivered in accordance with the present disclosure. For example, adalimumab formulated as a liquid dosage form can be used in an embodiment of an ingestible device described herein (i.e., embodiments of ingestible device 100 structured to deliver a fluid dosage form). For example, a suitable amount of adalimumab formulated as a liquid dosage form (e.g., HUMIRA®) can be loaded into an embodiment of an ingestible device as described herein structured to deliver a fluid dosage form, as illustrated in Example 2. For example, each device may include a therapeutically effective dose of adalimumab in a range of about 3-11 mg, such as about 3 mg, 5 mg, 7 mg, 9 mg, 11 mg, or any value therebetween, or may include an amount of adalimumab effective to deliver any such therapeutically effective dose.

Dupilumab, sold under the brand name DUPIXENT®, is a monoclonal antibody blocking human IL-4 and IL-13, used for treating allergic diseases such as eczema, asthma, and nasal polyps which result in chronic sinusitis. Dupilumab is also used for the treatment of eosinophilic esophagitis and prurigo nodularis. Dupilumab is currently available as an injection but can be delivered in accordance with the present disclosure. For example, dupilumab formulated as a liquid dosage form can be used in an embodiment of an ingestible device described herein (i.e., embodiments of ingestible device 100 structured to deliver a fluid dosage form). For example, a suitable dose of dupilumab formulated as a liquid dosage form (e.g., DUPIXENT®) can be loaded into an embodiment of an ingestible device as described herein structured to deliver a fluid dosage form, as illustrated in Example 3. For example, each device may contain a therapeutically effective dose of dupilumab in a range of about 16-30 mg, such as about 16 mg, 18 mg, 20 mg, 22 mg, 24 mg, 26 mg, 28 mg, 30 mg, or any value therebetween, or may include an amount of dupilumab effective to deliver any such therapeutically effective dose. In other examples, each device may contain a therapeutically effective dose of dupilumab in a range of about 20-25 mg, such as about 20 mg, 21 mg, 22 mg, 23 mg, 24 mg, 25 mg, or any value therebetween, or may include an amount of dupilumab effective to deliver any such therapeutically effective dose.

RTJH23 is a unimolecular triagonist from Jiangsu Hengrui Pharmaceuticals. RTJH23 has a physiological action on GLP-1, GIP, and glucagon receptors resulting in weight loss due to early satiety (and glycemic control in diabetics). RTJH23 is currently deliverable as an injection but can be delivered in accordance with the present disclosure. For example, RTJH23 or another incretin agonist formulated as a liquid dosage form can be used in an embodiment of an ingestible device described herein (i.e., embodiments of ingestible device 100 structured to deliver a fluid dosage form). For example, a suitable amount of incretin triagonist formulated as a liquid dosage form (e.g., RTJH23) can be loaded into an embodiment of an ingestible device as described herein structured to deliver a fluid dosage form. For example, RTJH23 can be administered at a dose of 0.12 mg/kg and/or at a dose volume of 0.05 ml/kg, as illustrated in Example 4.

Ustekinumab, sold under the brand name STELARA® and biosimilars thereof, is a recombinant human IgG1κ monoclonal antibody produced in a murine cell line (Sp2/0) that acts as a human interleukin-12 and human interleukin-23 antagonist. Ustekinumab is used for treating moderate to severe plaque psoriasis, psoriatic arthritis, moderate to severe Crohn's disease, and moderate to severe ulcerative colitis. Ustekinumab is administered by subcutaneous or intravenous injection but can be delivered in accordance with the present disclosure. For example, ustekinumab formulated as a liquid dosage form can be used in an embodiment of an ingestible device described herein (i.e., embodiments of ingestible device 100 structured to deliver a fluid dosage form). For example, a suitable dose of ustekinumab formulated as a liquid dosage form (e.g., Celltrion CT-P43 biosimilar of STELARA®) can be loaded into an embodiment of an ingestible device as described herein structured to deliver a fluid dosage form, as illustrated in Example 5. For example, ustekinumab can be administered at a dose of 18 mg, as illustrated in Example 5. In other examples, each device may contain a therapeutically effective dose of ustekinumab in a range of about 10-22 mg, such as about 10 mg, 12 mg, 14 mg, 16 mg, 18 mg, 20 mg, 22 mg, or any value therebetween, or may include an amount of ustekinumab effective to deliver any such therapeutically effective dose.

Non-limiting examples of other specific therapeutic agents, approved dosages, and associated indications that could be treated in accordance with the present disclosure are listed in Table 1 below.

TABLE 1

| Drug Name (Brand | | | |
|---|---|---|---|
| Name) | Manufacturer | Indications | Approved Dosage |
| insulin detemir | Novo Nordisk A/S | Diabetes | 10 units daily |
| (LEVEMIR ®) | | | |
| insulin glargine | Sanofi | Diabetes | 10 units daily |
| (LANTUS ®) | | | |
| NORDITROPIN ® | Novo Nordisk A/S | Short stature/GH | 2 mg daily |
| | | deficiency | |
| insulin lispro | | Diabetes | 1 unit/kg/day |
| (HUMALOG ®, | | | |
| ADMELOG ®, | | | |
| LISPRO-PFC ®) | | | |
| metreleptin | AstraZeneca | Lipodystrophy | 2.5-10 mg daily |
| (MYALEPT ®) | Pharmaceuticals LP | | |
| aldesleukin | Clinigen Group plc | Metastatic Renal Cell | 0.037 mg/kg every 8 |
| (PROLEUKIN ®) | | Carcinoma and | hours with a 15-min |
| | | Metastatic Melanoma | infusion for a |
| | | | maximum of 14 doses; |
| | | | 9 days of rest, the |
| | | | schedule is repeated |
| | | | for another 14 doses, |
| | | | for a maximum of 28 |
| | | | doses per course, as |
| | | | tolerated. |
| adalimumab | AbbVie Inc. | RA, JIA, PsA, AS, CD, UC, | 40 mg biweekly |
| (HUMIRA ®) | | Ps, HS, UV | |
| adalimumab-adaz | Sandoz Inc. | RA, JIA, PsA, AS, CD, UC, | 40 mg biweekly |
| (HYRIMOZ ®) | | Ps | |
| adalimumab-adbm | Boehringer Ingelheim | RA, JIA, PsA, AS, CD, UC, | 41 mg biweekly |
| (CYLTEZO ®) | | Ps | |
| adalimumab-atto | Amgen Inc. | RA, JIA, PsA, AS, CD, UC, | 40 mg biweekly |
| (AMJEVITA ®) | | Ps | |
| liraglutide | Novo Nordisk A/S | Obesity | 3 mg daily |
| (SAXENDA ®) | | | |
| teduglutide | Takeda | Short bowel syndrome | 0.05 mg/kg daily |
| (GATTEX ®) | | | |
| albiglutide | GlaxoSmithKline LLC | T2D | 30-50 mg weekly |
| (TANZEUM ®) | | | |
| erenumab-aooe | Amgen | Migraine prevention | 70 mg once or twice |
| (AIMOVIG ®) | | | monthly |
| agalsidase beta | Genzyme Corporation | Fabry Disease | 1 mg/kg body weight |
| (FABRAZYME ®) | | | biweekly at a rate no |
| | | | more than 0.25 |
| | | | mg/min (15 mg/hour). |
| mogamulizumab- | Kyowa Kirin, Inc. | Relapsed or refractory | 1 mg/kg infused over |
| kpkc (POTELIGEO ®) | | mycosis fungoides; | 60 minutes biweekly |
| | | Sézary syndrome | |
| idursulfase | Shire Human Genetic | Mucopolysaccharidosis II | 0.5 mg/kg infused |
| (ELAPRASE ®) | Therapies, Inc. | | weekly |
| alemtuzumab | Genzyme | Leukemia & recurring MS | 30 mg IV 3x/week for |
| (CAMPATH ®, | | | 12 weeks (Leukemia) |
| LEMTRADA ®) | | | 12 mg IV 3x/week |
| | | | (MS) |
| alirocumab | Regeneron/Sanofi | Hyperlipidemia | 75 mg biweekly or 300 |
| (PRALUENT ®) | | | mg monthly |
| iaronidase | BioMarin | Mucopolysaccharidosis I | 0.58 mg/kg infused |
| (ALDURAZYME ®) | Pharmaceutical Inc. | | over 3-4 hours weekly |
| brentuximab | Seattle Genetics, Inc. | Hodgkin Lymphoma; | 1.2 mg/kg IV biweekly |
| vedotin (ADCETRIS ®) | | Anaplastic Large Cell | for 12 doses, disease |
| | | Lymphoma | progression, or |
| | | | unacceptable toxicity |
| pegfilgrastim | Amgen Inc. | Prevention of | 6 mg once per |
| (NEULASTA ®, | | Chemotherapy-induced | chemotherapy cycle in |
| FULPHILA ®, | | Neutropenia; | adults |
| UDENYCA ®, | | Hematopoietic | |
| ZIEXTENZO ®) | | Subsyndrome of Acute | |
| | | Radiation Syndrome | |
| | | (Neulasta only) | |
| pegfilgrastim-jmdb | Mylan GmbH | Prevention of | 6 mg once per |
| (FULPHILA ®) | | Chemotherapy-induced | chemotherapy cycle in |
| biosimilar to | | Neutropenia | adults |
| NEULASTA ® | | | |
| pembrolizumab | Merck Sharp & Dohme | Several cancers, | 2 mg/kg infused over |
| (KEYTRUDA ®) | Corp., a subsidiary of | including: Melanoma; | 30 minutes every 3 |
| | MERCK & CO., INC. | lung cancer; Lymphoma; | weeks until disease |
| | | Urothelial Carcinoma; | progression or |
| | | Gastric Cancer; Cervical | unacceptable toxicity |
| | | Cancer; Hepatocellular | |
| | | Carcinoma; Renal Cell | |
| | | Carcinoma | |
| etanercept | Immunex Corporation | AS, RA, PsA, Ps | 50 mg weekly |
| (ENBREL ®) | (Amgen) | | |
| etanercept-szzs | Sandoz Inc. | AS, RA, PsA | 50 mg weekly |
| asfotase alfa | Alexion | Hypophosphatasia (HPP) - | 2 mg/kg three times |
| (STRENSIQ ®) | Pharmaceuticals, Inc. | perinatal/infantile and | per week, or 1 mg/kg |
| | | juvenile onset | six times per week |
| sebelipase alfa | Alexion | Lysosomal Acid Lipase | 1-3 mg/kg intravenous |
| (KANUMA ®) | Pharmaceuticals Inc. | (LAL) Deficiency | infusion weekly |
| belantamab | GlaxoSmithKline | Multiple myeloma | 2.5 mg/kg infused |
| mafodotin-blmf | Intellectual Property | | over 30 mins every 3 |
| (BLENREP ®) | Development Ltd | | weeks |
| tocilizumab | Genentech, Inc | RA; Giant Cell Arteritis; | 4 mg/kg IV monthly or |
| (ACTEMRA ®) | | Cytokine Release | 162 mg SC biweekly |
| | | Syndrome | |
| galsulfase | Biomarin | Mucopolysaccharidosis | 1 mg/kg infused |
| (NAGLAZYME ®) | Pharmaceutical, Inc. | VI | weekly |
| velmanase alfa | Chiesi Farmaceutici | Alpha-mannosidosis | 1 mg/kg infused over |
| (LAMZEDE ®) EU | | | 50 mins weekly |
| ado-trastuzumab | Genentech, Inc. | Early or metastatic breast | 60-120 mg weekly |
| emtansine | | cancer | |
| (KADCYLA ®) | | | |
| rasburicase | Sanofi-Synthelabo Inc. | Hyperuricemia Caused by | 0.2 mg/kg infused |
| (ELITEK ®) | | Tumor Lysis | over 30 minutes daily |
| | | | for up to 5 days |
| sarilumab | Sanofi Aventis | RA | 200 mg biweekly |
| (KEVZARA ®) | | | |
| emicizumab-kxwh | Genentech | Hemophilia A | 3 mg/kg biweekly |
| (HEMLIBRA ®) | | | |
| brodalumab (SILIQ ®) | Manufactured for: | Psoriasis | 210 mg biweekly |
| | Valeant | | |
| | Pharmaceuticals North | | |
| | America LLC | | |
| | Manufactured by: | | |
| | Valeant | | |
| | Pharmaceuticals | | |
| | Luxembourg S.à.r.l. | | |
| nivolumab | Bristol-Myers Squibb | Melanoma; | 240 mg IV biweekly or |
| (OPDIVO ®) | Company | Hepatocellular | 480 mg IV monthly |
| | | Carcinoma; Advanced | until disease |
| | | Renal Cell Carcinoma; | progression or |
| | | Metastatic Colorectal | unacceptable toxicity |
| | | Cancer; SCLC; | |
| | | Lymphoma; NSCLC; | |
| | | Mesothelioma | |
| abatacept | Bristol-Myers Squibb | RA, JIA, PsA | 125-1000 mg weekly |
| (ORENCIA ®) | Company | | |
| pertuzumab | Genentech, Inc. | Early Breast Cancer; | 420 mg infused every |
| (PERJETA ®) | | Metastatic Breast Cancer | 3 weeks |
| vestronidase alfa- | Ultragenyx | Mucopolysaccharidosis | 4 mg/kg infused |
| vjbk (MEPSEVII ®) | Pharmaceutical Inc. | VII (MPS VII, Sly | biweekly over 4 hours |
| | | syndrome) | |
| ziv-aflibercept | sanofi-aventis, U.S., | Colorectal Cancer | 4 mg/kg infused |
| (ZALTRAP ®) | LLC | | biweekly over 1 hour |
| basiliximab | Novartis | Prophylaxis of acute | 20 mg before and |
| (SIMULECT ®) - | | organ rejection in renal | after surgery |
| ACUTE | | transplant | |
| trastuzumab | Genentech, Inc. | Breast Cancer; Gastric | 2 mg/kg infused |
| (HERCEPTIN ®) | | Cancer | weekly over 30 mins |
| elosulfase alfa | BioMarin | Morquio A Syndrome | 2 mg/kg infused over |
| (VIMIZIM ®) | Pharmaceutical Inc. | | 4 hours weekly |
| trastuzumab-anns | Amgen | HER2 overexpressing | 2 mg/kg infused |
| (KANJINTI ®) | | breast cancer | weekly over 30 mins |
| biosimilar to | | | |
| HERCEPTIN ® | | | |
| trastuzumab-dttb | Samsung Bioepis for | HER2-overexpressing | 2 mg/kg infused |
| and trastuzumab- | Merck | breast cancer | weekly over 30 mins |
| qyyp (ONTRUZANT ®, | | | |
| TRAZIMERA ®) | | | |
| trastuzumab-PKrb | CELLTRION, Inc. | HER2-overexpressing | 2 mg/kg infused |
| (HERZUMA ®) | | breast cancer | weekly over 30 mins |
| lanadelumab-flyo | Dyax Corp (Shire plc) | Prophylaxis to prevent | 300 mg biweekly |
| (TAKHZYRO ®) | | attacks of hereditary | |
| | | angioedema (HAE) | |
| dupilumab | Regeneron | Atopic Dermatitis; | 200-300 mg biweekly |
| (DUPIXENT ®) | Pharmaceuticals | Asthma | |
| Rilonacept | Regeneron | Cryopyrin Associated | 160 mg weekly |
| (ARCALYST ®) | Pharmaceuticals, Inc. | Periodic Syndromes | |
| | | (CAPS) | |
| KYNAMRO ® | Kastle Therapeutics | Homozygous familial | 200 mg weekly |
| | | hypercholesterolemia | |
| panitumumab | Amgen Inc. | Metastatic Colorectal | 6 mg/kg infused over |
| (VECTIBIX ®) | | Cancer | 60 minutes biweekly |
| darbepoetin alfa | Amgen Manufacturing | Chronic Kidney Disease- | 0.45 mcg/kg weekly or |
| (ARANESP ®) | Limited, a subsidiary of | Associated Anemia | monthly (depending |
| | Amgen Inc. | | on dialysis state) |
| mepolizumab | GlaxoSmithKline LLC | Severe Asthma; Churg- | 100-300 mg monthly |
| (NUCALA ®) | | Strauss Syndrome | |
| secukinumab | Novartis | Ps, PsA, AS | 150-300 mg monthly |
| (COSENTYX ®) | Pharmaceuticals | | |
| | Corporation | | |
| evolocumab | Amgen | Hyperlipidemia | 140 mg biweekly or |
| (REPATHA ®) | | | 420 mg monthly |
| omalizumab | Genentech, Inc. | Allergic Asthma; Chronic | 150-375 mg every 2-4 |
| (XOLAIR ®) | | Idiopathic Urticaria | weeks |
| belatacept | Bristol-Myers Squibb | Kidney transplant | 10 mg/kg six times; 5 |
| (NULOJIX ®) | Company | | mg/kg every 4 weeks |
| immunosuppressive | | | for maintenance |
| drug | | | |
| certolizumab pegol | UCB, Inc. | CD, RA, PsA, Ps | 400 mg monthly |
| (CIMZIA ®) | | | |
| belimumab | Human Genome | Lupus | 10 mg/kg monthly |
| (BENLYSTA ®) | Sciences, Inc. (a | | infused over 1 hour |
| | subsidiary of | | |
| | GlaxoSmithKline) | | |
| obinutuzumab | Genentech, Inc. | Follicular Lymphoma | 1000 mg IV bimonthly |
| (GAZYVA ®) | | | for up to 2 years |
| ipilimumab | Bristol-Myers Squibb | Melanoma; Renal Cell | 10 mg/kg every 3 |
| (YERVOY ®) | Company | Carcinoma; Metastatic | months for up to 3 |
| | | Colorectal Cancer; | years |
| | | NSCLC; Mesothelioma | |
| ustekinumab | Janssen Biotech, Inc. | Ps; PsA; CD; UC | 45-90 mg every 3 |
| (STELARA ®) | | | months |
| Denosumab | Amgen | postmenopausal women | 60 mg every 6 months |
| (PROLIA ®) | | with osteoporosis | |
| golimumab | Janssen Biotech, Inc. | RA, PsA, AS, UC | 50-100 mg monthly |
| (SIMPONI ®) | | | |
| burosumab-twza | Ultragenyx | X-linked | 1 mg/kg body weight |
| (CRYSVITA ®) | | hypophosphatemia | up to a maximum |
| | | | dose of 90 mg |
| | | | monthly |
| ixekizumab (TALTZ ®) | Eli Lilly and Company | Ps, PsA, AS | 80 mg monthly |
| tildrakizumab-asmn | Sun Pharmaceutical | Ps | 100 mg every 3 |
| (ILUMYA ®) | Industries | | months |
| guselkumab | Janssen Biotech | Ps | 100 mg bimonthly |
| (TREMFYA ®) | | | |
| Denosumab | Amgen | Multiple myeloma, giant | 120 mg monthly |
| (XGEVA ®) | | cell bone tumor | |
| galcanezumab-gnlm | Eli Lilly and Company | Migraine | 120 mg monthly |
| (EMGALITY ®) | | | |
| Satralizumab-mwge | Genentech | Neuromyelitis optica | 120 mg monthly |
| (ENSPRYNG ®) | | spectrum disorder | |
| | | (NMOSD) | |
| golimumab injection | Janssen Biotech, Inc. | RA, PsA, AS | 2 mg/kg bimonthly |
| (SIMPONI ARIA ®) | | | |
| canakinumab | Novartis | Tumor Necrosis Factor | 150 mg monthly |
| (ILARIS ®) | Pharmaceuticals | Receptor-Associated | |
| | Corporation | Periodic Syndrome; | |
| | | Hyperimmunoglobulin D | |
| | | Syndrome/Mevalonate | |
| | | Kinase Deficiency; | |
| | | Familial Mediterranean | |
| | | Fever; Still Disease; | |
| | | Cryopyrin-Associated | |
| | | Periodic Syndrome | |
| (GIVLAARI ®) | Alnylam | Acute hepatic porphyria | 175 mg monthly |
| reslizumab | Teva Respiratory, LLC | Asthma | 3 mg/kg infused |
| (CINQAIR ®) | | | monthly over 20-50 |
| | | | minutes |
| Romosuzumab | Amgen and UCB | Osteoporosis | 210 mg monthly |
| (EVENITY ®, | | | |
| ROMOSOZUMAB- | | | |
| AQQG ®) | | | |
| infliximab | Janssen Biotech, Inc. | RA, PsA, Ps, CD, UC, AS | 3-5 mg/kg bimonthly |
| (REMICADE ®) | | | |
| infliximab-dyyb | CELLTRION, Inc. | RA, PsA, Ps, CD, UC, AS | 3-5 mg/kg bimonthly |
| (INFLECTRA ®, | for | | |
| biosimilar to | Hospira, a Pfizer | | |
| REMICADE ®) | Company | | |
| infliximab-qbtx | Pfizer | CD, UC, RA, AS, PsA, Ps | 5 mg/kg bimonthly |
| (IXIFI ®) | | | |
| vedolizumab | Takeda | UC; CD | 300 mg IV bimonthly |
| (ENTYVIO ®) | | | |
| rituximab/rituximab- | Genentech, Inc. | Lymphoma; Leukemia; | 500 mg infusion every |
| pvvr (RITUXAN ®) | | RA | 6 months |
| rituximab-abbs | Teva Pharmaceuticals | Lymphoma; Leukemia; | 500 mg infusion every |
| (TRUXIMA ®) | | RA | 6 months |
| rituximab-arrx | Amgen | Lymphoma; Leukemia; | 500 mg infusion every |
| (RIABNI ®) | | RA | 6 months |
| rituximab-pvvr | Pfizer | Lymphoma; Leukemia; | 500 mg infusion every |
| (RUXIENCE ®) | | RA | 6 months |
| ocrelizumab | Genentech | MS | 600 mg intravenous |
| (OCREVUS ®) | | | infusion every 6 |
| | | | months |
| inebilizumab-cdon | Viela Bio Inc. | Neuromyelitis optica | 300 mg (90 min) |
| (UPLIZNA ®) | (AstraZeneca spinout) | spectrum disorder | infused semiannually |
| | | (NMOSD) | |
| ofatumumab | GLAXO GROUP | Chronic Lymphocytic | 1000 mg monthly up |
| (ARZERRA ®) | LIMITED | Leukemia | to 12 times |
| sacituzumab | Immunomedics, Inc. | Metastatic triple- | 10 mg/kg twice in 3 |
| govitecan-hzi | | negative breast cancer | weeks until disease |
| (TRODELVY ®) | | (mTNBC) | progression or |
| | | | unacceptable toxicity. |
| siltuximab | Manufactured by: | Castleman Disease | 11 mg/kg infused over |
| (SYLVANT ®) | Cilag AG | | 1 hour every 3 weeks |
| | Manufactured for: | | |
| | Janssen Biotech, Inc. | | |
| ramucirumab | Eli Lilly and Company | NSCLC; Gastric Cancer; | 8-10 mg/kg infused |
| (CYRAMZA ®) | | Colorectal Cancer; | biweekly |
| | | Hepatocellular | |
| | | Carcinoma | |
| (TEGSEDI ®) | Akcea Therapeutics | Transthyretin-mediated | 284 mg weekly |
| | | amyloidosis | |
| elotuzumab | Bristol-Myers Squibb | Multiple Myeloma | 10 mg/kg IV biweekly |
| (EMPLICITI ®) | Company | | or 20 mg/kg monthly |
| bevacizumab | Genentech, Inc | Colorectal Cancer; Non- | 10 mg/kg biweekly |
| (AVASTIN ®) | | Small Cell Lung Cancer; | |
| | | Renal Cell Carcinoma; | |
| | | Cervical Cancer; Ovarian, | |
| | | Fallopian Tube, or | |
| | | Peritoneal Cancer; | |
| | | Glioblastoma; | |
| | | Hepatocellular | |
| | | Carcinoma | |
| bevacizumab-awwb | Amgen | Metastatic Colorectal | 10 mg/kg biweekly |
| (MVASI ®) | | Cancer; Non-Small Cell | |
| | | Lung Cancer; Renal Cell | |
| | | Carcinoma; Glioblastoma | |
| bevacizumab-bvzr | Pfizer | Metastatic Colorectal | 10 mg/kg biweekly |
| (ZIRABEV ®) | | Cancer; Non-Small Cell | |
| | | Lung Cancer; Renal Cell | |
| | | Carcinoma; Cervical | |
| | | Cancer; Glioblastoma | |
| durvalumab | AstraZeneca | Metastatic urothelial | 10 mg/kg infused |
| (IMFINZI ®) | | carcinoma | biweekly over 60 min |
| atezolizumab | Genentech, Inc. | Urothelial Carcinoma; | 1200 mg infused over |
| (TECENTRIQ ®) | | Lung Cancer; Triple- | 30-60 minutes every 3 |
| | | Negative Breast Cancer; | weeks until disease |
| | | Hepatocellular | progression or |
| | | Carcinoma; Melanoma | unacceptable toxicity |
| avelumab | EMD Serono | Metastatic Merkel cell | 800 mg IV biweekly |
| (BAVENCIO ®) | | carcinoma | until disease |
| | | | progression or |
| | | | unacceptable toxicity |
| ibalizumab-uiyk | TaiMed Biologics | HIV | 800 mg biweekly |
| (TROGARZO ®) | | | |

### Embodiments

The following non-limiting embodiments are included in the present disclosure.

Embodiment 1: A payload delivery module for an ingestible device, the payload delivery module comprising: a piston including an interior; a hollow needle coupled to the piston; a membrane coupled to the piston to define a reservoir in the interior; a fluid dosage form disposed in the reservoir, the fluid dosage form comprising at least one therapeutic agent; and a valve coupled to the piston to control a flow of the fluid dosage form from the reservoir to the needle.

Embodiment 2: A payload delivery module for an ingestible device, the payload delivery module comprising: a piston including an interior; a hollow needle coupled to the piston; a plunger movably coupled to the piston to define a reservoir in the interior; a fluid dosage form disposed in the reservoir, the fluid dosage form comprising at least one therapeutic agent; and a valve coupled to the piston to control a flow of the fluid dosage form from the reservoir to the needle.

Embodiment 3: A payload delivery module for an ingestible device, the payload delivery module comprising: a piston including a base and an elongated section; a cartridge including a container; and a solid dosage form disposed in the container, the solid dosage form comprising at least one therapeutic agent.

Embodiment 4: A payload delivery module for an ingestible device, the payload delivery module comprising: a piston including an interior, a needle port, and a fill port; a membrane coupled to the piston to define a reservoir in the interior for containing a fluid dosage form; and a valve coupled to the piston between the membrane and the needle port.

Embodiment 5: A payload delivery module for an ingestible device, the payload delivery module comprising: a piston including an interior, a needle port, and a fill port; a plunger movably coupled to the piston to define a reservoir in the interior for containing a fluid dosage form; and a valve coupled to the piston between the plunger and the needle port.

Embodiment 6: The payload delivery module of any one of embodiments 1-5, wherein the piston is structured to be releasably coupled to, and movably disposed in, a housing.

Embodiment 7: The payload delivery module of any one of embodiments 1-2 and 4-5, wherein the valve is structured to control the flow of the fluid dosage form from the reservoir to the needle in response to axial movement of the module relative to a housing.

Embodiment 8: The payload delivery module of any one of embodiments 1-5, wherein the piston includes a lateral wall defining a release feature for releasably coupling the piston to a housing. Embodiment 9: The payload delivery module of embodiment 8, wherein the release feature is defined by an outer circumferential surface of the piston. Embodiment 10: The payload delivery module of embodiment 8 or 9, wherein the release feature comprises a press-fit feature.

Embodiment 11: The payload delivery module of any one of embodiments 1-2 and 4-5, wherein the valve comprises a seal coupled to the piston to contain the fluid dosage form in the reservoir. Embodiment 12: The payload delivery module of embodiment 11, wherein the valve further comprises a puncture member coupled to the piston adjacent to the seal, and wherein the puncture member is structured to move relative to the piston to pierce the seal. Embodiment 13: The payload delivery module of embodiment 12, wherein the puncture member is structured to create a substantially fluid-tight seal with the piston upon piercing the seal. Embodiment 14: The payload delivery module of embodiment 11, wherein the valve further comprises a base projecting from an outer surface of the piston and a tip coupled to the base. Embodiment 15: The payload delivery module of embodiment 14, wherein the base is integral with the piston. Embodiment 16: The payload delivery module of embodiment 14 or 15, wherein the base is structured to deform to cause the tip to move relative to the piston to pierce the seal.

Embodiment 17: The payload delivery module of any one of embodiments 1-2 and 4-5, wherein the valve comprises a base projecting from an outer surface of the piston and a plug releasably coupled to the piston adjacent to the base. Embodiment 18: The payload delivery module of embodiment 17, wherein the plug creates a substantially fluid tight seal with the piston between the membrane and the needle for containing the fluid dosage form in the reservoir. Embodiment 19: The payload delivery module of embodiment 17 or 18, wherein the base is structured to deform to cause the plug to release from the piston and allow the fluid dosage form to flow to the needle.

Embodiment 20: The payload delivery module of embodiment 1 or 4, wherein the membrane comprises a flexible material to allow for deformation of the membrane upon the valve opening by a pressure applied against an outer surface of the membrane.

Embodiment 21: The payload delivery module of embodiment 1 or 4, wherein the membrane comprises a metalized film or coating.

Embodiment 22: The payload delivery module of embodiment 2 or 5, wherein the plunger is structured to move relative to the piston upon the valve opening in response to a pressure applied against an outer surface of the plunger.

Embodiment 23: The payload delivery module of any one of embodiments 1-2 or 4-5, wherein the reservoir defines a volume for containing up to about 250 μl of fluid. Embodiment 24: The payload delivery module of embodiment 23, wherein the reservoir defines a volume for containing about 50-200 μl of fluid.

Embodiment 25: The payload delivery module of embodiment 1 or 4, wherein the needle has a critical length in a range of about 3.5-12 mm such that the needle delivers the fluid dosage form through the GI lumen wall into the peritoneal cavity.

Embodiment 26. The payload delivery module of embodiment 1 or 4, wherein the needle has a critical length in a range of about 1.5-3 mm such that the needle delivers the fluid dosage form into a layer of the GI lumen wall.

Embodiment 27: The payload delivery module of embodiment 1 or 4, wherein the needle comprises a biodegradable material to allow for degradation of at least a portion of the needle in a GI lumen wall or surrounding tissue thereof. Embodiment 28: The payload delivery module of embodiment 27, wherein the needle comprises a body and a tip coupled to the body, wherein the body is formed from a first material and the tip is formed from a second material, and wherein the second material has a hardness that is greater than a hardness of the first material.

Embodiment 29: The payload delivery module of any one of embodiments 1-5, further comprising a cover releasably coupled to the piston. Embodiment 30: The payload delivery module of embodiment 29, wherein the cover defines part of a housing for holding the payload delivery module.

Embodiment 31: The payload delivery module of embodiment 3, wherein the solid dosage form is shaped as or is contained in a needle structure.

Embodiment 32: The payload delivery module of embodiment 31, wherein the elongated section of the piston is structured to eject the needle structure from the container as a projectile into the GI lumen wall or surrounding tissue thereof.

Embodiment 33: The payload delivery module of any one of embodiments 1-3, wherein the at least one therapeutic agent is one or more selected from a small molecule, a peptide, a polypeptide, a protein, a hormone, an antibody, or a nucleic acid.

Embodiment 34: The payload delivery module of any one of embodiments 1-3, wherein the at least one therapeutic agent is one or more selected from an immunosuppressive drug, a chemotherapy drug, a central nervous system (CNS) drug, an antidiabetic drug, an enzyme replacement therapy (ERT) drug, an anti-infective drug, a C-type natriuretic peptide (CNP), a programmed death-ligand 1 (PD-L1) protein, a monoclonal antibody, an anti-coagulant, a blood clotting factor, insulin, an incretin or a combination thereof, or an oligonucleotide.

Embodiment 35: The payload delivery module of any one of embodiments 1-3, wherein the at least one therapeutic agent comprises an anti-sense oligonucleotide (ASO). Embodiment 36: The payload delivery module of embodiment 35, wherein the ASO is MALAT1.

Embodiment 37: The payload delivery module of any one of embodiments 1-3, wherein the at least one therapeutic agent comprises one or more blood clotting factors or mimetics thereof selected from Factor VIII, Factor IX, or Factor X.

Embodiment 38: The payload delivery module of any one of embodiments 1-3, wherein the at least one therapeutic agent comprises an anti-PCSK9 antibody.

Embodiment 39: The payload delivery module of any one of embodiments 1-3, wherein the at least one therapeutic agent comprises a TNF-α inhibiting antibody. Embodiment 40: The payload delivery module of embodiment 39, wherein the TNF-α inhibiting antibody comprises adalimumab or an analogue thereof.

Embodiment 41: The payload delivery module of any one of embodiments 1-3, wherein the at least one therapeutic agent comprises an anti-interleukin antibody. Embodiment 42: The payload delivery module of embodiment 41, wherein the anti-interleukin antibody targets interleukin 4 and interleukin 13. Embodiment 43: The payload delivery module of embodiment 42, wherein the anti-interleukin antibody comprises dupilumab or an analogue thereof. Embodiment 44: The payload delivery module of embodiment 41, wherein the anti-interleukin antibody targets at least one of interleukin 12 or interleukin 23. Embodiment 45: The payload delivery module of embodiment 44, wherein the anti-interleukin antibody comprises ustekinumab or an analogue thereof. Embodiment 46: The payload delivery module of embodiment 41, wherein the anti-interleukin antibody targets interleukin 2.

Embodiment 47: The payload delivery module of any one of embodiments 1-3, wherein the at least one therapeutic agent comprises parathyroid hormone (PTH) or an analogue thereof.

Embodiment 48: The payload delivery module of any one of embodiments 1-3, wherein the at least one therapeutic agent comprises amylin or an analogue thereof.

Embodiment 49: The payload delivery module of any one of embodiments 1-3, wherein the at least one therapeutic agent comprises one or more incretins or mimetics thereof selected from GLP-1, GLP-2, GIP, PYY, or glucagon receptor agonists.

Embodiment 50: A delivery assembly for an ingestible device, the delivery assembly comprising: a housing; and a payload delivery module of any one of embodiments 1-49 coupled to the housing. Embodiment 51: The delivery assembly of embodiment 50, wherein the housing comprises a proximal end, a distal end, a piston chamber located between the proximal and distal ends, and a housing release feature, wherein the proximal end includes a first opening for receiving a force in the interior and the distal end includes a second opening for discharging the dosage form. Embodiment 52: The delivery assembly of embodiment 51, wherein the housing further comprises a body and a cover coupled to the body, wherein the body includes the second opening and the piston chamber, and wherein the cover includes the first opening and the housing release feature. Embodiment 53: The delivery assembly of embodiment 51 or 52, wherein the piston is disposed in the piston chamber and is releasably coupled to the housing release feature. Embodiment 54: The delivery assembly of any one of embodiments 51-53, further comprising a seal coupled to the housing at the second opening. Embodiment 55: The delivery assembly of any one of embodiments 51-54, wherein the housing further comprises a vent channel for venting a gas. Embodiment 56: The delivery assembly of any one of embodiments 51-55, wherein the force is a gas pressure. Embodiment 57: The delivery assembly of embodiment 56, wherein the housing release feature is structured to release the piston in response to a threshold gas pressure applied to the module through the first opening, and wherein the threshold gas pressure is about 10-40 psi. Embodiment 58: The delivery assembly of any one of embodiments 51-57, wherein the housing release feature is located on a lateral wall of the housing. Embodiment 59: The delivery assembly of any one of embodiments 51-58, wherein the housing release feature comprises a press-fit feature. Embodiment 60: The delivery assembly of any one of embodiments 51-59, wherein the housing release feature extends circumferentially about the housing. Embodiment 61: The delivery assembly of any one of embodiments 51-60, wherein when the payload delivery module includes the fluid dosage form, the module is structured to move axially in the piston chamber to insert the hollow needle through the second opening into the GI lumen wall or surrounding tissue and to expel the fluid dosage form through the hollow needle into the GI lumen wall or surrounding tissue in response to the force received through the first opening. Embodiment 62: The delivery assembly of embodiment 61, wherein the fluid dosage form is expelled from the reservoir upon the valve opening in response to sufficient axial movement of the module relative to the housing. Embodiment 63: The delivery assembly of embodiment 61 or 62, wherein the fluid dosage form is expelled through the hollow needle after the hollow needle penetrates the GI lumen wall or surrounding tissue thereof. Embodiment 64: The delivery assembly of any one of embodiments 51-60, wherein when the payload delivery module includes the solid dosage form, the cartridge is coupled to the housing and the piston is structured to move axially in the piston chamber in response to the force received through the first opening to eject the solid dosage form from the container as a projectile into the GI lumen wall or surrounding tissue thereof.

Embodiment 65: An ingestible device comprising: an expandable member; and a delivery assembly of any one of embodiments 50-64 coupled to the expandable member. Embodiment 66: The ingestible device of embodiment 65, further comprising a gas generating mechanism coupled to the expandable member. Embodiment 67: The ingestible device of embodiment 66, wherein the gas generating mechanism is structured to generate a gas to cause the expandable member to expand at a location in a GI tract of a subject to orient and position the delivery assembly relative to a GI lumen wall. Embodiment 68: The ingestible device of embodiment 67, wherein the location is a small intestine of the subject. Embodiment 69: The ingestible device of any one of embodiments 66-68, wherein the gas generating mechanism comprises a plurality of reactants separated from each other by a degradable release. Embodiment 70: The ingestible device of any one of embodiments 65-69, wherein upon expansion of the expandable member, the delivery assembly is structured to deliver the dosage form through the GI lumen wall and into a peritoneum or a peritoneal cavity of the subject for systemic uptake of the at least one therapeutic agent. Embodiment 71: The ingestible device of any one of embodiments 65-69, wherein upon expansion of the expandable member, the delivery assembly is structured to deliver the dosage form into a layer of the GI lumen wall for systemic uptake of the at least one therapeutic agent. Embodiment 72: The ingestible device of any one of embodiments 65-71, wherein the expandable member comprises a balloon. Embodiment 73: The ingestible device of any one of embodiments 65-72, further comprising an ingestible enclosure, wherein the expandable member and the delivery assembly are disposed in the ingestible enclosure. Embodiment 74: The ingestible device of embodiment 73, wherein the ingestible enclosure comprises a biodegradable material to allow for degradation of at least a portion of the ingestible enclosure within the GI tract. Embodiment 75: The ingestible device of embodiment 73 or 74, further comprising a coating disposed over at least a portion of the ingestible enclosure, wherein the coating is structured to degrade at a selected pH in the GI tract. Embodiment 76: The ingestible device of any one of embodiments 73-75, wherein the ingestible enclosure is a size 00 or size 000 swallowable capsule.

Embodiment 77: A method of preparing an ingestible device for delivering a therapeutic agent into a GI lumen wall or surrounding tissue of a subject, the method comprising filling a dosage form comprising a therapeutic agent into a payload delivery module of any one of embodiments 1-49.

Embodiment 78: A method of delivering a therapeutic agent into a GI lumen wall or surrounding tissue of a subject in need thereof, the method comprising ingesting, by the subject, an ingestible device of any one of embodiments 65-76.

Embodiment 79: A method of delivering an oligonucleotide to a patient in need thereof, the method comprising: administering to the patient by swallowing an ingestible device of any one of embodiments 65-76, wherein the at least one therapeutic agent comprises a therapeutically effective dose of the oligonucleotide in liquid form; wherein upon swallowing the device, the expandable member expands in a GI tract of the patient to thereby deliver the therapeutically effective dose of the oligonucleotide into a lumen wall or surrounding tissue of the GI tract. Embodiment 80: The method of embodiment 79, wherein the oligonucleotide comprises an anti-sense oligonucleotide (ASO). Embodiment 81: The method of embodiment 80, wherein the ASO comprises MALAT1 ASO.

Embodiment 82: A method of delivering a TNF-α inhibiting antibody to a patient in need thereof, the method comprising: administering to the patient by swallowing an ingestible device of any one of claims 65-76, wherein the at least one therapeutic agent comprises a therapeutically effective dose of the TNF-α inhibiting antibody in liquid form; wherein upon swallowing the device, the expandable member expands in a GI tract of the patient to thereby deliver the therapeutically effective dose of the TNF-α inhibiting antibody into a lumen wall or surrounding tissue of the GI tract. Embodiment 83: The method of embodiment 82, wherein the TNF-α inhibiting antibody comprises adalimumab. Embodiment 84: The method of embodiment 83, wherein the adalimumab is HUMIRA® or a biosimilar thereof. Embodiment 85: The method of embodiment 84, wherein the therapeutically effective dose is about 3-11 mg.

Embodiment 86: A method of delivering an anti-interleukin antibody to a patient in need thereof, the method comprising: administering to the patient by swallowing an ingestible device of any one of embodiments 65-76, wherein the at least one therapeutic agent comprises a therapeutically effective dose of the anti-interleukin antibody in liquid form; wherein upon swallowing the device, the expandable member expands in the GI tract of the patient to thereby deliver the therapeutically effective dose of the anti-interleukin antibody into a lumen wall or surrounding tissue of the GI tract. Embodiment 87: The method of embodiment 86, wherein the anti-interleukin antibody targets interleukin 4 and interleukin 13. Embodiment 88: The method of embodiment 87, wherein the anti-interleukin antibody comprises dupilumab. Embodiment 89: The method of embodiment 88, wherein the dupilumab is DUPIXENT® or a biosimilar thereof. Embodiment 90: The method of embodiment 89, wherein the therapeutically effective dose is about 16-30 mg. Embodiment 91: The method of embodiment 89, wherein the therapeutically effective dose is about 20-25 mg. Embodiment 92: The method of embodiment 86, wherein the anti-interleukin antibody targets at least one of interleukin 12 or interleukin 23. Embodiment 93: The method of embodiment 92, wherein the anti-interleukin antibody comprises ustekinumab. Embodiment 94: The method of embodiment 93, wherein the ustekinumab is STELARA® or a biosimilar thereof. Embodiment 95: The method of any one of embodiments 86-94, wherein the therapeutically effective dose of the anti-interleukin antibody delivered to the patient produces a bioavailability in the patient that is substantially the same as a bioavailability of a subcutaneously administered dose of the anti-interleukin antibody. Embodiment 96: The method of any one of embodiments 86-94, wherein the therapeutically effective dose of the anti-interleukin antibody delivered to the patient produces a bioavailability in the patient that is higher than a bioavailability of a subcutaneously administered dose of the anti-interleukin antibody. Embodiment 97: The method of any one of embodiments 86-94, wherein the therapeutically effective dose of the anti-interleukin antibody delivered to the patient produces a Cₘₐₓ in the patient that is greater than a Cₘₐₓ of a subcutaneously administered dose of the anti-interleukin antibody. Embodiment 98: The method of any one of embodiments 86-94, wherein the therapeutically effective dose of the anti-interleukin antibody delivered to the patient produces a tₘₐₓ in the patient that is less than a tₘₐₓ of a subcutaneously administered dose of the anti-interleukin antibody.

Embodiment 99: A method of delivering at least one incretin to a patient in need thereof, the method comprising: administering to the patient by swallowing an ingestible device of any one of embodiments 65-76, wherein the at least one therapeutic agent comprises a therapeutically effective dose of the at least one incretin in liquid form; wherein upon swallowing the device, the expandable member expands in the GI tract of the patient to thereby deliver the therapeutically effective dose of the at least one incretin into a lumen wall or surrounding tissue of the GI tract. Embodiment 100: The method of embodiment 99, wherein the at least one incretin is an incretin triagonist comprising GLP-1, GIP, and glucagon receptor agonists. Embodiment 101: The method of embodiment 100, wherein the therapeutically effective dose of the incretin triagonist delivered to the patient produces a weight loss in the patient that is substantially the same as a weight loss produced by a subcutaneously administered dose of the incretin triagonist.

Embodiment 102: A modular delivery assembly for an ingestible device for delivering a dosage form into a GI lumen wall or surrounding tissue thereof of a subject, the delivery assembly comprising: a housing having a proximal end, a distal end, and an interior located between the proximal and distal ends; wherein the proximal end includes a first opening for receiving a force in the interior; wherein the distal end includes a second opening for discharging the dosage form; wherein the interior includes a receptacle structured to receive a module selected from a first payload delivery module comprising a fluid dosage form and a second payload delivery module comprising a solid dosage form; and wherein the housing further comprises a release feature to releasably couple the module to the housing. Embodiment 103: The modular delivery assembly of embodiment 102, wherein the first payload delivery module has a first delivery mode and the second payload delivery module has a second delivery mode different from the first delivery mode. Embodiment 104: The modular delivery assembly of embodiment 103, wherein the first delivery mode includes inserting a hollow needle into the GI lumen wall or surrounding tissue and discharging the fluid dosage form through the hollow needle into the GI lumen wall or surrounding tissue. Embodiment 105: The modular delivery assembly of embodiment 103 or 104, wherein the second delivery mode includes ejecting the solid dosage form from the second payload delivery module as a projectile into the GI lumen wall or surrounding tissue.

Embodiment 106: A delivery assembly for an ingestible device, the delivery assembly comprising: a housing including a proximal end, a distal end, a piston chamber located between the proximal and distal ends, and a release feature, wherein the proximal end includes a first opening for receiving a force in the interior and the distal end includes a second opening for discharging the dosage form; and a payload delivery module disposed in the housing, the payload delivery module comprising a piston and a dosage form including at least one therapeutic agent; wherein the piston is releasably coupled to the release feature and is structured to move axially in the piston chamber between the proximal and distal ends in response to the force to discharge the dosage form from the housing.

Embodiment 107: An ingestible device comprising: an expandable member; and a delivery assembly coupled to the expandable member, the delivery assembly including a fluid dosage form comprising at least one therapeutic agent; wherein the expandable member is structured to expand in a GI tract of a subject to position the delivery assembly relative to a GI lumen wall; and wherein upon expansion of the expandable member, the delivery assembly is structured to deliver the fluid dosage form through the GI lumen wall and into a peritoneum or a peritoneal cavity of the subject for systemic uptake of the at least one therapeutic agent.

Embodiment 108: An ingestible device comprising a housing; a piston movably disposed in the housing; a hollow needle coupled to the piston; a fluid dosage form disposed in the housing, the fluid dosage form comprising at least one therapeutic agent; and a force generating mechanism operatively coupled to the piston. Embodiment 109: The device of embodiment 108, wherein the hollow needle has a critical length in a range of about 3.5-12 mm such that upon ingestion of the device, the hollow needle is structured to deliver the fluid dosage form through a GI lumen wall into a peritoneal cavity of a subject for systemic uptake of the at least one therapeutic agent. Embodiment 110. The device of embodiment 108, wherein the hollow needle has a critical length in a range of about 1.5-3 mm such that upon ingestion of the device, the hollow needle is structured to deliver the fluid dosage form into a layer of the GI lumen wall for systemic uptake of the at least one therapeutic agent.

### EXAMPLES

The following examples are given to illustrate the present disclosure. It should be understood, however, that the disclosure is not to be limited to the specific conditions or details of these examples.

### Example 1—Oral Delivery of an Oligonucleotide Dosage Form to Canines

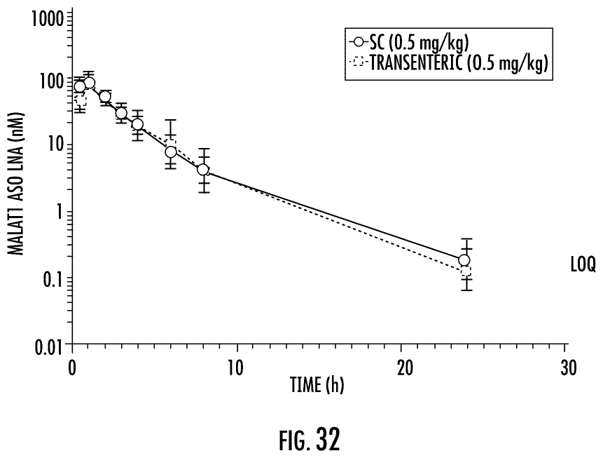

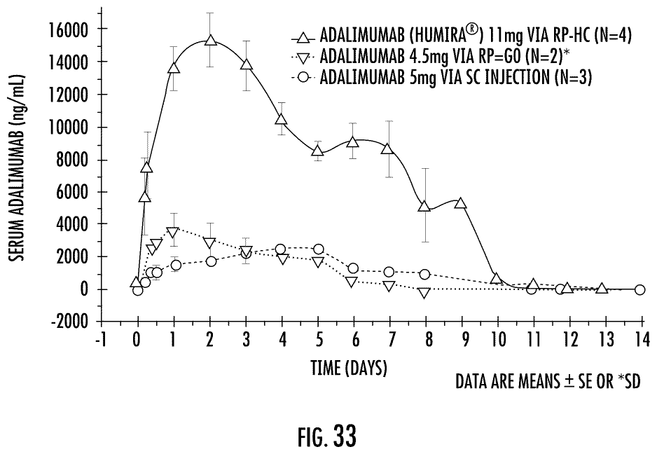

A preliminary pharmacokinetic (PK) study assessing oral delivery of a MALAT1 ASO liquid dosage form obtained from AstraZeneca® was conducted in dogs weighing about 8-12 kg. Four vials each having a volume of 4 ml and a MALAT1 concentration of 10 mg/ml were used for the study. Five anesthetized dogs each received a dose of 0.5 mg/kg of MALAT1 ASO as an intrajejunal injection from within the intestinal tract into the intestinal wall or through the intestinal wall into the peritoneum or peritoneal cavity (collectively referred to in the study as a “transenteric” route) via an endoscope to mimic oral administration of an ingestible device as described herein (i.e., embodiments of ingestible device 100 structured to deliver a fluid dosage form). After a washout period of one week, the same dogs each received a dose of 0.5 mg/kg of MALAT1 ASO as a subcutaneous (sc) injection to compare the two routes of administration. Serum blood samples were taken at regular intervals including at pre-dose, half-hour, 1 hour, 2 hours, 3 hours, 4 hours, 6 hours, 8 hours, and 24 hours after administration. Results are shown in FIG. 32.

As shown in FIG. 32, the transenteric and subcutaneous (sc) routes of administration produced similar PK profiles with comparable PK parameters including comparable max concentration (Cₘₐₓ), time to reach Cₘₐₓ (tₘₐₓ), half-life (t_{1/2}), and area under the curve (AUC). These results support the use of an ingestible device as described herein to orally deliver MALAT1 ASO and other oligonucleotide liquid dosage forms to humans for the treatment of, for example, certain cancers such as lung cancer, pancreatic cancer, and cervical cancer.

### Example 2—Oral Delivery of a Tumor Necrosis Factor (TNF) Alpha Inhibiting Antibody Dosage Form to Canines

A preliminary PK study assessing oral delivery of HUMIRA® (adalimumab) liquid dosage form using an embodiment of an ingestible device described herein (i.e., embodiments of ingestible device 100 structured to deliver a fluid dosage form) was conducted in dogs. After an overnight fast, four awake dogs weighing about 8-12 kg were each orally administered one device to swallow. Each device contained a dose of about 11 mg of HUMIRA® (adalimumab) in liquid form.

The gastrointestinal transit and deployment of devices were tracked fluoroscopically to determine the time of drug administration (t=0). Serum blood samples were collected using a validated and qualified ELISA over three weeks at intervals including 4 hours, 6 hours, 24 hours, and 36 hours post-dose, and daily from Day 2 (48 hours) to Day 21 (504 hours). All four devices deployed and ejected the dosage form. Further, all four dogs were able to excrete the devices after delivery with no significant findings on the remnants.

Referring to FIG. 33, the mean PK profile for the four orally administered devices was plotted against (1) the mean PK profile of an orally administered ingestible device containing a 4.5 mg solid dosage form of an adalimumab biosimilar that was previously conducted in two dogs using an ingestible device configured to deliver its payload into a GI lumen wall or surrounding tissue, and (2) the mean PK profile of a subcutaneously (sc) injected dose of 5 mg liquid dosage of an adalimumab biosimilar that was previously conducted in three dogs. As shown in FIG. 33, all four dogs that were administered the 11 mg liquid dosage using an embodiment of an ingestible device described herein demonstrated a signal for adalimumab and showed adalimumab levels below level of quantitation post day 12. Comparison of area under the curve (AUC) values for the 4.5 mg solid dosage, 5 mg SC injection, and the 11 mg liquid dosage demonstrated a dose-proportional increase in exposure levels for adalimumab. The coefficient of variation (% CV) between individual animals were similar between the different groups. These results support the use of an ingestible device as described herein to orally deliver liquid dosage forms of HUMIRA® and biosimilars thereof, or other TNF-α inhibiting antibodies, to humans for the treatment of, for example, certain autoimmune diseases such as rheumatoid arthritis, ankylosing spondylitis, psoriasis, psoriatic arthritis, Crohn disease, ulcerative colitis, hidradenitis suppurativa, juvenile idiopathic arthritis, and uveitis.

### Example 3—Oral Delivery of an Anti-Interleukin (targeting IL-4 and IL-13) Antibody Dosage Form to Canines

A preliminary PK study assessing oral delivery of dupilumab liquid dosage form using an embodiment of an ingestible device described herein (i.e., embodiments of ingestible device 100 structured to deliver a fluid dosage form) was conducted in dogs and compared to a subcutaneously (SC) administered control. After an overnight fast, six awake dogs weighing about 7.5-12.4 kg were each orally administered one device to swallow, while the SC control included three awake dogs that each received a dose of dupilumab via SC injection. Each ingestible device contained a dose of either 16.5 mg or 30 mg of dupilumab in a liquid form. The various dose groups are summarized below in Table 2.

TABLE 2

| Groups | Route | Dose (mg) | # Dogs |
|---|---|---|---|
| 1 & 2 | Oral | 16.5 | 6 |
| | | 30 | 8 |
| 3 & 4 | SC | 16.5 | 3 |
| | | 30 | 3 |

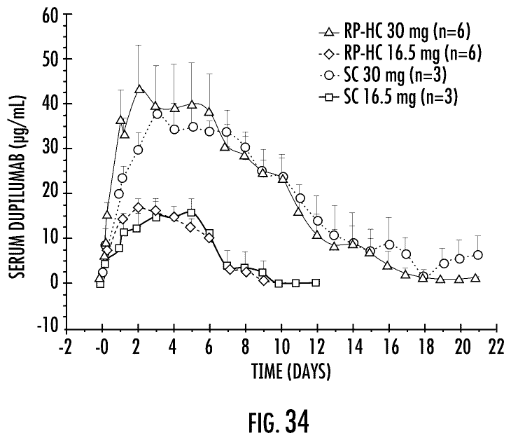

The gastrointestinal transit and deployment of the orally administered devices were tracked fluoroscopically to determine the time of drug administration (t=0). Serum blood samples were collected using a validated and qualified ELISA from ProteoGenix™ over three weeks at intervals including 4 hours, 6 hours, 24 hours, and 36 hours post-dose, and daily from Day 2 (48 hours) to Day 21 (504 hours). Twelve of the fourteen devices successfully delivered dupilumab, and both the oral and SC administered doses were well tolerated by all dogs with no clinically significant findings throughout the study. Mean results are shown in FIG. 34 and mean PK parameters are summarized below in Table 3.

TABLE 3

| | | | | Relative |
|---|---|---|---|---|
| | | | AUC₀₋ₜ | Bioavail- |
| | Cₘₐₓ | Tₘₐₓ | (μg · | ability |
| Group | (ug/mL) | (days) | day/mL) | (%) |
| 1 (Oral) 16.5 mg, N = 6 | 18 ± 2 | 2.2 ± 0.5 | 90 ± 12 | 89 |
| 2 (Oral) 30 mg, N = 6 | 47 ± 9 | 2.6 ± 1.0 | 405 ± 73 | 102 |
| 3 (SC) 16.5 mg, N = 3 | 16 ± 3 | 4.7 ± 0.3 | 101 ± 17 | — |
| 4 (SC) 30 mg, N = 3 | 40 ± 3 | 3.7 ± 0.7 | 398 ± 72 | — |

As shown in FIG. 34 and Table 3, oral administration of dupilumab via the ingestible devices produced a similar mean PK profile as the respective SC control, including a relative bioavailability that is substantially the same or higher. Oral administration of dupilumab via the ingestible devices also produced a higher Cₘₐₓ and shorter tₘₐₓ than the SC control. These results support the use of an ingestible device as described herein to orally deliver dupilumab and other anti-interleukin antibodies to humans for the treatment of, for example, certain allergic diseases such as asthma, eczema, chronic rhinosinusitis with nasal polyposis, and eosinophilic esophagitis.

### Example 4—Oral Delivery of an Incretin Triagonist Dosage Form to Canines

A preliminary pharmacokinetic (PK) and pharmacodynamic (PD) study assessing oral delivery of RTJH23 liquid dosage form was conducted in canines weighing 11-13 kilograms (kg). After an overnight fast, a first group of five canines each received a dose of 0.12 mg/kg of RTJH23 (with a dose volume of 0.05 ml/kg) as an intrajejunal injection from within the intestinal tract into the intestinal wall or through the intestinal wall into the peritoneum or peritoneal cavity (collectively referred to in the study as a “transenteric” route) via an endoscope to mimic oral administration of an ingestible device as described herein (i.e., embodiments of ingestible device 100 structured to deliver a fluid dosage form). A second group of five male canines each received a dose of 0.12 mg/kg of RTJH23 (with a dose volume of 0.04 ml/kg) as a subcutaneous injection.

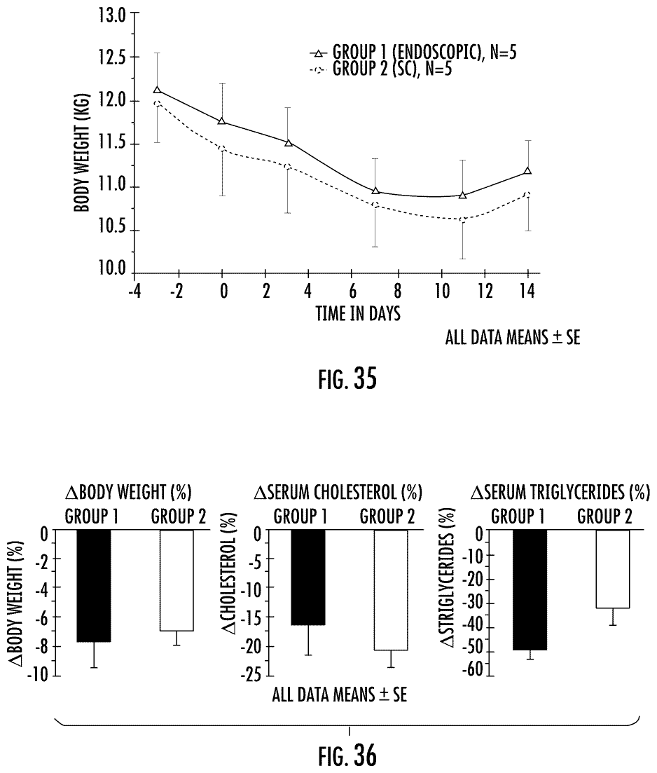

For the PK assessment, serum blood samples were collected using a validated and qualified ELISA over two weeks at intervals including at pre-dose, 15 minutes, 30 minutes, 1 hour, 2 hours, 4 hours, 6 hours, and 24 hours post-dose, and daily on Day 2, Day 4, Day 7, Day 10, and Day 14 post-dose. For the PD assessment, the canines were monitored daily to determine body weight, fasting blood glucose levels, liver enzymes (ALT and AST), lipid profiles (total cholesterol, triglycerides, LDL, HDL), and heart rate. Time course changes in body weight are summarized in FIG. 35, and peak decreases in body weights and serum lipids are summarized in FIG. 36.

As shown in FIGS. 35-36, the transenteric and SC routes of administration had substantially the same impact on body weight loss with greater than 10% weight loss observed by Day 7 in canines from both groups, which triggered IACUC-mandated supplemental high-fat feeding intervention to all canines for the remainder of the study. The rate of weight loss slowed after intervention on Day 7, and gradual weight re-gain was seen thereafter. Weight loss appeared to be due to early satiety leading to reduced caloric intake. Serum lipids also decreased in both groups along with the weight loss. In addition, fasted glucose levels tended to be mildly elevated for up to 72 hours post-dose in both groups. These results support the use of an ingestible device as described herein to orally deliver RTJH23 and other incretin dosage forms to humans for the treatment of, for example, a weight disorder, weight management, or other metabolic conditions.

### Example 5—Oral Delivery of an Anti-Interleukin (targeting IL-12 and/or IL-23) Antibody Dosage Form to Canines

A preliminary pharmacokinetic (PK) study assessing oral delivery of an ustekinumab biosimilar of STELARA® (CT-P43 from Celltrion, Inc.) in a liquid form using an embodiment of an ingestible device described herein (i.e., embodiments of ingestible device 100 structured to deliver a fluid dosage form) was conducted in canines and compared to a subcutaneously (SC) administered control. After an overnight fast, seven awake canines were each orally administered one device to swallow, while the SC control included three awake canines that each received a dose of 18 mg of CT-P43 via SC injection. Each ingestible device contained a dose of 18 mg of CT-P43 in a liquid form. One canine vomited a device after oral administration and was therefore excluded from the study.

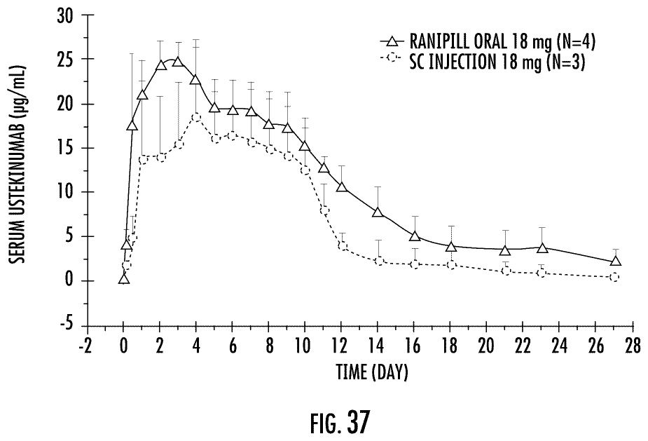

The gastrointestinal transit and deployment of the orally administered devices were tracked fluoroscopically to determine the time of drug administration (t=0). Serum blood samples were collected using a validated and qualified ELISA at intervals including 4 hours, 12 hours, and 24 hours post-dose, and daily from Day 2 to Day 12, and on Day 14, Day 16, Day 18, Day 21, Day 23, and Day 27. Four of the six devices successfully delivered ustekinumab, and both the oral and SC administered doses were well tolerated by all canines with no clinically significant findings throughout the study. Mean results are shown in FIG. 37 and mean PK parameters are summarized below in Table 4.

TABLE 4

| | | | | Relative |
|---|---|---|---|---|
| | Cₘₐₓ | Tₘₐₓ | AUC₀₋ₜ | Bioavailability |
| Group | (ug/mL) | (days) | (μg · day/mL) | (%) |
| RaniPill Oral | 29 ± 5 | 2 ± 1 | 294 ± 39 | 133 |
| 18 mg, N = 4 | | | | |
| SC Injection | 20 ± 8 | 5 ± 1 | 185 ± 56 | — |
| 18 mg, N = 3 | | | | |

As shown in FIG. 37 and Table 4, oral administration of ustekinumab via the ingestible devices (“RaniPill Oral”) produced a similar mean PK profile as the SC control (“SC Injection”), but with a relatively higher bioavailability, higher Cₘₐₓ, and shorter tₘₐₓ than the SC control. These results support the use of an ingestible device as described herein to orally deliver ustekinumab and other anti-interleukin antibodies to humans for the treatment of, for example, moderate to severe plaque psoriasis, psoriatic arthritis, moderate to severe Crohn's disease, and moderate to severe ulcerative colitis.

The foregoing description of various embodiments has been presented for purposes of illustration and description. It is not intended to limit the invention to the precise forms disclosed. Many modifications, variations and refinements will be apparent to practitioners skilled in the art. For example, embodiments of the device can be sized and otherwise adapted for various pediatric and neonatal applications as well as various veterinary applications. Also, those skilled in the art will recognize, or be able to ascertain using no more than routine experimentation, numerous equivalents to the specific devices and methods described herein. Such equivalents are considered to be within the scope of the present disclosure.

While the present disclosure has been described and illustrated with reference to specific embodiments thereof, these descriptions and illustrations do not limit the present disclosure. It can be clearly understood that various changes can be made, and equivalent components can be substituted within the embodiments, without departing from the true spirit and scope of the present disclosure. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further, for any positive recitation of a component, characteristic, constituent, feature, step or the like, embodiments of the invention specifically contemplate the exclusion of that component, value, characteristic, constituent, feature, step or the like. The illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. The specification and drawings are to be regarded as illustrative rather than restrictive. Modifications can be made to adapt a particular situation, material, composition of matter, method, or process to the objective, spirit and scope of the present disclosure. All such modifications are intended to be within the scope of the present disclosure. While the methods disclosed herein have been described with reference to particular operations performed in a particular order, it can be understood that these operations can be combined, sub-divided, or re-ordered to form an equivalent method without departing from the teachings of the present disclosure. Therefore, unless specifically indicated herein, the order and grouping of the operations are not limitations of the present disclosure.

1-74. (canceled)

75. A payload delivery module for an ingestible device, the payload delivery module comprising:
a piston including an interior;
a hollow needle coupled to the piston;
a membrane coupled to the piston to define a reservoir in the interior;
a fluid dosage form disposed in the reservoir, the fluid dosage form comprising at least one therapeutic agent; and
a valve coupled to the piston to control a flow of the fluid dosage form from the reservoir to the needle.

76. A payload delivery module for an ingestible device, the payload delivery module comprising:
a piston including an interior, a needle port, and a fill port;
a membrane coupled to the piston to define a reservoir in the interior for containing a fluid dosage form; and
a valve coupled to the piston between the membrane and the needle port.

77. The payload delivery module of claim 75, wherein the piston is structured to be releasably coupled to, and movably disposed in, a housing, optionally wherein the piston includes a lateral wall defining a release feature for releasably coupling the piston to a housing, further optionally wherein the release feature is defined by an outer circumferential surface of the piston, further optionally wherein the release feature comprises a press-fit feature.

78. The payload delivery module of claim 75, wherein the valve is structured to control the flow of the fluid dosage form from the reservoir to the needle in response to axial movement of the module relative to a housing, optionally wherein the valve comprises a seal coupled to the piston to contain the fluid dosage form in the reservoir, further optionally wherein the valve further comprises a base projecting from an outer surface of the piston and a tip coupled to the base, further optionally wherein the base is integral with the piston.

79. The payload delivery module of claim 78, wherein the valve further comprises a puncture member coupled to the piston adjacent to the seal, and wherein the puncture member is structured to move relative to the piston to pierce the seal, optionally wherein the puncture member is structured to create a substantially fluid-tight seal with the piston upon piercing the seal, further optionally wherein the base is structured to deform to cause the tip to move relative to the piston to pierce the seal.

80. The payload delivery module of claim 75, wherein the membrane comprises a flexible material to allow for deformation of the membrane upon the valve opening by a pressure applied against an outer surface of the membrane, optionally wherein the membrane comprises a metalized film or coating.

81. The payload delivery module of claim 75, wherein the reservoir defines a volume for containing up to about 250 μl of fluid, optionally wherein the reservoir defines a volume for containing about 50-200 μl of fluid.

82. The payload delivery module of claim 75, wherein (i) the needle has a critical length in a range of about 3.5-12 mm such that the needle delivers the fluid dosage form through the GI lumen wall into the peritoneal cavity, or (ii) wherein the needle has a critical length in a range of about 1.5-3 mm such that the needle delivers the fluid dosage form into a layer of the GI lumen wall.

83. The payload delivery module of claim 75, further comprising a cover releasably coupled to the piston, optionally wherein the cover defines part of a housing for holding the payload delivery module.

84. The payload delivery module of claim 75, wherein the at least one therapeutic agent is one or more selected from a small molecule, a peptide, a polypeptide, a protein, a hormone, an antibody, or a nucleic acid, optionally wherein the at least one therapeutic agent is one or more selected from an immunosuppressive drug, a chemotherapy drug, a central nervous system (CNS) drug, an antidiabetic drug, an enzyme replacement therapy (ERT) drug, an anti-infective drug, a C-type natriuretic peptide (CNP), a programmed death-ligand 1 (PD-L1) protein, a monoclonal antibody, an anti-coagulant, a blood clotting factor, insulin, an incretin or a combination thereof, or an oligonucleotide, further optionally wherein the at least one therapeutic agent comprises one or more therapeutic agents selected from an anti-sense oligonucleotide (ASO), optionally wherein the ASO is MALAT1; one or more blood clotting factors or mimetics thereof selected from Factor VIII, Factor IX, or Factor X; an anti-PCSK9 antibody; a TNF-α inhibiting antibody, optionally wherein the TNF-α inhibiting antibody comprises adalimumab or an analogue thereof; an anti-interleukin antibody, optionally wherein the anti-interleukin antibody targets interleukin 4 and interleukin 13 (e.g., dupilumab or an analogue thereof) or at least one of interleukin 12 or interleukin 23 (e.g., ustekinumab or an analogue thereof), or interleukin 2; parathyroid hormone (PTH) or an analogue thereof, amylin or an analogue thereof; and one or more incretins or mimetics thereof, optionally selected from GLP-1, GLP-2, GIP, PYY, or glucagon receptor agonists.

85. A delivery assembly for an ingestible device, the delivery assembly comprising:
a housing; and
a payload delivery module of claim 75 coupled to the housing.

86. The delivery assembly of claim 85, wherein the housing comprises a proximal end, a distal end, a piston chamber located between the proximal and distal ends, and a housing release feature, wherein the proximal end includes a first opening for receiving a force in the interior and the distal end includes a second opening for discharging the dosage form, optionally wherein the piston is disposed in the piston chamber and is releasably coupled to the housing release feature, optionally further comprising a seal coupled to the housing at the second opening.

87. The delivery assembly of claim 86, wherein the housing further comprises a body and a cover coupled to the body, wherein the body includes the second opening and the piston chamber, and wherein the cover includes the first opening and the housing release feature.

88. The delivery assembly of claim 86, wherein the housing further comprises a vent channel for venting a gas, wherein the force is a gas pressure, and wherein the housing release feature is structured to release the piston in response to a threshold gas pressure applied to the module through the first opening, and wherein the threshold gas pressure is about 10-40 psi.

89. The delivery assembly of claim 86, wherein the payload delivery module includes the fluid dosage form, and the module is structured to move axially in the piston chamber to insert the hollow needle through the second opening into the GI lumen wall or surrounding tissue and to expel the fluid dosage form through the hollow needle into the GI lumen wall or surrounding tissue in response to the force received through the first opening, optionally wherein the fluid dosage form is expelled from the reservoir upon the valve opening in response to sufficient axial movement of the module relative to the housing and/or wherein the fluid dosage form is expelled through the hollow needle after the hollow needle penetrates the GI lumen wall or surrounding tissue thereof.

90. An ingestible device comprising:
an expandable member; and
a delivery assembly of claim 85 coupled to the expandable member.

91. The ingestible device of claim 90, further comprising a gas generating mechanism coupled to the expandable member, wherein the gas generating mechanism is structured to generate a gas to cause the expandable member to expand at a location in a GI tract of a subject to orient and position the delivery assembly relative to a GI lumen wall, wherein upon expansion of the expandable member, the delivery assembly is structured to deliver the dosage form through the GI lumen wall and into a peritoneum or a peritoneal cavity of the subject for systemic uptake of the at least one therapeutic agent.

92. The ingestible device of claim 91, wherein the delivery assembly is structured to, upon expansion of the expandable member, deliver the dosage form into a layer of the GI lumen wall for systemic uptake of the at least one therapeutic agent.

93. An ingestible device comprising:
a housing;
a piston movably disposed in the housing;
a hollow needle coupled to the piston;
a fluid dosage form disposed in the housing, the fluid dosage form comprising at least one therapeutic agent; and
a force generating mechanism operatively coupled to the piston.

94. The device of claim 93, wherein (i) the hollow needle has a critical length in a range of about 3.5-12 mm such that upon ingestion of the device, the hollow needle is structured to deliver the fluid dosage form through a GI lumen wall into a peritoneal cavity of a subject for systemic uptake of the at least one therapeutic agent, or (ii) the hollow needle has a critical length in a range of about 1.5-3 mm such that upon ingestion of the device, the hollow needle is structured to deliver the fluid dosage form into a layer of the GI lumen wall for systemic uptake of the at least one therapeutic agent.
